(19) United States
(12) Patent Application Publication
(10) Pub. No.: US 2023/0357795 A1
(43) Pub. Date: Nov. 9, 2023

(54) AAV-MEDIATED HOMOLOGY-INDEPENDENT TARGETED INTEGRATION GENE EDITING FOR CORRECTION OF DIVERSE DMD MUTATIONS IN PATIENTS WITH MUSCULAR DYSTROPHY

(71) Applicant: RESEARCH INSTITUTE AT NATIONWIDE CHILDREN'S HOSPITAL, Columbus, OH (US)

(72) Inventors: Kevin Flanigan, Columbus, OH (US); Anthony Aaron Stephenson, Columbus, OH (US); Julian Havens, West Chester, OH (US)

(21) Appl. No.: 18/245,025

(22) PCT Filed: Sep. 15, 2021
(86) PCT No.: PCT/US21/50461

§ 371 Date: Mar. 13, 2023

Related U.S. Application Data
(60) Provisional application No. 63/078,428, filed on Sep. 15, 2020.
(60) Provisional application No. 63/180,232, filed on Apr. 27, 2021.

(51) Int. Cl. C12N 15/86; C12N 15/90; C12N 9/22; A61P 43/00; C12N 15/10

(52) U.S. Cl. CPC C12N 15/86; C12N 15/907; C12N 9/22; A61P 43/00; C12N 15/102; C12N 2750/14143; C12N 2310/20

(57) ABSTRACT

Disclosed herein are products, methods, and uses for a new gene therapy for treating, ameliorating, delaying the progression of, and/or preventing a muscular dystrophy involving a mutation amenable to DNA repair including, but not limited to, any mutation involving, surrounding, or affecting various regions of the DMD gene. Specifically, the disclosure provides products and methods for fixing diverse DMD mutations by replacement of large segments of the DMD gene comprising multiple exons, using CRISPR/Cas9 and Homology-Independent Targeted-Integration (HITI) to accomplish high efficiency knock-in or make large replacements using the non-homologous end-joining (NHEJ) DNA repair pathway, previously not achievable. In particular, the disclosure provides products, methods and uses for the replacement of DMD exons 1-19, 2-19, or 41-55.

### INCORPORATION BY REFERENCE OF THE SEQUENCE LISTING

This application contains, as a separate part of disclosure, a Sequence Listing in computer-readable form (filename: 55650PC_Seqlisting.txt; Size: 105,386 bytes: Created: Sep. 14, 2021) which is incorporated by reference herein in its entirety.

### FIELD

This disclosure relates to the field of gene therapy for the treatment of muscular dystrophy. More particularly, the disclosure provides products, methods, and uses for a new gene therapy for treating, ameliorating, delaying the progression of, and/or preventing a muscular dystrophy involving a mutation amenable to DNA repair including, but not limited to, any mutation involving, surrounding, or affecting various regions of the DMD gene crossing multiple exons. Specifically, the disclosure provides products and methods for fixing diverse DMD mutations by replacement of large segments of the DMD gene, previously not achievable. The disclosure provides products and methods for addressing mutations within the DMD locus in a region encompassed by introns 1-19 and introns 40-55. In some aspects, the mutation is involving, surrounding, or affecting DMD exons 1-19, 2-19, or 41-55. In some aspects, the mutation is encompassed by the DMD promoter, the 5′ untranslated region, as well as exon 1 through intron 19. In some aspects, the disclosure provides products and methods for the replacement of DMD exons 1-19, 2-19, or 41-55. However, the disclosure provides a method which is applicable to the replacement of other regions of the DMD gene as well.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). It is the most common severe childhood form of muscular dystrophy affecting 1 in 5000 newborn males. DMD is caused by mutations in the DMD gene leading to absence of dystrophin protein (427 KDa) in skeletal and cardiac muscles, as well as the gastrointestinal tract and retina. Dystrophin not only protects the sarcolemma from eccentric contractions, but also anchors a number of signaling proteins in close proximity to sarcolemma. Another form of MD is Becker Muscular Dystrophy (BMD). BMD, like DMD, is a genetic disorder that gradually makes the body's muscles weaker and smaller. BMD affects the muscles of the hips, pelvis, thighs, and shoulders, as well as the heart, but is known to cause less severe problems than DMD.

Many clinical cases of DMD are linked to deletion mutations in the DMD gene. In contrast to the deletion mutations, DMD exon duplications account for around 5% of disease-causing mutations in unbiased samples of dystrophinopathy patients [Dent et al., Am J Med Genet, 134(3): 295-298 (2005)], although in some catalogues of mutations the number of duplications is higher, including that published by the United Dystrophinopathy Project by Flanigan et al. [Hum Mutat, 30(12): 1657-1666 (2009)], in which it was 11%. BMD is also caused by a change in the dystrophin gene, which makes the protein too short. The flawed dystrophin puts muscle cells at risk for damage with normal use. See also, U.S. Patent Application Publication Nos. 2012/0077860, published Mar. 29, 2012; 2013/0072541, published Mar. 21, 2013; and 2013/0045538, published Feb. 21, 2013.

A deletion of exon 45 is one of the most common deletions found in DMD patients, whereas a deletion of exons 44 and 45 is generally associated with BMD [Anthony et al., JAMA Neurol 71:32-40 (2014)]. Thus, if exon 44 could be bypassed in pre-messenger RNA (mRNA), transcripts of these DMD patients, this would restore the reading frame and enable the production of a partially functional BMD-like dystrophin [Aartsma-Rus et al., Nucleic Acid Ther 27(5): 251-259 (2017)]. In fact, it appears that many patients with a deletion bordering on exon 45, skip exon 44 spontaneously, although at very low levels. This results in slightly increased levels of dystrophin when compared with DMD patients carrying other deletions, and most likely underlies the less severe disease progression observed in these patients compared with DMD patients with other deletions [Anthony et al., supra; Pane et al., PLoS One 9:e83400 (2014); van den Bergen et al., J Neuromuscul Dis 1:91-94 (2014)].

Despite many lines of research following the identification of the DMD gene, treatment options are limited. Thus, there remains a need in the art for treatments for MDs, including DMD. The most advanced therapies include those that aim at restoration of the missing protein, dystrophin, using mutation-specific genetic approaches.

### SUMMARY

The disclosure provides products, methods, and uses for a new gene therapy for treating, ameliorating, delaying the progression of, and/or preventing a muscular dystrophy involving a mutation amenable to DNA repair including, but not limited to, any mutation involving, surrounding, or affecting various regions of the DMD gene. Specifically, the disclosure provides products and methods for fixing diverse DMD mutations by replacement of large segments of the DMD gene, previously not achievable. The disclosure provides products and methods for addressing mutations within the DMD locus in a region encompassed by introns 1-19 and introns 40-55. In some aspects, the mutation is involving, surrounding, or affecting DMD exons 1-19, 2-19, or 41-55. In some aspects, the mutation is encompassed by the DMD promoter, the 5′ untranslated region, as well as exon 1 through intron 19. In some aspects, the disclosure provides products and methods for the replacement of DMD exons 41-55, exons 1-19, or exons 2-19. In some aspects, the disclosure provides products and methods for knock-in of a synthetic promoter and a natural or modified coding sequence for DMD exons 1-19. However, the disclosure provides a method which is applicable to other regions of the DMD gene as well.

More particularly, the disclosure provides nucleic acids encoding guide RNAs (gRNAs), nucleic acids comprising coding sequences lacking internal introns flanked by native or synthetic introns comprising splice sites required for transcript maturation, and recombinant adeno-associated virus (rAAV) comprising the nucleic acids. The products and methods provided herein provide an altered form of dystrophin protein for use in treating a muscular dystrophy resulting from a mutation involving, surrounding, or affecting various regions of the DMD gene. In some aspects, the mutation is involving, surrounding, or affecting mutations within the DMD locus in a region encompassed by introns 1-19 and introns 40-55. In some aspects, the mutation is involving, surrounding, or affecting DMD exons 1-19, 2-19, or 41-55. In some aspects, the mutation is encompassed by the DMD promoter, the 5′ untranslated region, as well as exon 1 through intron 19.

The “homology-independent targeted integration” (HITI) technology described herein as being used herein the methods of the disclosure includes three components: i) Cas9 to generate DNA double-stranded breaks at user-chosen sites, ii) guide RNAs (gRNAs) to guide Cas9 to user-chosen DNA sites on the DMD gene, and iii) a donor DNA containing the desired knock-in DMD sequence flanked by one or more of the gRNA target sites. Importantly, HITI uses the non-homologous end-joining (NHEJ) DNA repair pathway in cells to catalyze knock-in of linear DNA sequences into the genome at Cas9 cut sites.

The disclosure provides a nucleic acid encoding a Duchenne muscular dystrophy (DMD) gene-targeting guide RNA (gRNA) comprising the nucleotide sequence set forth in any one of SEQ ID NOs: 1-37 or a variant thereof comprising at least or about 80% identity to the nucleotide sequence set forth in any one of SEQ ID NOs: 1-37; or a nucleotide sequence that specifically hybridizes to a target nucleic acid encoding DMD comprising the nucleotide sequence set forth in any one of SEQ ID NOs: 112-148.

The disclosure provides a nucleic acid comprising a donor DNA sequence encoding knock-in donor sequence of the DMD gene comprising the nucleotide sequence set forth in SEQ ID NO: 149, 152, 155, 158, 172, 176, 187, or 188 or a variant thereof comprising at least or about 80% identity to the nucleotide sequence set forth in SEQ ID NO: 149, 152, 155, 158, 172, 176, 187, or 188.

In some aspects, these nucleic acids further comprise a promoter sequence. In some aspects, the promoter is any of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

The disclosure provides a composition comprising these nucleic acids. In some aspects, the disclosure provides a vector comprising these nucleic acids. In some aspects, the vector is an adeno-associated virus. In some aspects, the adeno-associated virus lacks rep and cap genes. In some aspects, the adeno-associated virus is a recombinant AAV (rAAV) or a self-complementary AAV (scAAV). In some aspects, the AAV is AAV1, AAV2, AAV3, AAV4, AAV5, AAV6, AAV7, AAV8, AAV9, AAV10, AV11, AAV12, AV13, AAVanc80, or AAV rh.74. In some more particular aspects, the AAV is rAAV9. The disclosure provides a composition comprising such an AAV and a pharmaceutically acceptable carrier.

The disclosure provides a method for replacing one or more missing, duplicated, aberrant, or aberrantly-spliced exons or missing or aberrant introns in the DMD gene in a cell, the method comprising transfecting the cell with a nucleic acid encoding a first DMD-targeting guide RNA (gRNA) targeting intron 1 and a nucleic acid encoding a second DMD-targeting gRNA targeting intron 19; or a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19; a nucleic acid comprising a donor DNA sequence encoding knock-in donor sequence of exons 2-19 the DMD gene flanked on each side of the donor sequences by a genomic Cas9 cut site; and a nucleic acid encoding a Cas9 enzyme or a functional fragment thereof. The disclosure also provides a method for replacing one or more missing, duplicated, aberrant, or aberrantly-spliced exons or missing or aberrant introns in the DMD gene in a cell, the method comprising transfecting the cell with a vector comprising a nucleic acid encoding a first DMD-targeting guide RNA (gRNA) targeting intron 1 and a nucleic acid encoding a second DMD-targeting gRNA targeting intron 19; or a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19; a nucleic acid comprising a donor DNA sequence encoding knock-in donor sequence of exons 2-19 the DMD gene flanked on each side of the donor sequences by a genomic Cas9 cut site; and a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28. In some aspects, the nucleic acid encoding a first DMD-targeting gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139. In some aspects, the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37. In some aspects, the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148. In some aspects, the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158.

The disclosure provides a method for replacing one or more missing, duplicated, aberrant, or aberrantly-spliced exons or missing or aberrant introns in the DMD gene in a cell, the method comprising transfecting the cell with a nucleic acid encoding a first DMD-targeting guide RNA (gRNA) targeting intron 40 and a nucleic acid encoding a second DMD-targeting gRNA targeting intron 55; or a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55; a nucleic acid comprising a donor DNA sequence encoding knock-in donor sequence of exons 41-55 of the DMD gene flanked on each side of the donor sequences by a genomic Cas9 cut site; and a nucleic acid encoding a Cas9 enzyme or a functional fragment thereof. The disclosure also provides a method for replacing one or more missing, duplicated, aberrant, or aberrantly-spliced exons or missing or aberrant introns in the DMD gene in a cell, the method comprising transfecting the cell with a vector comprising a nucleic acid encoding a first DMD-targeting guide RNA (gRNA) targeting intron 40 and a nucleic acid encoding a second DMD-targeting gRNA targeting intron 55; or a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55; a nucleic acid comprising a donor DNA sequence encoding knock-in donor sequence of exons 41-55 of the DMD gene flanked on each side of the donor sequences by a genomic Cas9 cut site; and a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6. In some aspects, the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117. In some aspects, the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9. In some aspects, the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120. In some aspects, the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188. In some aspects, expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the cell is a human cell. In some aspects, the human cell is in a human subject. In some aspects, the human subject has a muscular dystrophy or suffers from a muscular dystrophy. In some aspects, the muscular dystrophy is Duchene Muscular Dystrophy (DMD) or Becker Muscular Dystrophy (BMD).

The disclosure provides a method of treating a subject suffering from one or more missing, duplicated, aberrant, or aberrantly-spliced exons or missing or aberrant introns in the DMD gene in a cell, the method comprising administering to the subject an effective amount of a nucleic acid encoding a first DMD-targeting guide RNA (gRNA) targeting intron 1 and a nucleic acid encoding a second DMD-targeting gRNA targeting intron 19; or a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19; a nucleic acid comprising a donor DNA sequence encoding knock-in donor sequence of exons 2-19 the DMD gene flanked on each side of the donor sequences by a genomic Cas9 cut site; and a nucleic acid encoding a Cas9 enzyme or a functional fragment thereof. The disclosure also provides a method of treating a subject suffering from one or more missing, duplicated, aberrant, or aberrantly-spliced exons or missing or aberrant introns in the DMD gene in a cell, the method comprising administering to the subject an effective amount of a vector comprising a nucleic acid encoding a first DMD-targeting guide RNA (gRNA) targeting intron 1 and a nucleic acid encoding a second DMD-targeting gRNA targeting intron 19; or a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19; a nucleic acid comprising a donor DNA sequence encoding knock-in donor sequence of exons 2-19 the DMD gene flanked on each side of the donor sequences by a genomic Cas9 cut site; and a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28. In some aspects, the nucleic acid encoding a first DMD-targeting gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139. In some aspects, the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37. In some aspects, the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148. In some aspects, the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158.

The disclosure provides a method of treating a subject suffering from one or more missing, duplicated, aberrant, or aberrantly-spliced exons or missing or aberrant introns in the DMD gene in a cell, the method comprising administering to the subject an effective amount of a nucleic acid encoding a first DMD-targeting guide RNA (gRNA) targeting intron 40 and a nucleic acid encoding a second DMD-targeting gRNA targeting intron 55; or a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55; a nucleic acid comprising a donor DNA sequence encoding knock-in donor sequence of exons 41-55 the DMD gene flanked on each side of the donor sequences by a genomic Cas9 cut site; and a nucleic acid encoding a Cas9 enzyme or a functional fragment thereof. The disclosure also provides a method of treating a subject suffering from one or more missing, duplicated, aberrant, or aberrantly-spliced exons or missing or aberrant introns in the DMD gene in a cell, the method comprising administering to the subject an effective amount of a vector comprising a nucleic acid encoding a first DMD-targeting guide RNA (gRNA) targeting intron 40 and a nucleic acid encoding a second DMD-targeting gRNA targeting intron 55; or a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55; a nucleic acid comprising a donor DNA sequence encoding knock-in donor sequence of exons 41-55 the DMD gene flanked on each side of the donor sequences by a genomic Cas9 cut site; and a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6. In some aspects, the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117. In some aspects, the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9. In some aspects, the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120. In some aspects, the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188. In some aspects, expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the subject is a human subject. In some aspects, the human subject suffers from a muscular dystrophy. In some aspects, the muscular dystrophy is Duchene Muscular Dystrophy (DMD) or Becker Muscular Dystrophy (BMD).

The disclosure provides a recombinant gene editing complex comprising a nucleic acid encoding a first DMD-targeting guide RNA (gRNA) targeting intron 1 and a nucleic acid encoding a second DMD-targeting gRNA targeting intron 19; or a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19; a nucleic acid comprising a donor DNA sequence encoding knock-in donor sequence of exons 2-19 the DMD gene flanked on each side of the donor sequences by a genomic Cas9 cut site; and a nucleic acid encoding a Cas9 enzyme or a functional fragment thereof, wherein binding of the complex to the target nucleic acid sequence results in increased DMD gene expression. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28. In some aspects, the nucleic acid encoding a first DMD-targeting gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139. In some aspects, the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37. In some aspects, the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148. In some aspects, the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158. In some aspects, the nucleic acid encoding the gRNA or the nucleic acid encoding the Cas9 enzyme further comprises a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the one or more nucleic acids are in a vector. In some aspects, the vector is AAV.

The disclosure provides a recombinant gene editing complex comprising a nucleic acid encoding a first DMD-targeting guide RNA (gRNA) targeting intron 40 and a nucleic acid encoding a second DMD-targeting gRNA targeting intron 55; or a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55; a nucleic acid comprising a donor DNA sequence encoding knock-in donor sequence of exons 41-55 of the DMD gene flanked on each side of the donor sequences by a genomic Cas9 cut site; and a nucleic acid encoding a Cas9 enzyme or a functional fragment thereof, wherein binding of the complex to the target nucleic acid sequence results in increased DMD gene expression. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6. In some aspects, the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117. In some aspects, the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9. In some aspects, the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120. In some aspects, the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188. In some aspects, the nucleic acid encoding the gRNA or the nucleic acid encoding the Cas9 enzyme further comprises a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the one or more nucleic acids are in a vector. In some aspects, the vector is AAV. In some aspects, the adeno-associated virus lacks rep and cap genes. In some aspects, the adeno-associated virus is a recombinant AAV (rAAV) or a self-complementary AAV (scAAV). In some aspects, the AAV is AAV1, AAV2, AAV3, AAV4, AAV5, AAV6, AAV7, AAV8, AAV9, AV10, AV11, AV12, AV13, AAVanc80, or AAVrh.74. In some more particular aspects, the AAV is rAAV9.

The disclosure provides uses of a nucleic acid encoding a Duchenne muscular dystrophy (DMD) gene-targeting guide RNA (gRNA) comprising the nucleotide sequence set forth in any one of SEQ ID NOs: 1-37 or a variant thereof comprising at least or about 80% identity to the nucleotide sequence set forth in any one of SEQ ID NOs: 1-37; or a nucleotide sequence that specifically hybridizes to a target nucleic acid encoding DMD comprising the nucleotide sequence set forth in any one of SEQ ID NOs: 112-148. The disclosure provides uses of a nucleic acid comprising a donor DNA sequence encoding knock-in donor sequence of the DMD gene comprising the nucleotide sequence set forth in SEQ ID NO: 149, 152, 155, 158, 172, 176, 187, or 188 or a variant thereof comprising at least or about 80% identity to the nucleotide sequence set forth in SEQ ID NO: 149, 152, 155, 158, 172, 176, 187, or 188. In some aspects, these uses include, but are not limited to, a therapeutic in treating one or more missing, duplicated, aberrant, or aberrantly-spliced exons or missing or aberrant introns in the DMD gene in a cell. In some aspects, the therapeutic is a medicament. In some aspects, the medicament is useful for treating one or more missing, duplicated, aberrant, or aberrantly-spliced exons or missing or aberrant introns in the DMD gene in a cell of a human subject.

The disclosure provides a method of increasing expression of the DMD gene or increasing the expression of a functional dystrophin in a cell, wherein the method comprises contacting the cell with: (a) a nucleic acid encoding a DMD-targeting guide RNA (gRNA) targeting intron 19; or a nucleic acid encoding a DMD-targeting gRNA that specifically hybridizes to a target nucleotide sequence in intron 19; (b) a nucleic acid comprising a donor DNA sequence encoding knock-in donor sequence of exons 1-19 the DMD gene flanked on each side of the donor sequences by a genomic Cas9 cut site; and (c) a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding the DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37. In some aspects, the nucleic acid encoding the DMD-targeting gRNA comprises a nucleotide sequence that specifically hybridizes to the target sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148. In some aspects, the nucleic acid encoding the knock-in donor sequence of exons 1-19 comprises a nucleotide sequence selected from the group consisting of: (a) the nucleotide sequence set forth in SEQ ID NO: 173 or 178 or a variant thereof comprising at least or about 80% identity to the nucleotide sequence set forth in SEQ ID NO: 173 or 178; (b) the nucleotide sequence set forth in SEQ ID NO: 174 or a variant thereof comprising at least or about 80% identity to the nucleotide sequence set forth in SEQ ID NO: 174; (c) the nucleotide sequence set forth in SEQ ID NO: 175 or a variant thereof comprising at least or about 80% identity to the nucleotide sequence set forth in SEQ ID NO: 175; (d) the nucleotide sequence set forth in SEQ ID NO: 176 or a variant thereof comprising at least or about 80% identity to the nucleotide sequence set forth in SEQ ID NO: 176; and (e) the nucleotide sequence set forth in SEQ ID NO: 177 or a variant thereof comprising at least or about 80% identity to the nucleotide sequence set forth in SEQ ID NO: 177. In some aspects, the nucleic acid encoding the knock-in donor sequence of exons 1-19 comprises the nucleotide sequence set forth in SEQ ID NO: 172 or a variant thereof comprising at least or about 80% identity to the nucleotide sequence set forth in SEQ ID NO: 172. In some aspects, expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the nucleic acid is in a vector. In some aspects, the vector is AAV. In some aspects, the subject is a human subject. In some aspects, the human subject suffers from a muscular dystrophy. In some aspects, the muscular dystrophy is Duchene Muscular Dystrophy (DMD) or Becker Muscular Dystrophy (BMD).

The disclosure provides a method of treating a subject suffering from one or more missing, duplicated, aberrant, or aberrantly-spliced exons or missing or aberrant introns in the DMD gene in a cell, the method comprising administering to the subject an effective amount of: (a) a nucleic acid encoding a DMD-targeting guide RNA (gRNA) targeting intron 19; or a nucleic acid encoding a DMD-targeting gRNA that specifically hybridizes to a target nucleotide sequence in intron 19; (b) a nucleic acid comprising a donor DNA sequence encoding knock-in donor sequence of exons 1-19 the DMD gene flanked on each side of the donor sequences by a genomic Cas9 cut site; and (c) a nucleic acid encoding a Cas9 enzyme or a functional fragment thereof. In some aspects, the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof. In some aspects, the nucleic acid encoding the DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37. In some aspects, the nucleic acid encoding the DMD-targeting gRNA comprises a nucleotide sequence that specifically hybridizes to the target sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148. In some aspects, the nucleic acid encoding the knock-in donor sequence of exons 1-19 comprises a nucleotide sequence selected from the group consisting of: (a) the nucleotide sequence set forth in SEQ ID NO: 173 or 178 or a variant thereof comprising at least or about 80% identity to the nucleotide sequence set forth in SEQ ID NO: 173 or 178; (b) the nucleotide sequence set forth in SEQ ID NO: 174 or a variant thereof comprising at least or about 80% identity to the nucleotide sequence set forth in SEQ ID NO: 174; (c) the nucleotide sequence set forth in SEQ ID NO: 175 or a variant thereof comprising at least or about 80% identity to the nucleotide sequence set forth in SEQ ID NO: 175; (d) the nucleotide sequence set forth in SEQ ID NO: 176 or a variant thereof comprising at least or about 80% identity to the nucleotide sequence set forth in SEQ ID NO: 176; and (e) the nucleotide sequence set forth in SEQ ID NO: 177 or a variant thereof comprising at least or about 80% identity to the nucleotide sequence set forth in SEQ ID NO: 177. In some aspects, the nucleic acid encoding the knock-in donor sequence of exons 1-19 comprises the nucleotide sequence set forth in SEQ ID NO: 172 or a variant thereof comprising at least or about 80% identity to the nucleotide sequence set forth in SEQ ID NO: 172. In some aspects, expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter. In some aspects, the nucleic acid is in a vector. In some aspects, the vector is AAV. In some aspects, the subject is a human subject. In some aspects, the human subject suffers from a muscular dystrophy. In some aspects, the muscular dystrophy is Duchene Muscular Dystrophy (DMD) or Becker Muscular Dystrophy (BMD).

The disclosure also provides a nucleic acid encoding a CRISPR-associated (Cas) enzyme comprising at its 5′ end a polynucleotide encoding a nuclear localization signal comprising a nucleotide sequence comprising the nucleotide sequence set out in SEQ ID NO: 179 or a variant thereof comprising at least or about 70% identity to the nucleotide sequence set out in SEQ ID NO: 179; or a nucleotide sequence encoding the amino acid sequence set out in SEQ ID NO: 180 or a variant thereof comprising at least or about 70% identity to amino acid sequence set out in SEQ ID NO: 180. In some aspects, the Cas enzyme is Cas9 or Cas13.

Further aspects and advantages of the disclosure will be apparent to those of ordinary skill in the art from a review of the following detailed description, taken in conjunction with the drawings. It should be understood, however, that the detailed description (including the drawings and the specific examples), while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIG. 1A-C depicts properties of dystrophin. FIG. 1A shows a schematic of the axis of force transduction in muscle cells. Dystrophin links the cytoskeletal actin to the transmembrane dystroglycan complex thus linking the cytoskeleton to the extracellular matrix via laminin. FIG. 1B shows a schematic of the dystrophin protein with the major domains labeled. FIG. 1C shows a schematic of the DMD gene diagram of exons corresponding to each domain in dystrophin. The shape of each exon depicts reading frame phasing, while exons encircled by red boxes show mutation hotspots within the DMD gene (e.g., exons 6-7, 43-46, and 50-53).

FIG. 2 shows a depiction of Cas9 targeting via the use of a gRNA complementary to a portion of genomic DNA. The genomic DNA bound by the gRNA is shown in red. In blue is the PAM site. The SaCas9 protein is shown in orange.

FIG. 3A-B is a schematic showing the HITI strategy for (FIG. 3A) exon 2 replacement and (FIG. 3B) exon 2+3 replacement. Proper cleavage at the two genomic loci and of the knock in fragment can result in one of three possible outcomes. Simple deletion of the flanked exon(s), inverse integration of the knock in fragment, or proper forward knock in. Inverse knock in results in reconstitution of the cut sites allowing for re-cleavage.

FIG. 4A-B provide gel images showing successful knock in of the HITI donor following PCR of treated HEK293 genomic DNA with knock-in specific primers. This was shown for both (FIG. 4A) exon 2 replacement and (FIG. 4B) exon 2-3 replacement. Sequencing data showed that there was perfect joining of (FIG. 4C) the 5′ end of the HITI insertion as well as (FIG. 4D) the 3 end of the HITI insertion. n=3 biological replicates.

FIG. 5A-B shows gel images depicting the CRISPR:Donor titration experiments. Results from both (FIG. 5A) increasing the Donor amount compared to the CRISPR amount and (FIG. 5B) increasing the CRISPR amount compared to the Donor amount. These experiments indicated a ratio of 1:1 is optimal for this triple plasmid co-transfected system.

FIG. 6A-B shows homology models of predicted structure of (FIG. 6A) the endogenous spectrin-like repeat 22 and (FIG. 6B) the hybrid spectrin-like repeat produced from joining of exons 40 and 56 built using SWISS-MODEL. Blue areas depict more favorable global quality estimates whereas red areas depict less favorable global quality estimates.

FIG. 7A-B shows a depiction of the plasmids used for the exon 41-55 HITI replacement strategy (FIG. 7A) and a schematic showing the editing outcomes affiliated with this HITI strategy (FIG. 7B). Red boxes surrounding exons 43-46 and 50-53 show mutational hotspots within the DMD gene. Note that the DMD exon 41-55 CDS is flanked by 100 bp of the native or synthetic adjacent introns to ensure splicing into transcripts.

FIG. 8A-B shows polyacrylamide gel images of the T7E1 assay (EnGen® Mutation Detection Kit; New England Biolabs) for the (FIG. 8A) JHI55A targeting series and (FIG. 8B) the JHI40 targeting series. “Unt.” denotes untreated DNA and “cont.” denotes the positive control provided with the EnGen® Mutation Detection Kit (New England Biolabs). Yellow dots denote the expected band sizes with editing at the respective target site. “+” and “−” denotes reactions with and without T7E1 enzyme, respectively. Active gRNAs are denoted in green text.

FIG. 9 shows fluorescence microscopy images showing co-transfections of the two HITI plasmids with the percentage of double-positive cells compared to the total amount of counted cells.

FIG. 10A-B show results for knocking in the HITI donor sequence. FIG. 10A shows a gel image showing the successful knock in of the HITI donor at the 41-55 site using a 5′ knock-in specific primer set. FIG. 10B shows a Sanger sequencing chromatogram depicting seamless integration of the HITI donor sequence based on the highlighted junction. n=3 biological replicates.

FIG. 11 shows a schematic approach to DMD gene correction using HITI with three likely gene editing outcomes. Arrows indicate directionality of genetic elements and expression cassettes. Note that the DMD exon 41-55 CDS is flanked by 100 bp of the native or synthetic adjacent introns to ensure splicing into transcripts.

FIG. 12 provides a gel image showing HITI knock-in of a GFP cassette in place of DMD exon 2 in HEK293 cells. Primer locations indicated beside each gel image. “Non-donor” is a plasmid with the GFP cassette lacking Cas9 cut sites. n=3 biological replicates.

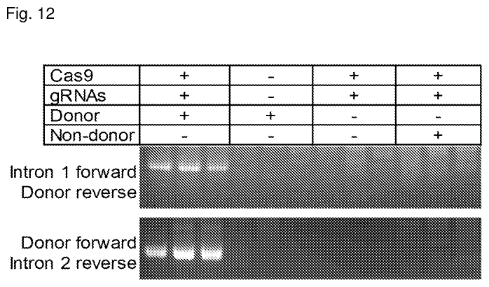

FIG. 13 provides a gel image showing HITI knock-in of a GFP cassette in place of DMD exons 2-3 in HEK293 cells. Primer locations indicated beside each gel image. “Non-donor” is a plasmid with the GFP cassette lacking Cas9 cut sites. n=3 biological replicates.

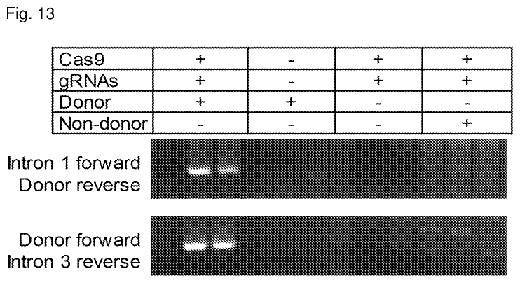

FIG. 14 provides gel images of HITI knock-in of DMD exons 2-19 in place of the natural DMD exon 2-19 locus in HEK293 cells 72 hours after transfection with (+) or without (−) plasmids encoding i) CMVP-driven SaCas9 and ii) a HITI donor sequence encoding DMD exons 2-19 and synthetic splice sites (as in Seq ID No: 155) as well as U6-promoter driven gRNAs DSAi1-03 and DSAi19-004. Forward (F) and reverse (R) primer annealing locations indicated beside each gel image. n=3 biological replicates.

FIG. 15A-B provides data from a successful knock in of a plasmid-derived DMD exon 41-55 CDS in place of the natural DMD exon 41-55 locus in HEK293 cells. FIG. 15A provides a gel image of genomic DNA PCR amplicons corresponding to successful knock in of a plasmid-derived DMD exon 41-55 CDS in place of the natural DMD exon 41-55 locus in HEK293 cells with primer annealing sites indicated. M is a DNA size marker. n=3 biological replicates. FIG. 15B provides Sanger sequencing of the knock-in amplicon from panel FIG. 15A. Native or synthetic intronic sequence upstream of the targeted locus and knock-in derived sequences highlighted in blue and yellow, respectively. Black arrow indicates the Cas9 target site ablated by the desired HITI knock-in.

FIG. 16 shows TIDE quantitation of gene editing in HEK293 cells at the respective target sites of gRNAs as indicated below each bar. Values reflect averages with standard deviation error bars, n=3 biological replicates, unless otherwise indicated. Samples indicated by an asterisk resulted in poor DNA sequencing reads and were not analyzed.

FIG. 17 depicts the strategy for knock in of an MHCK7 promoter followed by DMD exons 1-19 into intron 19 as well as the potential outcomes affiliated with this HITI strategy. DMD exons 1-19 CDS is followed by a splice donor sequence to ensure splicing into transcripts.

FIG. 18 shows a gel image of RT-PCR amplicons from del45 patient-derived cells with (treated) and without (untreated) treatment using AAV1s encoding Sa Cas9, U6-driven gRNAs JHI40-008 and JHI55A-004, as well as a donor DNA sequence encoding DMD exons 41-55 flanked by splice sites and bookended by JHI40-008 and JHI55A-004 target sites. Primers annealed to DMD exon 43 and exon 46. Untreated cells have a smaller amplicon than wild-type due to deletion of exon 45. Replacement of the defective exons 41-55 locus in the patient cells with the mega-exon within the HITI donor sequence results in a larger amplicon corresponding to wild-type. n=3 biological replicates.

### DETAILED DESCRIPTION

The disclosure provides products, methods, and uses for treating, ameliorating, delaying the progression of, and/or preventing a muscular dystrophy involving a mutation involving, surrounding, or affecting large regions of the DMD gene encompassing multiple DMD exons.

The products and methods provided herein provide an altered form of dystrophin protein for use in treating a muscular dystrophy resulting from a mutation involving, surrounding, or affecting various regions of the DMD gene. DMD, the largest known human gene, provides instructions for making a protein called dystrophin. Dystrophin is located primarily in muscles used for movement (skeletal muscles) and in heart (cardiac) muscle. In some aspects, the mutation is involving, surrounding, or affecting the DMD locus in a region encompassed by introns 1-19 and introns 40-55. In some aspects, the mutation is involving, surrounding, or affecting DMD exons 1-19, 2-19, or 41-55. In some aspects, the mutation is encompassed by the DMD promoter, the 5′ untranslated region, as well as exon 1 through intron 19.

The mutations included for treatment by the products, methods and uses of the disclosure include, but are not limited to, mutations or rearrangements, such as large and small duplications, deletions, single nucleotide polymorphisms (SNPs), or other mutations. In some aspects, the disclosure provides products, methods and uses for treating, ameliorating, delaying the progression of, and/or preventing a muscular dystrophy involving a mutation involving, surrounding, or affecting the DMD locus in a region encompassed by introns 1-19 (or involving, surrounding, or affecting exons 2-19 of the DMD gene), introns 40-55 (or involving, surrounding, or affecting exons 41-55 of the DMD gene), the DMD promoter (i.e., DMD Dp427m promoter), the 5′ untranslated region, or exon 1 through intron 19. In some aspects, the mutation is involving, surrounding, or affecting DMD exons 1-19, 2-19, or 41-55. In some aspects, the mutation is encompassed by the DMD promoter, the 5′ untranslated region, as well as exon 1 through intron 19.

In some aspects, the disclosure provides products, methods and uses for treating or ameliorating a complete or partial duplication or deletion of one or more exons within the exon 2-19 locus; an insertion or deletion of one or more base pairs in any one or more of exons 2-19; a nonsense or missense point mutation in any one or more of exons 2-19; or an insertion, deletion, duplication, or point mutation within any one or spanning multiple of introns 1-19 that affects proper splicing or translation of any one or more of exons 2-19. In some aspects, the disclosure provides products, methods and uses for treating or ameliorating a complete or partial duplication or deletion of one or more exons within the exon 41-55 locus; an insertion or deletion of one or more base pairs in any one or more of exons 41-55; a nonsense or missense point mutation in any one or more of exons 41-55; or an insertion, deletion, duplication, or point mutation within any one or spanning multiple of introns 40-55 that affects proper splicing or translation of any one or more of exons 41-55.

In some aspects, the mutation is involving, surrounding, or affecting exons 2-19 of the DMD gene, or a region encompassed by introns 1-19 including, but not limited to, a deletion of exon 3, a deletion of exons 3-7, a deletion of exons 3-11, a deletion of exon 7, a deletion of exons 8-9, a deletion of exons 8-11, a deletion of exons 8-13, a deletion of exons 10-11, a deletion of exon 18, a duplication of DMD exon 2, a duplication of exons 2-6, a duplication of exons 2-7, a duplication of exons 2-19, a duplication of DMD exons 2-11, a duplication of exons 3-4, a duplication of exons 3-7, a duplication of exons 5-7, a duplication of exons 8-9, a duplication of exons 8-11, a duplication of exons 8-13, a duplication of exon 12, a duplication of exons 12-13, a duplication of exon 18, a duplication of exon 19, a nonsense point mutation in exon 6, a nonsense point mutation in exon 7, a nonsense point mutation in exon 8, a nonsense point mutation in exon 9, a nonsense point mutation in exon 10, a nonsense point mutation in exon 11, a nonsense point mutation in exon 12, a nonsense point mutation in exon 13, a nonsense point mutation in exon 14, a nonsense point mutation in exon 15, a nonsense point mutation in exon 16, a nonsense point mutation in exon 17, a nonsense point mutation in exon 18, or a nonsense point mutation in exon 19, a frameshifting insertion or deletion mutation in exon 6, a frameshifting insertion or deletion mutation in exon 7, a frameshifting insertion or deletion mutation in exon 8, a frameshifting insertion or deletion mutation in exon 9, a frameshifting insertion or deletion mutation in exon 10, a frameshifting insertion or deletion mutation in exon 11, a frameshifting insertion or deletion mutation in exon 12, a frameshifting insertion or deletion mutation in exon 13, a frameshifting insertion or deletion mutation in exon 14, a frameshifting insertion or deletion mutation in exon 15, a frameshifting insertion or deletion mutation in exon 16, a frameshifting insertion or deletion mutation in exon 17, a frameshifting insertion or deletion mutation in exon 18, or frameshifting insertion or deletion mutation in exon 19.

In some aspects, the mutation is involving, surrounding, or affecting DMD exons 1-19, or a region encompassed by the DMD promoter, the 5′untranslated region, as well as exon 1 through intron 19 including, but not limited to, a deletion of exon 3, a deletion of exons 3-7, a deletion of exons 3-11, a deletion of exon 7, a deletion of exons 8-9, a deletion of exons 8-11, a deletion of exons 8-13, a deletion of exons 10-11, a deletion of exon 18, a duplication of DMD exon 2, a duplication of exons 2-6, a duplication of exons 2-7, a duplication of exons 2-19, a duplication of DMD exons 2-11, a duplication of exons 3-4, a duplication of exons 3-7, a duplication of exons 5-7, a duplication of exons 8-9, a duplication of exons 8-11, a duplication of exons 8-13, a duplication of exon 12, a duplication of exons 12-13, a duplication of exon 18, a duplication of exon 19, a nonsense point mutation in exon 6, a nonsense point mutation in exon 7, a nonsense point mutation in exon 8, a nonsense point mutation in exon 9, a nonsense point mutation in exon 10, a nonsense point mutation in exon 11, a nonsense point mutation in exon 12, a nonsense point mutation in exon 13, a nonsense point mutation in exon 14, a nonsense point mutation in exon 15, a nonsense point mutation in exon 16, a nonsense point mutation in exon 17, a nonsense point mutation in exon 18, or a nonsense point mutation in exon 19, a frameshifting insertion or deletion mutation in exon 6, a frameshifting insertion or deletion mutation in exon 7, a frameshifting insertion or deletion mutation in exon 8, a frameshifting insertion or deletion mutation in exon 9, a frameshifting insertion or deletion mutation in exon 10, a frameshifting insertion or deletion mutation in exon 11, a frameshifting insertion or deletion mutation in exon 12, a frameshifting insertion or deletion mutation in exon 13, a frameshifting insertion or deletion mutation in exon 14, a frameshifting insertion or deletion mutation in exon 15, a frameshifting insertion or deletion mutation in exon 16, a frameshifting insertion or deletion mutation in exon 17, a frameshifting insertion or deletion mutation in exon 18, or frameshifting insertion or deletion mutation in exon 19.

In some aspects, the mutation is involving, surrounding, or affecting exons 41-55 of the DMD gene, or a region encompassed by introns 40-55 including, but not limited to, a duplication of DMD exon 44, a deletion of exon 43, 44, 45, 46, 48, 49, 50, 51, 52, or 53, a deletion of exons 45-50, a deletion of exons 45-52, a deletion of exons 45-54, a deletion of exons 46-47, a deletion of exons 46-48, a deletion of exons 46-50, a deletion of exons 46-51, a deletion of exons 46-52, a deletion of exons 48-50, a deletion of exons 48-54, a deletion of exons 49-50, a deletion of exons 49-52, a deletion of exons 49-54, a deletion of exons 50-52, a deletion of exons 52-54, a deletion of exons 53-54, a duplication of exons 42-43, a duplication of exon 43, a duplication of exon 44, a duplication of exons 44-51, a duplication of exon 45, a duplication of exon 46, a duplication of exons 46-47, a duplication of exon 53, a nonsense point mutation in exon 41, a nonsense point mutation in exon42, a nonsense point mutation in exon43, a nonsense point mutation in exon44, a nonsense point mutation in exon 45, a nonsense point mutation in exon 46, a nonsense point mutation in exon 47, a nonsense point mutation in exon 48, a nonsense point mutation in exon 49, a nonsense point mutation in exon 50, a nonsense point mutation in exon 51, a nonsense point mutation in exon 52, a nonsense point mutation in exon 53, a nonsense point mutation in exon 54, a nonsense point mutation in exon 55, a frameshifting insertion or deletion mutation in exon 41, a frameshifting insertion or deletion mutation in exon 42, a frameshifting insertion or deletion mutation in exon 43, a frameshifting insertion or deletion mutation in exon 44, a frameshifting insertion or deletion mutation in exon 45, a frameshifting insertion or deletion mutation in exon 46, a frameshifting insertion or deletion mutation in exon 47, a frameshifting insertion or deletion mutation in exon 48, a frameshifting insertion or deletion mutation in exon 49, a frameshifting insertion or deletion mutation in exon 50, a frameshifting insertion or deletion mutation in exon 51, a frameshifting insertion or deletion mutation in exon 52, a frameshifting insertion or deletion mutation in exon 53, a frameshifting insertion or deletion mutation in exon 54, or a frameshifting insertion or deletion mutation in exon 55.

More particularly, the disclosure provides nucleic acids comprising nucleotide sequences encoding guide RNAs (gRNAs), nucleic acids comprising nucleotide sequences encoding multiple exons of the DMD gene to be knocked-in with homology-independent targeted insertion (HITI), a CRISPR/Cas9-based strategy to induce DNA knock-in or make large replacements of genomic DMD DNA, and vectors comprising the nucleic acids for carrying out the HITI knock-ins of the various DMD regions. The disclosure therefore provides products, methods, and uses for restoring functional dystrophin to a vast cohort of muscular dystrophy patients with diverse mutations of the DMD gene.

Dystrophin and Duchenne Muscular Dystrophy

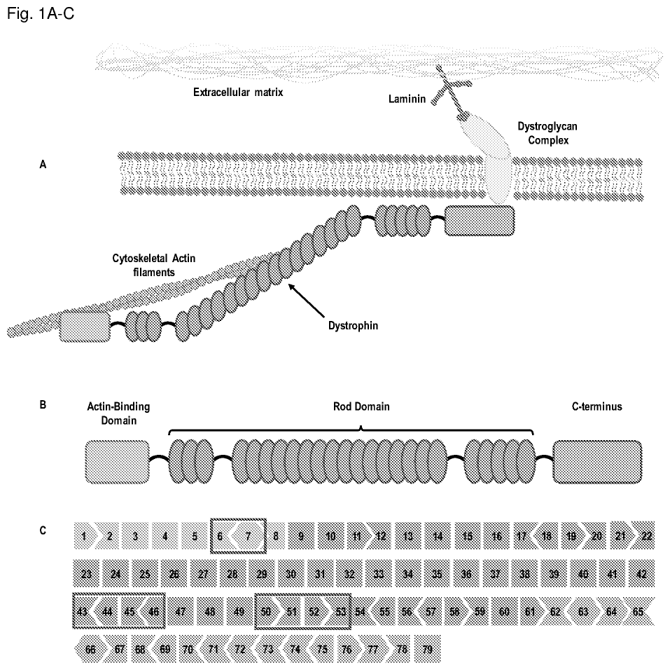

The disclosure provides products, methods and uses for treating a muscular dystrophy resulting from a mutation in the DMD gene. Such muscular dystrophies include, but are not limited to, Duchenne muscular dystrophy (DMD) and Becker muscular dystrophy (BMD). DMD is an X-linked genetic disorder caused by myriad mutations within the DMD gene which contains a total of 79 exons and codes for the 427 kDa muscle isoform of the dystrophin protein (FIG. 1A-C) (Flanigan, Neurol Clin 32, 671-688, viii, doi:10.1016/j.ncl.2014.05.002 (2014)). The DMD gene encodes the dystrophin protein, which is one of the longest human genes known. Dystrophin is a structural protein which serves to reinforce the plasma membrane via a connection between cytoskeletal actin filaments and the dystroglycan complex (DGC) (FIG. 1A) (Gao et al., Compr Physiol 5, 1223-1239, doi:10.1002/cphy.c140048 (2015)). As such, dystrophin has several key domains including an N-terminal actin binding domain, a central rod domain comprised of spectrin-like repeats with a second actin binding domain, and a C-terminal domain that directly interacts with the DGC (FIG. 1B) (Gao et al., supra). Dystrophin acts as a shock-absorber during normal muscle contraction and is required to prevent muscle damage and degeneration during normal activity. In the absence of dystrophin, muscle degeneration leads to weakness which eventually progresses to a loss of ambulation in the early teens.1 Once in a wheelchair, patients have steep declines in cardiac and respiratory function (due to the involvement of the heart and diaphragm, respectively) which are the primary causes of the early mortality characteristic of DMD.

The DMD gene, the gene encoding the dystrophin protein, has a diverse mutational profile, due in part to the size of the gene (Bladen et al., Hum Mutat 36, 395-402, doi:10.1002/humu.22758 (2015)). Single nucleotide point mutations, which are the result of single base pair changes in the DNA sequence, account for about 10.5% of DMD causing mutations (Bladen et al. supra). Exonic duplications account for about 10.9% of DMD mutations and occur when a portion of the gene is duplicated and placed directly adjacent to the original gene fragment (Bladen et al. supra). Exonic deletions are when a portion of the gene containing one or more exons is fully excised from the gene, and account for about 68.5% of DMD mutations (Bladen et al. supra). Both exonic deletions and duplications usually result in frameshift mutations that generally lead to loss of functional dystrophin protein. Another 6.9% of DMD mutations consist of subexonic insertions and deletions (indels) that also generally result in frameshift mutations (Bladen et al. supra). Another 2.7% of DMD mutations consist of mutations that affect the splice sites of certain exons (Bladen et al. supra). The final 0.5% of mutations consist of variable and highly specific mutations throughout the intronic regions of the DMD gene (Bladen et al. supra). Despite this extensive mutational profile, gene editing has shown great potential in correcting many of the types of mutations described above.

CRISPR/Cas9 Gene Editing

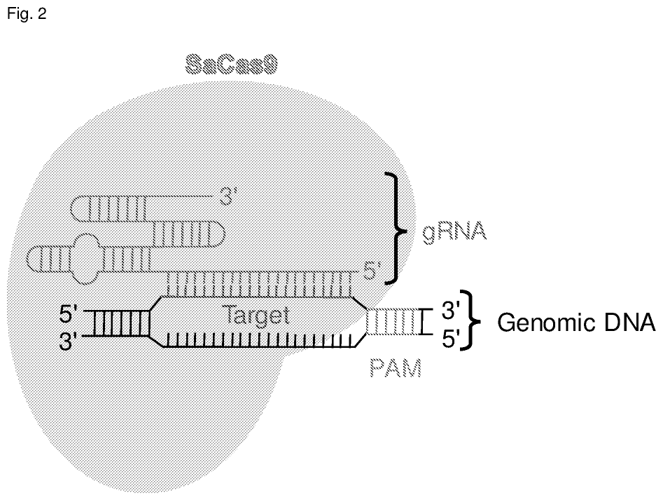

Clustered Regularly Interspaced Short Palindromic Repeats and the associated protein 9 (“CRISPR-associated protein 9” or “CRISPR/Cas9”) is an adaptive immune system found in bacteria that utilizes an RNA-programmable endonuclease to protect bacteria against viral invaders. This system, which consists of a guide RNA (gRNA) and a Cas9 endonuclease protein, has been repurposed to make precise double stranded breaks (DSBs) at a site complementary to the gRNA and near a short recognition sequence known as a protospacer adjacent motif (PAM) site (FIG. 2). Cas9 (CRISPR associated protein 9, formerly called Cas5, Csn1, or Csx12) is a 160 kilo Dalton protein which plays a vital role in the immunological defense of certain bacteria against DNA viruses and plasmids and which is heavily utilized in genetic engineering applications. Cas9 is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms (Zhang et al. (2014) Human Molecular Genetics. 23 (R1): R40-6. doi:10.1093/hmg/ddu125. PMID 24651067). This editing process has a wide variety of applications including basic biological research, development of biotechnology products, and treatment of diseases.

The disclosure utilizes Cas9 in the gene editing complex, methods and uses disclosed herein. The disclosure included the use of all species, homologs, and variants of Cas9, including functional fragments thereof. There are several different homologs of the Cas9 protein from different bacteria which have differences in size and PAM recognition sequence. The most well characterized variant is Cas9 from Streptococcus pyogenes (SpCas9) which is encoded by 1,371 amino acids and has a PAM recognition sequence of 5′-NGG-3′ (Jinek et al., Science 337, 816-821, doi:10.1126/science.1225829 (2012); Ran et al., Nat Protoc 8, 2281-2308, doi:10.1038/nprot.2013.143 (2013); Zhang et al., Physiol Rev 98, 1205-1240, doi:10.1152/physrev.00046.2017 (2018)). A less commonly used Cas protein is from Staphylococcus aureus (SaCas9) which, in contrast to SpCas9, is encoded by 1,053 amino acids and has a PAM recognition sequence of 5′-NNGRRT-3′ (SEQ ID NO: 163) (Ran et al., Nature 520, 186-191, doi:10.1038/nature14299 (2015)). The use of the smaller SaCas9 protein is preferable, in some aspects, in virally delivered gene therapies on account of the limited cargo space (˜5 kb) associated with viral vectors such as the Adeno-Associated Virus (AAV) (Grieger et al., J Virol 79, 9933-9944, doi:10.1128/JVI.79.15.9933-9944.2005 (2005)). Nevertheless, the disclosure includes the use of all various species, homologs, and variants of Cas9, and is not limited to the particular Cas9 exemplified herein. In exemplary aspects, the disclosure provides the nucleotide sequences encoding S. aureus Cas9 (SEQ ID NO: 161) or S. aureus Cas9 with a nuclear localization signal (SEQ ID NO: 181) and C. jejuni Cas9 (SEQ ID NO: 162) or C. jejuni Cas9 with a nuclear localization signal (SEQ ID NO: 183).

The disclosure includes CRISPR/Cas9, Cas 9 homologs, Cas9 orthologs, and Cas9 variants, including engineered Cas9 variants, and methods of using said CRISPR/Cas9, Cas 9 homologs, Cas9 orthologs, and Cas9 variants, including engineered Cas9 variants (e.g., Liu et al., Nat Commun 11, 3576 (2020); WO 2014/191521) and split-Cas9 (e.g., WO 2016/112242; WO 2017/197238). As used herein, the term “Cas9” is any species, homolog, ortholog, engineered, or variant of Cas9, including split-Cas9, or a functional fragment thereof. There are several different homologs of the Cas9 protein from different bacteria which have differences in size and PAM recognition sequence. In various exemplary aspects of the disclosure, Staphylococcus aureus (SaCas9) and Campylobacter jejuni Cas9 (CjCas9) are provided. The disclosure is not limited to these particular species of Cas9. In some aspects, the Cas9 is mammalian codon optimized. In some aspects, e.g., the SaCas9 is described by Tan et al. (PNAS Oct. 15, 2019 116 (42) 20969-20976; https://doi.org/10.1073/pnas.1906843116). In some aspects, the Campylobacter jejuni Cas9 is commercially available, e.g., PX404 from Addgene (Cat. No. 68338, https://www.addgene.org/68338/sequences/). In some aspects, the SpCas9 is described in the literature (UniProtKB—Q1JH43 (Q1JH43_STRPD). In some aspects, the Cas9 is modified with a nuclear localization signal (e.g., as set out in SEQ ID NO: 181 or 183).

Homology Independent Targeted Integration

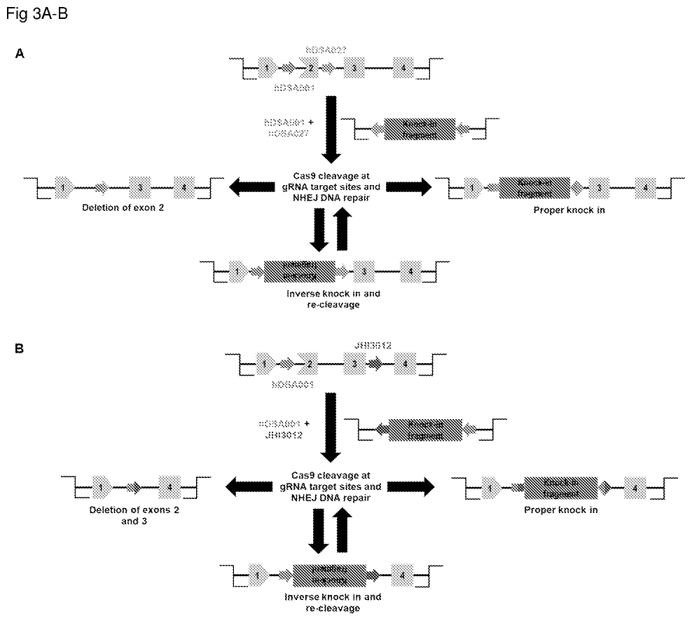

The disclosure utilizes Homology-Independent Targeted-Integration (HITI) to accomplish high efficiency knock in using the non-homologous end-joining (NHEJ) DNA repair pathway (Suzuki et al., Nature 540, 144-149, doi:10.1038/nature20565 (2016); Zare et al., Biol Proced Online. 20:21 (2018) doi:10.1186/s12575-018-0086-5; Roman-Rodriguez et al., Cell Stem Cell. 25(5):607-21(2019)). HITI requires two components; i) CRISPR/Cas9 and ii) a donor DNA containing CRISPR/Cas9 cut sites flanking the desired knock-in fragment (FIG. 3) (Suzuki, supra). CRISPR/Cas9 generates a genomic DNA break while also cleaving the donor DNA and activating it as a NHEJ substrate for integration into the genome DSB. HITI was initially designed with a single target site and has been utilized to knock in a missing exon in Mertk, the gene implicated in a rat model of retinitis pigmentosa (Suzuki, supra). The HITI treated rats showed improved eye function when compared to their untreated or HDR treated counterparts (Suzuki, supra). Suzuki's study used mice to show that the in vivo efficiency of systemic AAV-mediated HITI knock-in of a reporter gene to be ˜10% in the quadriceps muscle and ˜3-4% in heart muscle (Suzuki, supra). Gene editing using HITI also has been described in International Patent Publication No. WO 2017/197238, incorporated herein by reference in its entirety.

The disclosure utilizes HITI-based gene editing strategy to replace missing, duplicated, aberrant, or aberrantly-spliced exons, or missing or aberrant introns in the DMD gene. The HITI-based gene editing strategy disclosed herein was designed to enable the restoration of dystrophin, including full-length dystrophin, in patients suffering from a deficiency in dystrophin. Advantages of this gene editing approach over current therapies include the restoration of dystrophin protein, and in some aspects a full-length dystrophin protein, which is an attractive concept due to the known long-term consequences of a truncated isoform of dystrophin on the functional outcomes of those affected by DMD. Since this approach corrects dystrophin at the genomic level, there is potential for this therapy to be a one-time administration as opposed to therapies that require life-long dosing to have the desired effect. A further advantage of this therapy over other therapies that aim at genomic correction is the range of patient cohort that would benefit. Rather than being an approach targeted at a specific mutation, the disclosure provides a method to effectively correct any mutation within larger target regions of the DMD gene including, but not limited to, a mutation is involving, surrounding, or affecting the DMD locus in a region encompassed by introns 1-19 and introns 40-55. In some aspects, the mutation is involving, surrounding, or affecting DMD exons 1-19, 2-19, or 41-55 of the DMD gene. In some aspects, the mutation is encompassed by the DMD Dp427m promoter, the 5′ untranslated region, as well as exon 1 through intron 19. Although others have published on replacement of small regions of the DMD gene (i.e., US2016/0201089; US2019/0134221), the disclosure is directed to products and methods of replacing large regions of the DMD gene covering over 15 exons using a HITI-based gene editing strategy.

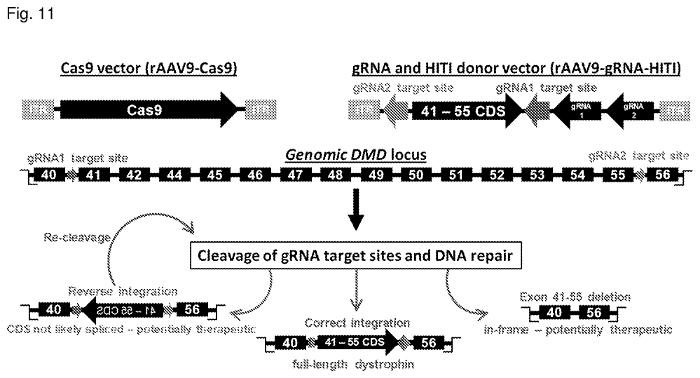

HITI includes three components: i) Cas9 to generate DNA double-stranded breaks at user-chosen sites on the gene of interest, i.e., the DMD gene, ii) guide RNAs (gRNAs) to guide Cas9 to user-chosen DNA sites, and iii) a donor DNA containing the desired knock-in sequence flanked by one or more of the gRNA target sites (FIG. 11). Importantly, HITI uses the NHEJ DNA repair pathway which can result in many potential repair outcomes including i) rejoining of the DNA ends without donor integration, ii) correct integration of the donor, and iii) inverted donor integration (FIG. 11). To improve the likelihood of correct donor integration, the target sites within the HITI donor DNA are engineered as reverse compliments of the genomic target sites, such that reverse integration reconstitutes these target sites and allows additional rounds of cleavage by Cas9 and potential knock-in by NHEJ (FIG. 11). Upon correct integration, the target sites are ablated, thus preventing further cleavage by Cas9. For replacement of exons 41-55, deletion of exons 41-55 without donor integration results in a coherent reading frame and potentially leads to expression of a Becker-like dystrophin isoform (FIG. 11) thus increasing the therapeutic potential of this outcome.

Guide RNAs, Target Sites, and Donor DNAs

The disclosure includes guide RNAs (gRNAs) to guide Cas9 to user-chosen DNA sites, target sites on the DMD gene for guide RNA targeting, donor DNA containing the desired knock-in sequence flanked by one or more of the gRNA target sites, and Cas9 to generate DNA double-stranded breaks at user-chosen sites on the DMD gene.

The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence.

As used herein, “target” or ‘target sequence” or “target nucleic acid” is either the forward or reverse strand of the sequences provided herein designated as target sequence. Thus, the target nucleic acid, as recited in the claims is the coding strand or its complement. For example, in Tables 1 and 3, the target sequence is provided as the coding strand of the DMD gene. In Tables 2 and 4, the complete sequence is given as the coding strand; however, the gRNA target sites have been changed to the reverse complement of the coding strand sequence, which is required for HITI to accomplish high efficiency knock in using the non-homologous end-joining (NHEJ) DNA repair pathway.

Table 1 provided herein below provides Staphylococcus aureus gRNAs that target human DMD introns 40 or 55, including full gRNA sequences, spacer sequences of the gRNAs, the direct repeat tracrRNA sequence, and the target sequence to which the guide RNA is designed to target. The gRNAs are not designed to bind only coding or only non-coding strands of the DMD gene; some bind one and others bind the other strand. Cas9 requires double-stranded DNA to bind and cut; however, the gRNA anneals to only one of the two strands. Despite this, Cas9 binds and cuts both strands of the given sequences. The natural CRISPR Cas9 system contains two RNAs, one is called the crRNA and contains sequences called spacer (assigns its targeting specificity) direct repeat (helps it bind with tracrRNA and Cas9) and a tracrRNA which contains a region complementary to the crRNA direct repeat and anneals to the crRNA direct repeat sequence such that they form a dsRNA that binds to Cas9. Guide RNAs can target either the coding or non-coding strand. The strand a gRNA should be designed to bind depends on which strand the PAM sequence is on. The strand that contains the PAM (e.g., 5′-NNGRRT-3′ for SaCas9) is called the non-target strand and it contains the protospacer sequence which matches the sequence of the corresponding spacer region of the gRNA. The spacer region of the gRNA thus binds to the non-PAM-containing strand (the target strand). The target sequences given in the table are coding sequences of the DMD gene and thus can be either the target or non-target strand. For example, gRNA SEQ ID NO: 1 (GUAUUCAUUCAACAUUACGUCAA) binds to target sequence SEQ ID NO: 112 (ATTCAATTGACGTAATGTTGAATGAATA) on the strand given in the table (the coding strand), while gRNA SEQ ID NO: 6 binds to target sequence SEQ ID NO: 117 on the opposite strand as that given in the table (non-coding strand). Cas9 requires double-stranded DNA where one strand contains the PAM and the other contains the target sequence (i.e., the target strand). In aspects of the disclosure, one of the JHI40 gRNAs designed to target intron 40 is used with one of the JHI55 gRNAs designed to target intron 55. In exemplary aspects, JHI40-008 is used in combination with JHI55A-004.

Table 2 provided herein below provides donor sequence for replacement of exons 41-55 of the DMD gene. Table 2 provides the complete donor sequence; the JHI55A-004 target site sequence; the sequence for the intron 40 fragment containing branch point, poly-pyrimidine tract, and splice acceptor; the DMD exons 41-55 coding sequence; intron 55 fragment containing splice donor site; and the JHI40-008 target site. The complete donor sequence contains contain 1) coding sequence of the exons, 2) flanking intronic elements (a downstream splice donor and upstream splice acceptor, polypyrimidine track, and branch point sequences), and 3) Cas9 target sites on the ends.

Table 3 provided herein below provides Staphylococcus aureus and Campylobacter jejuni gRNA sequences that target human DMD introns 1 or 19, including full gRNA sequences, spacer sequences of the gRNAs, the direct repeat tracrRNA sequence, and the target sequence to which the guide RNA is designed to target. In aspects of the disclosure, one of the DSAi1 or DCJi1 gRNAs designed to target intron 1 is used with one of the DSAi19 or DSJi19 gRNAs designed to target intron 19. In exemplary aspects, DSAi1-03 is used in combination with DSAi19-004.

Table 4 provided herein below provides donor sequence for replacement of exons 2-19 of the DMD gene. Table 4 provides the complete donor sequence; the DSAi19-004 target site sequence; the sequence for the upstream intronic fragment containing branchpoint, poly-pyrimidine track, and splice acceptor; the DMD exons 2-19 coding sequence; the downstream intronic fragment containing splice donor site; and the DSAi1-03 target site. The complete donor sequence contains contain 1) coding sequence of the exons, 2) flanking intronic elements (a downstream splice donor and upstream splice acceptor, polypyrimidine track, and branch point sequences), and 3) Cas9 target sites on the ends.

Table 5 provided herein below provides exemplary Cas9 coding sequences as used in the methods of the disclosure. The provision of these sequences herein is for exemplary purposes and is not meant to limit the methods of the disclosure to these particular Cas9 sequences. As set out herein above, the methods of the disclosure are meant to be practiced with any Cas9 protein or functional fragment thereof.

TABLE 1
Staphylococcus aureus gRNAs that target human DMD introns 40 or 55.

| | | | | | | | | Human | |
|---|---|---|---|---|---|---|---|---|---|
| | | | | | | | Direct | genomic | |
| | | | gRNA | | Spacer | Direct | repeat- | target | |
| | | Full gRNA | SEQ | | SEQ | repeat- | tracrRNA | sequence | Target |
| gRNA | Cas9 | sequence | ID | Spacer | ID | tracrRNA | SEQ ID | (coding | SEQ ID |
| ID | Organism | (5′-3′) | NO: | sequence | NO: | sequence | NO: | strand) | NO: |
| JH140- | Staphy- | GUAUUCAUUCA | 1 | GUAUUCAUU | 38 | GUUUUAGUACU | 75 | ATTCAATTG | 112 |
| 001 | lococcus | ACAUUACGUCA | | CAACAUUAC | | CUGGAAACAGA | | ACGTAATGT | |
| | aureus | AGUUUUAGUA | | GUCAA | | AUCUACUAAAAC | | TGAATGAAT | |
| | Cas9 | CUCUGGAAACA | | | | AAGGCAAAAUG | | A | |
| | | GAAUCUACUAA | | | | CCGUGUUUAUC | | | |
| | | AACAAGGCAAA | | | | UCGUCAACUUG | | | |
| | | AUGCCGUGUU | | | | UUGGCGAGAUU | | | |
| | | UAUCUCGUCAA | | | | UUU | | | |
| | | CUUGUUGGCG | | | | | | | |
| | | AGAUUUUU | | | | | | | |
| JHI40- | Staphy- | GUGUUAUUCA | 2 | GUGUUAUUC | 39 | GUUUUAGUACU | 76 | GTGTTATTC | 113 |
| 002 | lococcus | AUUGACGUAAU | | AAUUGACGU | | CUGGAAACAGA | | AATTGACGT | |
| | aureus | GGUUUUAGUA | | AAUG | | AUCUACUAAAAC | | AATGTTGAA | |
| | Cas9 | CUCUGGAAACA | | | | AAGGCAAAAUG | | T | |
| | | GAAUCUACUAA | | | | CCGUGUUUAUC | | | |
| | | AACAAGGCAAA | | | | UCGUCAACUUG | | | |
| | | AUGCCGUGUU | | | | UUGGCGAGAUU | | | |
| | | UAUCUCGUCAA | | | | UUU | | | |
| | | CUUGUUGGCG | | | | | | | |
| | | AGAUUUUU | | | | | | | |
| JH140- | Staphy- | GCUGAUGAAA | 3 | GCUGAUGAA | 40 | GUUUUAGUACU | 77 | ACTCATGTT | 114 |
| 004 | lococcus | UGAAUGGGCU | | AUGAAUGGG | | CUGGAAACAGA | | AGCCCATTC | |
| | aureus | AACGUUUUAG | | CUAAC | | AUCUACUAAAAC | | ATTTCATCA | |
| | Cas9 | UACUCUGGAAA | | | | AAGGCAAAAUG | | G | |
| | | CAGAAUCUACU | | | | CCGUGUUUAUC | | | |
| | | AAAACAAGGCA | | | | UCGUCAACUUG | | | |
| | | AAAUGCCGUG | | | | UUGGCGAGAUU | | | |
| | | UUUAUCUCGU | | | | UUU | | | |
| | | CAACUUGUUG | | | | | | | |
| | | GCGAGAUUUU | | | | | | | |
| | | U | | | | | | | |
| JH140- | Staphy- | GUGUGUGAAG | 4 | GUGUGUGAA | 41 | GUUUUAGUACU | 78 | ATTCATTTC | 115 |
| 005 | lococcus | AUGCUCUGAU | | GAUGCUCUG | | CUGGAAACAGA | | ATCAGAGCA | |
| | aureus | GAAGUUUUAG | | AUGAA | | AUCUACUAAAAC | | TCTTCACAC | |
| | Cas9 | UACUCUGGAAA | | | | AAGGCAAAAUG | | A | |
| | | CAGAAUCUACU | | | | CCGUGUUUAUC | | | |
| | | AAAACAAGGCA | | | | UCGUCAACUUG | | | |
| | | AAAUGCCGUG | | | | UUGGCGAGAUU | | | |
| | | UUUAUCUCGU | | | | UUU | | | |
| | | CAACUUGUUG | | | | | | | |
| | | GCGAGAUUUU | | | | | | | |
| | | U | | | | | | | |
| JHI40- | Staphy- | GACAAUAUGCA | 5 | GACAAUAUG | 42 | GUUUUAGUACU | 79 | ACTCTCTAT | 116 |
| 006 | lococcus | AAUAAAUCUAU | | CAAAUAAAUC | | CUGGAAACAGA | | AGATTTATT | |
| | aureus | AGUUUUAGUA | | UAUA | | AUCUACUAAAAC | | TGCATATTG | |
| | Cas9 | CUCUGGAAACA | | | | AAGGCAAAAUG | | T | |
| | | GAAUCUACUAA | | | | CCGUGUUUAUC | | | |
| | | AACAAGGCAAA | | | | UCGUCAACUUG | | | |
| | | AUGCCGUGUU | | | | UUGGCGAGAUU | | | |
| | | UAUCUCGUCAA | | | | UUU | | | |
| | | CUUGUUGGCG | | | | | | | |
| | | AGAUUUUU | | | | | | | |
| JH140- | Staphy- | GUGUGGACGG | 6 | GUGUGGACG | 43 | GUUUUAGUACU | 80 | TGTGGACG | 117 |
| 008 | lococcus | UCCCUAAUAAA | | GUCCCUAAU | | CUGGAAACAGA | | GTCCCTAAT | |
| | aureus | UAGUUUUAGU | | AAAUA | | AUCUACUAAAAC | | AAATAATGA | |
| | Cas9 | ACUCUGGAAAC | | | | AAGGCAAAAUG | | GT | |
| | | AGAAUCUACUA | | | | CCGUGUUUAUC | | | |
| | | AAACAAGGCAA | | | | UCGUCAACUUG | | | |
| | | AAUGCCGUGU | | | | UUGGCGAGAUU | | | |
| | | UUAUCUCGUC | | | | UUU | | | |
| | | AACUUGUUGG | | | | | | | |
| | | CGAGAUUUUU | | | | | | | |
| JHI55 | Staphy- | GUUUCUAAGA | 7 | GUUUCUAAG | 44 | GUUUUAGUACU | 81 | ATTCCACTT | 118 |
| A-001 | lococcus | CGAGGGUGUU | | ACGAGGGUG | | CUGGAAACAGA | | AACACCCTC | |
| | aureus | AAGGUUUUAG | | UUAAG | | AUCUACUAAAAC | | GTCTTAGAA | |
| | Cas9 | UACUCUGGAAA | | | | AAGGCAAAAUG | | A | |
| | | CAGAAUCUACU | | | | CCGUGUUUAUC | | | |
| | | AAAACAAGGCA | | | | UCGUCAACUUG | | | |
| | | AAAUGCCGUG | | | | UUGGCGAGAUU | | | |
| | | UUUAUCUCGU | | | | UUU | | | |
| | | CAACUUGUUG | | | | | | | |
| | | GCGAGAUUUU | | | | | | | |
| | | U | | | | | | | |
| JHI55 | Staphy- | GACUUUGCUC | 8 | GACUUUGCU | 45 | GUUUUAGUACU | 82 | ACTTTGCTC | 119 |
| A-002 | lococcus | AGAGAAAUAAC | | CAGAGAAAU | | CUGGAAACAGA | | AGAGAAATA | |
| | aureus | UUGUUUUAGU | | AACUU | | AUCUACUAAAAC | | ACTTAGGGA | |
| | Cas9 | ACUCUGGAAAC | | | | AAGGCAAAAUG | | T | |
| | | AGAAUCUACUA | | | | CCGUGUUUAUC | | | |
| | | AAACAAGGCAA | | | | UCGUCAACUUG | | | |
| | | AAUGCCGUGU | | | | UUGGCGAGAUU | | | |
| | | UUAUCUCGUC | | | | UUU | | | |
| | | AACUUGUUGG | | | | | | | |
| | | CGAGAUUUUU | | | | | | | |
| JHI55 | Staphy- | GUGUGAAAAUA | 9 | GUGUGAAAA | 46 | GUUUUAGUACU | 83 | TGTGAAAAT | 120 |
| A-004 | lococcus | AGAAUGAGAU | | UAAGAAUGA | | CUGGAAACAGA | | AAGAATGAG | |
| | aureus | GGGUUUUAGU | | GAUGG | | AUCUACUAAAAC | | ATGGCTGAA | |
| | Cas9 | ACUCUGGAAAC | | | | AAGGCAAAAUG | | T | |
| | | AGAAUCUACUA | | | | CCGUGUUUAUC | | | |
| | | AAACAAGGCAA | | | | UCGUCAACUUG | | | |
| | | AAUGCCGUGU | | | | UUGGCGAGAUU | | | |
| | | UUAUCUCGUC | | | | UUU | | | |
| | | AACUUGUUGG | | | | | | | |
| | | CGAGAUUUUU | | | | | | | |

TABLE 2
Donor sequences for replacement of Exons 41-55.

Complete donor sequence (SEQ ID NO: 149):
ATTCAGCCATCTCATTCTTATTTTCACAgcgaggaagcggaagagcgccgcggccgcACAACAGCCTTTGAAATTTTGAGAGAAGTATTTGCTGCTTGCA
AGTCGGTTGATGTGGTTAGCTAACTGCCCTGGGCCCTGTATTGGTTTTGCTCAATAGGAAATTGATCGGGAATTGCAGAAGAAGAAAGAGGAGCTGAATG
CAGTGCGTAGGCAAGCTGAGGGCTTGTCTGAGGATGGGGCCGCAATGGCAGTGGAGCCAACTCAGATCCAGCTCAGCAAGCGCTGGCGGGAAATTGAGAGC
AAATTTGCTCAGTTTCGAAGACTCAACTTTGCACAAATTCACACTGTCCGTGAAGAAACGATGATGGTGATGACTGAAGACATGCCTTTGGAAATTTCTT
ATGTGCCTTCTACTTATTTGACTGAAATCACTCATGTCTCACAAGCCCTATTAGAAGTGGAACAACTTCTCAATGCTCCTGACCTCTGTGCTAAGGACTT
TGAAGATCTCTTTAAGCAAGAGGAGTCTCTGAAGAATATAAAAGATAGTCTACAACAAAGCTCAGGTCGGATTGACATTATTCATAGCAAGAAGACAGCA
GCATTGCAAAGTGCAACGCCTGTGGAAAGGGTGAAGCTACAGGAAGCTCTCTCCCAGCTTGATTTCCAATGGGAAAAAGTTAACAAAATGTACAAGGA
CCGACAAGGGCGATTTGACAGATCTGTTGAGAAATGGCGGCGTTTTCATTATGATATAAAGATATTTAATCAGTGGCTAACAGAAGCTGAACAGTTTCTC
AGAAAGACACAAATTCCTGAGAATTGGGAACATGCTAAATACAAATGGTATCTTAAGGAACTCCAGGATGGCATTGGGCAGCGGCAAACTGTTGTCAGA
ACATTGAATGCAACTGGGGAAGAAATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGAATATCTTGTCAGAATTTCAAAGAGATTTAAATGAATTTGTTTTATGGT
TGGAGGAAGCAGATAACATTGCTAGTATCCCACTTGAACCTGGAAAAGAGCAGCAACTAAAAGAAAAGCTTGAGCAAGTCAAGTTACTGGTGGAAGAG
TTGCCCCTGCGCCAGGGAATTCTCAAACAATTAAATGAAACTGGAGGACCCGTGCTTGTAAGTGCTCCCATAAGCCCAGAAGAGCAAGATAAACTTGA
AAATAAGCTCAAGCAGACAAATCTCCAGTGGATAAAGGTTTCCAGAGCTTTACCTGAGAAACAAGGAGAAATTGAAGCTCAAATAAAAGACCTTGGGCA
GCTTGAAAAAAAGCTTGAAGACCTTGAAGAGCAGTTAAATCATCTGCTGCTGTGGTTATCTCCTATTAGGAATCAGTTGGAAATTTATAACCAACCAAAC
CAAGAAGGACCATTTGACGTTAAGGAAACTGAAATAGCAGTTCAAGCTAAACAACCGGATGTGGAAGAGATTTTGTCTAAAGGGCAGCATTTGTACAAG
GAAAAACCAGCCACTCAGCCAGTGAAGAGGAAGTTAGAAGATCTGAGCTCTGAGTGGAAGGCGGTAAACCGTTTACTTCAAGAGCTGAGGGCAAAGC
AGCCTGACCTAGCTCCTGGACTGACCACTATTGGAGCCTCTCCTACTCAGACTGTTACTCTGGTGACACAACCTGTGGTTACTAAGGAAACTGCCATCT
CCAAACTAGAAATGCCATCTTCCTTGATGTTGGAGGTACCTGCTCTGGCAGATTTCAACCGGGCTTGGACAGAACTTACCGACTGGCTTTCTCTGCTTG
ATCAAGTTATAAAATCACAGAGGGTGATGGTGGGTGACCTTGAGGATATCAACGAGATGATCATCAAGCAGAAGGCAACAATGCAGGATTTGGAACAG
AGGCGTCCCCAGTTGGAAGAACTCATTACCGCTGCCCAAAATTTGAAAAACAAGACCAGCAATCAAGAGGCTAGAACAATCATTACGGATCGAATTGAA
AGAATTCAGAATCAGTGGGATGAAGTACAAGAACACCTTCAGAACCGGAGGCAACAGTTGAATGAAATGTTAAAGGATTCAACACAATGGCTGGAAGCT
AAGGAAGAAGCTGAGCAGGTCTTAGGACAGGCCAGAGCCAAGCTTGAGTCATGGAAGGAGGGTCCCTATACAGTAGATGCAATCCAAAAGAAAATCA
CAGAAACCAAGCAGTTGGCCAAAGACCTCCGCCAGTGGCAGACAAATGTAGATGTGGCAAATGACTTGGCCCTGAAACTTCTCCGGGATTATTCTGCA
GATGATACCAGAAAAGTCCACATGATAACAGAGAATATCAATGCCTCTTGGAGAAGCATTCATAAAAGGGTGAGTGAGCGAGAGGCTGCTTTGGAAGA
AACTCATAGATTACTGCAACAGTTCCCCCTGGACCTGGAAAAGTTTCTTGCCTGGCTTACAGAAGCTGAAACAACTGCCAATGTCCTACAGGATGCTAC
CCGTAAGGAAAGGCTCCTAGAAGACTCCAAGGGAGTAAAAGAGCTGATGAAACAATGGCAAGTAAGTCAGGCATTTCCGCTTTAGCACTCTTGTGGAT
CCAATTGAACAATTCTCAGCATTTGTACTTGTAACTGACAAGCCAGGGACAAAACAAAATAGTGcggccgcggcgcgccgacgaaagggcctcgtgatacg
cACTCATTATTTATTAGGGACCGTCCACA
JHI55A-004 target site (SEQ ID NO: 150):
ATTCAGCCATCTCATTCTTATTTTCACA
Intron 40 fragment containing branch point, poly-pyrimidine track, and splice acceptor (SEQ ID
NO: 151):
ACAACAGCCTTTGAAATTTTGAGAGAAGTATTTGCTGCTTGCAAGTCGGTTGATGTGGTTAGCTAACTGCCCTGGGCCCTGTATTGGTTTTGCTCAATA
G
DMD exons 41-55 coding sequence (SEQ ID NO: 152):
GAAATTGATCGGGAATTGCAGAAGAAGAAAGAGGAGCTGAATGCAGTGCGTAGGCAAGCTGAGGGCTTGTCTGAGGATGGGGCCGCAATGGCAGTG
GAGCCAACTCAGATCCAGCTCAGCAAGCGCTGGCGGGAAATTGAGAGCAAATTTGCTCAGTTTCGAAGACTCAACTTTGCACAAATTCACACTGTCCG
TGAAGAAACGATGATGGTGATGACTGAAGACATGCCTTTGGAAATTTCTTATGTGCCTTCTACTTATTTGACTGAAATCACTCATGTCTCACAAGCCCTA
TTAGAAGTGGAACAACTTCTCAATGCTCCTGACCTCTGTGCTAAGGACTTTGAAGATCTCTTTAAGCAAGAGGAGTCTCTGAAGAATATAAAAGATAGTC
TACAACAAAGCTCAGGTCGGATTGACATTATTCATAGCAAGAAGACAGCAGCATTGCAAAGTGCAACGCCTGTGGAAAGGGTGAAGCTACAGGAAGCT
CTCTCCCAGCTTGATTTCCAATGGGAAAAAGTTAACAAAATGTACAAGGACCGACAAGGGCGATTTGACAGATCTGTTGAGAAATGGCGGCGTTTTCAT
TATGATATAAAGATATTTAATCAGTGGCTAACAGAAGCTGAACAGTTTCTCAGAAAGACACAAATTCCTGAGAATTGGGAACATGCTAAATACAAATGGT
ATCTTAAGGAACTCCAGGATGGCATTGGGCAGCGGCAAACTGTTGTCAGAACATTGAATGCAACTGGGGAAGAAATAATTCAGCAATCCTCAAAAACAG
ATGCCAGTATTCTACAGGAAAAATTGGGAAGCCTGAATCTGCGGTGGCAGGAGGTCTGCAAACAGCTGTCAGACAGAAAAAAGAGGCTAGAAGAACAA
AAGAATATCTTGTCAGAATTTCAAAGAGATTTAAATGAATTTGTTTTATGGTTGGAGGAAGCAGATAACATTGCTAGTATCCCACTTGAACCTGGAAAAG
AGCAGCAACTAAAAGAAAAGCTTGAGCAAGTCAAGTTACTGGTGGAAGAGTTGCCCCTGCGCCAGGGAATTCTCAAACAATTAAATGAAACTGGAGGAC
CCGTGCTTGTAAGTGCTCCCATAAGCCCAGAAGAGCAAGATAAACTTGAAAATAAGCTCAAGCAGACAAATCTCCAGTGGATAAAGGTTTCCAGAGCTT
TACCTGAGAAACAAGGAGAAATTGAAGCTCAAATAAAAGACCTTGGGCAGCTTGAAAAAAAGCTTGAAGACCTTGAAGAGCAGTTAAATCATCTGCTGC
TGTGGTTATCTCCTATTAGGAATCAGTTGGAAATTTATAACCAACCAAACCAAGAAGGACCATTTGACGTTAAGGAAACTGAAATAGCAGTTCAAGCTAA
ACAACCGGATGTGGAAGAGATTTTGTCTAAAGGGCAGCATTTGTACAAGGAAAAACCAGCCACTCAGCCAGTGAAGAGGAAGTTAGAAGATCTGAGCT
CTGAGTGGAAGGCGGTAAACCGTTTACTTCAAGAGCTGAGGGCAAAGCAGCCTGACCTAGCTCCTGGACTGACCACTATTGGAGCCTCTCCTACTCAG
ACTGTTACTCTGGTGACACAACCTGTGGTTACTAAGGAAACTGCCATCTCCAAACTAGAAATGCCATCTTCCTTGATGTTGGAGGTACCTGCTCTGGCA
GATTTCAACCGGGCTTGGACAGAACTTACCGACTGGCTTTCTCTGCTTGATCAAGTTATAAAATCACAGAGGGTGATGGTGGGTGACCTTGAGGATATC
AACGAGATGATCATCAAGCAGAAGGCAACAATGCAGGATTTGGAACAGAGGCGTCCCCAGTTGGAAGAACTCATTACCGCTGCCCAAAATTTGAAAAA
CAAGACCAGCAATCAAGAGGCTAGAACAATCATTACGGATCGAATTGAAAGAATTCAGAATCAGTGGGATGAAGTACAAGAACACCTTCAGAACCGGA
GGCAACAGTTGAATGAAATGTTAAAGGATTCAACACAATGGCTGGAAGCTAAGGAAGAAGCTGAGCAGGTCTTAGGACAGGCCAGAGCCAAGCTTGAG
TCATGGAAGGAGGGTCCCTATACAGTAGATGCAATCCAAAAGAAAATCACAGAAACCAAGCAGTTGGCCAAAGACCTCCGCCAGTGGCAGACAAATGT
AGATGTGGCAAATGACTTGGCCCTGAAACTTCTCCGGGATTATTCTGCAGATGATACCAGAAAAGTCCACATGATAACAGAGAATATCAATGCCTCTTG
GAGAAGCATTCATAAAAGGGTGAGTGAGCGAGAGGCTGCTTTGGAAGAAACTCATAGATTACTGCAACAGTTCCCCCTGGACCTGGAAAAGTTTCTTG
CCTGGCTTACAGAAGCTGAAACAACTGCCAATGTCCTACAGGATGCTACCCGTAAGGAAAGGCTCCTAGAAGACTCCAAGGGAGTAAAAGAGCTGATG
AAACAATGGCAA
Intron 55 fragment containing splice donor site (SEQ ID NO: 153):
GTAAGTCAGGCATTTCCGCTTTAGCACTCTTGTGGATCCAATTGAACAATTCTCAGCATTTGTACTTGTAACTGACAAGCCAGGGACAAAACAAAATAGT
JHI40-008 target site (SEQ ID NO: 154):
ACTCATTATTTATTAGGGACCGTCCACA
Complete donor sequence 2 with introns (SEQ ID NO: 187):
ATTCAGCCATCTCATTCTTATTTTCACATCTTGCGTTTCTGATAGGCACCTATtggtCTTACTGACATCCACTTTGCCTTTCTCTcCaCAGGAAATTGA
TCGGGAATTGCAGAAGAAGAAAGAGGAGCTGAATGCAGTGCGTAGGCAAGCTGAGGGCTTGTCTGAGGATGGGGCCGCAATGGCAGTGGAGCCAACTCAG
ATCCAGCTCAGCAAGCGCTGGCGGGAAATTGAGAGCAAATTTGCTCAGTTTCGAAGACTCAACTTTGCACAAATTCACACTGTCCGTGAAGAAACGATG
ATGGTGATGACTGAAGACATGCCTTTGGAAATTTCTTATGTGCCTTCTACTTATTTGACTGAAATCACTCATGTCTCACAAGCCCTATTAGAAGTGGAAC
AACTTCTCAATGCTCCTGACCTCTGTGCTAAGGACTTTGAAGATCTCTTTAAGCAAGAGGAGTCTCTGAAGGTATAAAATCTTACCTTTTATTCAAATTAT
AAGTTTTGCGTATGTGTAAAGCCAAATAACACACCAAAACACATAAAAGCAAAGCATCGTTGGGTTGTCTAAAGCATTATGTTACTTCATCCCTGACCAA
TACAGAATATAAAAGATAGTCTACAACAAAGCTCAGGTCGGATTGACATTATTCATAGCAAGAAGACAGCAGCATTGCAAAGTGCAACGCCTGTGGAAA
GGGTGAAGCTACAGGAAGCTCTCTCCCAGCTTGATTTCCAATGGGAAAAAGTTAACAAAATGTACAAGGACCGACAAGGGCGATTTGACAGATCTGTT
GAGAAATGGCGGCGTTTTCATTATGATATAAAGATATTTAATCAGTGGCTAACAGAAGCTGAACAGTTTCTCAGAAAGACACAAATTCCTGAGAATTGGG
AACATGCTAAATACAAATGGTATCTTAAGGTATGGGGCTTTTAGAATTTGGGGAGGGGTCTCAACTTTATTTCACTTCCCTGTGCATTCTGAAAAGCCTC
ATTCTTAATGTCTGATTTTCAGGAACTCCAGGATGGCATTGGGCAGCGGCAAACTGTTGTCAGAACATTGAATGCAACTGGGGAAGAAATAATTCAGCA
ATCCTCAAAAACAGATGCCAGTATTCTACAGGAAAAATTGGGAAGCCTGAATCTGCGGTGGCAGGAGGTCTGCAAACAGCTGTCAGACAGAAAAAAGA
GGCTAGAAGAACAAAAGAATATCTTGTCAGAATTTCAAAGAGATTTAAATGAATTTGTTTTATGGTTGGAGGAAGCAGATAACATTGCTAGTATCCCACTT
GAACCTGGAAAAGAGCAGCAACTAAAAGAAAAGCTTGAGCAAGTCAAGGTAAATGTAACCAAGTATAACCAGATAGCCAGTTTCTGAATCATGGGAGTG
GGGAGTAATAAAATATTTTGCAACCTTTTACTCTTTAATAAACTTTAATTTTCACATTCTTCTAATTTTATGCTAAATGTCTTTTACAGTTACTGGTGGA
AGAGTTGCCCCTGCGCCAGGGAATTCTCAAACAATTAAATGAAACTGGAGGACCCGTGCTTGTAAGTGCTCCCATAAGCCCAGAAGAGCAAGATAAACTTG
AAAATAAGCTCAAGCAGACAAATCTCCAGTGGATAAAGGTTTCCAGAGCTTTACCTGAGAAACAAGGAGAAATTGAAGCTCAAATAAAAGACCTTGGGC
AGCTTGAAAAAAAGCTTGAAGACCTTGAAGAGCAGTTAAATCATCTGCTGCTGTGGTTATCTCCTATTAGGAATCAGTTGGAAATTTATAACCAACCAAA
CCAAGAAGGACCATTTGACGTTAAGGTGAGTTGCTCAACAATGTAAAATTTACCCTATCTGAATCTGCAGTTTATTAGTTCAGTCATGCTAACAAAACTG
TATCATTTCAGGAAACTGAAATAGCAGTTCAAGCTAAACAACCGGATGTGGAAGAGATTTTGTCTAAAGGGCAGCATTTGTACAAGGAAAAACCAGCCA
CTCAGCCAGTGAAGAGGAAGTTAGAAGATCTGAGCTCTGAGTGGAAGGCGGTAAACCGTTTACTTCAAGAGCTGAGGGCAAAGCAGCCTGACCTAGC
TCCTGGACTGACCACTATTGGAGCCTGTAAGTCAAGATTAGCTAATTATATAGGAGAGGGGTTGCTTGGTTGTGTAGGGTGAAAAAAGGCATAAAATAT
CTTGATGATTTGTAGGAATAACTATATAAATGATGTTCTTTCTTTCCTTCTAACCCTCACTCCAAACAGCTCCTACTCAGACTGTTACTCTGGTGACACAA
CCTGTGGTTACTAAGGAAACTGCCATCTCCAAACTAGAAATGCCATCTTCCTTGATGTTGGAGGTACCTGCTCTGGCAGATTTCAACCGGGCTTGGACA
GAACTTACCGACTGGCTTTCTCTGCTTGATCAAGTTATAAAATCACAGAGGGTGATGGTGGGTGACCTTGAGGATATCAACGAGATGATCATCAAGCAG
AAGGCAACAATGCAGGATTTGGAACAGAGGCGTCCCCAGTTGGAAGAACTCATTACCGCTGCCCAAAATTTGAAAAACAAGACCAGCAATCAAGAGGC
TAGAACAATCATTACGGATCGAAGTATGCTCTACTTGTCAGCCACGTTTTTGTATTTTCTCTGCAAGACTTCCTGATACACCCCTGCATTGATCAAGGGT
CATCAATGGAAACGTATTCTGACTTCATCCACTGTCCACTTCTTTCAGTTGAAAGAATTCAGAATCAGTGGGATGAAGTACAAGAACACCTTCAGAACCG
GAGGCAACAGTTGAATGAAATGTTAAAGGATTCAACACAATGGCTGGAAGCTAAGGAAGAAGCTGAGCAGGTCTTAGGACAGGCCAGAGCCAAGCTTG
AGTCATGGAAGGAGGGTCCCTATACAGTAGATGCAATCCAAAAGAAAATCACAGAAACCAAGCAGTTGGCCAAAGACCTCCGCCAGTGGCAGACAAAT
GTAGATGTGGCAAATGACTTGGCCCTGAAACTTCTCCGGGATTATTCTGCAGATGATACCAGAAAAGTCCACATGATAACAGAGAATATCAATGCCTCT
TGGAGAAGCATTCATAAAAGGTAAATAGTTTTATCAAATAGTCCACCCCAAAATCATTTTTTTTGCCTTTAGTTTTATATTTCTTCTTTAAAGTGCTTCA
ATTAATAAGTTCTTTCTTTTTTTTCTTGATAGGGTGAGTGAGCGAGAGGCTGCTTTGGAAGAAACTCATAGATTACTGCAACAGTTCCCCCTGGACCTGG
AAAAGTTTCTTGCCTGGCTTACAGAAGCTGAAACAACTGCCAATGTCCTACAGGATGCTACCCGTAAGGAAAGGCTCCTAGAAGACTCCAAGGGAGTAAAA
GAGCTGATGAAACAATGGCAAGTAAGTATCAAGGTTACAAGACAGGTTTAAggaGGCCAATAGAAACTGGGCTTGTCGAGACAGAgAAgATACTCATTAT
TTATTAGGGACCGTCCACA
DMD exons 41-55 coding sequence with introns (SEQ ID NO: 188):
TCTTGCGTTTCTGATAGGCACCTATtggtCTTACTGACATCCACTTTGCCTTTCTCTcCaCAGGAAATTGATCGGGAATTGCAGAAGAAGAAAGAGGAGCT
GAATGCAGTGCGTAGGCAAGCTGAGGGCTTGTCTGAGGATGGGGCCGCAATGGCAGTGGAGCCAACTCAGATCCAGCTCAGCAAGCGCTGGCGGGA
AATTGAGAGCAAATTTGCTCAGTTTCGAAGACTCAACTTTGCACAAATTCACACTGTCCGTGAAGAAACGATGATGGTGATGACTGAAGACATGCCTTT
GGAAATTTCTTATGTGCCTTCTACTTATTTGACTGAAATCACTCATGTCTCACAAGCCCTATTAGAAGTGGAACAACTTCTCAATGCTCCTGACCTCTGTG
CTAAGGACTTTGAAGATCTCTTTAAGCAAGAGGAGTCTCTGAAGGTATAAAATCTTACCTTTTATTCAAATTATAAGTTTTGCGTATGTGTAAAGCCAAAT
AACACACCAAAACACATAAAAGCAAAGCATCGTTGGGTTGTCTAAAGCATTATGTTACTTCATCCCTGACCAATACAGAATATAAAAGATAGTCTACAAC
AAAGCTCAGGTCGGATTGACATTATTCATAGCAAGAAGACAGCAGCATTGCAAAGTGCAACGCCTGTGGAAAGGGTGAAGCTACAGGAAGCTCTCTCC
CAGCTTGATTTCCAATGGGAAAAAGTTAACAAAATGTACAAGGACCGACAAGGGCGATTTGACAGATCTGTTGAGAAATGGCGGCGTTTTCATTATGAT
ATAAAGATATTTAATCAGTGGCTAACAGAAGCTGAACAGTTTCTCAGAAAGACACAAATTCCTGAGAATTGGGAACATGCTAAATACAAATGGTATCTTA
AGGTATGGGGCTTTTAGAATTTGGGGAGGGGTCTCAACTTTATTTCACTTCCCTGTGCATTCTGAAAAGCCTCATTCTTAATGTCTGATTTTCAGGAACT
CCAGGATGGCATTGGGCAGCGGCAAACTGTTGTCAGAACATTGAATGCAACTGGGGAAGAAATAATTCAGCAATCCTCAAAAACAGATGCCAGTATTCT
ACAGGAAAAATTGGGAAGCCTGAATCTGCGGTGGCAGGAGGTCTGCAAACAGCTGTCAGACAGAAAAAAGAGGCTAGAAGAACAAAAGAATATCTTGT
CAGAATTTCAAAGAGATTTAAATGAATTTGTTTTATGGTTGGAGGAAGCAGATAACATTGCTAGTATCCCACTTGAACCTGGAAAAGAGCAGCAACTAAA
AGAAAAGCTTGAGCAAGTCAAGGTAAATGTAACCAAGTATAACCAGATAGCCAGTTTCTGAATCATGGGAGTGGGGAGTAATAAAATATTTTGCAACCTT
TTACTCTTTAATAAACTTTAATTTTCACATTCTTCTAATTTTATGCTAAATGTCTTTTACAGTTACTGGTGGAAGAGTTGCCCCTGCGCCAGGGAATTCTC
AAACAATTAAATGAAACTGGAGGACCCGTGCTTGTAAGTGCTCCCATAAGCCCAGAAGAGCAAGATAAACTTGAAAATAAGCTCAAGCAGACAAATCTCC
AGTGGATAAAGGTTTCCAGAGCTTTACCTGAGAAACAAGGAGAAATTGAAGCTCAAATAAAAGACCTTGGGCAGCTTGAAAAAAAGCTTGAAGACCTTG
AAGAGCAGTTAAATCATCTGCTGCTGTGGTTATCTCCTATTAGGAATCAGTTGGAAATTTATAACCAACCAAACCAAGAAGGACCATTTGACGTTAAGGT
GAGTTGCTCAACAATGTAAAATTTACCCTATCTGAATCTGCAGTTTATTAGTTCAGTCATGCTAACAAAACTGTATCATTTCAGGAAACTGAAATAGCAGT
TCAAGCTAAACAACCGGATGTGGAAGAGATTTTGTCTAAAGGGCAGCATTTGTACAAGGAAAAACCAGCCACTCAGCCAGTGAAGAGGAAGTTAGAAG
ATCTGAGCTCTGAGTGGAAGGCGGTAAACCGTTTACTTCAAGAGCTGAGGGCAAAGCAGCCTGACCTAGCTCCTGGACTGACCACTATTGGAGCCTGT
AAGTCAAGATTAGCTAATTATATAGGAGAGGGGTTGCTTGGTTGTGTAGGGTGAAAAAAGGCATAAAATATCTTGATGATTTGTAGGAATAACTATATAA
ATGATGTTCTTTCTTTCCTTCTAACCCTCACTCCAAACAGCTCCTACTCAGACTGTTACTCTGGTGACACAACCTGTGGTTACTAAGGAAACTGCCATCT
CCAAACTAGAAATGCCATCTTCCTTGATGTTGGAGGTACCTGCTCTGGCAGATTTCAACCGGGCTTGGACAGAACTTACCGACTGGCTTTCTCTGCTTG
ATCAAGTTATAAAATCACAGAGGGTGATGGTGGGTGACCTTGAGGATATCAACGAGATGATCATCAAGCAGAAGGCAACAATGCAGGATTTGGAACAG
AGGCGTCCCCAGTTGGAAGAACTCATTACCGCTGCCCAAAATTTGAAAAACAAGACCAGCAATCAAGAGGCTAGAACAATCATTACGGATCGAAGTATG
CTCTACTTGTCAGCCACGTTTTTGTATTTTCTCTGCAAGACTTCCTGATACACCCCTGCATTGATCAAGGGTCATCAATGGAAACGTATTCTGACTTCATC
CACTGTCCACTTCTTTCAGTTGAAAGAATTCAGAATCAGTGGGATGAAGTACAAGAACACCTTCAGAACCGGAGGCAACAGTTGAATGAAATGTTAAAG
GATTCAACACAATGGCTGGAAGCTAAGGAAGAAGCTGAGCAGGTCTTAGGACAGGCCAGAGCCAAGCTTGAGTCATGGAAGGAGGGTCCCTATACAG
TAGATGCAATCCAAAAGAAAATCACAGAAACCAAGCAGTTGGCCAAAGACCTCCGCCAGTGGCAGACAAATGTAGATGTGGCAAATGACTTGGCCCTG
AAACTTCTCCGGGATTATTCTGCAGATGATACCAGAAAAGTCCACATGATAACAGAGAATATCAATGCCTCTTGGAGAAGCATTCATAAAAGGTAAATAG
TTTTATCAAATAGTCCACCCCAAAATCATTTTTTTTGCCTTTAGTTTTATATTTCTTCTTTAAAGTGCTTCAATTAATAAGTTCTTTCTTTTTTTTCTTG
ATAGGGTGAGTGAGCGAGAGGCTGCTTTGGAAGAAACTCATAGATTACTGCAACAGTTCCCCCTGGACCTGGAAAAGTTTCTTGCCTGGCTTACAGAAGCT
GAAACAACTGCCAATGTCCTACAGGATGCTACCCGTAAGGAAAGGCTCCTAGAAGACTCCAAGGGAGTAAAAGAGCTGATGAAACAATGGCAAGTAAGT
ATCAAGGTTACAAGACAGGTTTAAggaGGCCAATAGAAACTGGGCTTGTCGAGACAGAgAAgAT

TABLE 3
Staphylococcus aureus and Campylobacter jejuni gRNA sequences that target human DMD introns
1 or 19.

| | | | | | | | | Human | |
|---|---|---|---|---|---|---|---|---|---|
| | | | | | | | Direct | genomic | |
| | | | gRNA | | Spacer | Direct | repeat- | target | Target |
| | | Full gRNA | SEQ | | SEQ | repeat- | tracrRNA | sequence | SEQ |
| gRNA | Cas9 | sequence | ID | Spacer | ID | tracrRNA | SEQ ID | (coding | ID |
| ID | Organism | (5′-3′) | NO: | sequence | NO: | sequence | NO: | strand) | NO: |
| DSAi1- | Staphy- | gAAAAAUC | 10 | gAAAAAU | 47 | GUUAUAGU | 84 | AAAAATCATCCT | 121 |
| 01 | lococcus | AUCCUUUA | | CAUCCU | | ACUCUGGA | | TTAGAGAATACA | |
| | aureus | GAGAAUA | | UUAGAG | | AACAGAAU | | GAAT | |
| | | GUUAUAG | | AAUA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | gUAAUAUG | 11 | gUAAUAU | 48 | GUUAUAGU | 85 | TAATATGAAAAA | 122 |
| 02 | lococcus | AAAAAUCA | | GAAAAAU | | ACUCUGGA | | TCATCCTTTAGA | |
| | aureus | UCCUUUA | | CAUCCU | | AACAGAAU | | GAAT | |
| | | GUUAUAG | | UUA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | gUUAGAAC | 12 | gUUAGAA | 49 | GUUAUAGU | 86 | TTAGAACGGAAT | 123 |
| 03 | lococcus | GGAAUGU | | CGGAAU | | ACUCUGGA | | GTCCATTCCAGA | |
| | aureus | CCAUUCCA | | GUCCAU | | AACAGAAU | | GAGT | |
| | | GUUAUAG | | UCCA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | gCUAGGAU | 13 | gCUAGGA | 50 | GUUAUAGU | 87 | CTAGGATCTAGT | 124 |
| 04 | lococcus | CUAGUUU | | UCUAGU | | ACUCUGGA | | TTTCGTAAATTA | |
| | aureus | UCGUAAAU | | UUUCGU | | AACAGAAU | | GAGT | |
| | | GUUAUAG | | AAAU | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | GCUUUAA | 14 | GCUUUA | 51 | GUUAUAGU | 88 | ACTCAGTTCATT | 125 |
| 05 | lococcus | GCUUUUC | | AGCUUU | | ACUCUGGA | | GAGAAAAGCTTA | |
| | aureus | UCAAUGAA | | UCUCAA | | AACAGAAU | | AAGC | |
| | | GUUAUAG | | UGAA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | gCUUUUCU | 15 | gCUUUUC | 52 | GUUAUAGU | 89 | ATTCCATTACTC | 126 |
| 06 | lococcus | CAAUGAAC | | UCAAUG | | ACUCUGGA | | AGTTCATTGAGA | |
| | aureus | UGAGUAA | | AACUGA | | AACAGAAU | | AAAG | |
| | | GUUAUAG | | GUAA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi1- | Staphy- | gUACUUUU | 16 | gUACUUU | 53 | GUUAUAGU | 90 | ACCCCATAGAAT | 127 |
| 07 | lococcus | CUCUUACA | | UCUCUU | | ACUCUGGA | | GTGTAAGAGAA | |
| | aureus | CAUUCUA | | ACACAUU | | AACAGAAU | | AAGTA | |
| | | GUUAUAG | | CUA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DCJi1- | Campy- | gAAAAUCA | 17 | gAAAAUC | 54 | GUUAUAGU | 91 | AAAATCATCTCT | 128 |
| 01 | lobacter | UCUCUAAU | | AUCUCU | | CCCUGAAA | | AATTTGATCAAT | |
| | jejuni | UUGAUCA | | AAUUUG | | AGGGACUA | | ATGTAC | |
| | | GUUAUAG | | AUCA | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gUAAUAUG | 18 | gUAAUAU | 55 | GUUAUAGU | 92 | TAATATGAAAAA | 129 |
| 02 | lobacter | AAAAAUCA | | GAAAAAU | | CCCUGAAA | | TCATCCTTTAGA | |
| | jejuni | UCCUUUA | | CAUCCU | | AGGGACUA | | GAATAC | |
| | | GUUAUAG | | UUA | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gUACCCCA | 19 | gUACCCC | 56 | GUUAUAGU | 93 | TACCCCATAGAA | 130 |
| 03 | lobacter | UAGAAUG | | AUAGAAU | | CCCUGAAA | | TGTGTAAGAGAA | |
| | jejuni | UGUAAGA | | GUGUAA | | AGGGACUA | | AAGTAC | |
| | | GGUUAUA | | GAG | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gCUCAUUC | 20 | gCUCAUU | 57 | GUUAUAGU | 94 | CTCATTCCTGGC | 131 |
| 04 | lobacter | CUGGCAC | | CCUGGC | | CCCUGAAA | | ACTCATCTTTAT | |
| | jejuni | UCAUCUU | | ACUCAU | | AGGGACUA | | TTGCAC | |
| | | UGUUAUA | | CUUU | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gAUCUCUA | 21 | gAUCUCU | 58 | GUUAUAGU | 95 | ATCTCTAATTTG | 132 |
| 05 | lobacter | AUUUGAU | | AAUUUG | | CCCUGAAA | | ATCAATATGTAC | |
| | jejuni | CAAUAUGU | | AUCAAUA | | AGGGACUA | | TTACAC | |
| | | GUUAUAG | | UGU | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | GAUUUCC | 22 | GAUUUC | 59 | GUUAUAGU | 96 | GTGTAAGAGAA | 133 |
| 06 | lobacter | CUGUUGG | | CCUGUU | | CCCUGAAA | | AAGTACCAACA | |
| | jejuni | UACUUUU | | GGUACU | | AGGGACUA | | GGGAAATC | |
| | | CGUUAUA | | UUUC | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gUCAGCUU | 23 | gUCAGCU | 60 | GUUAUAGU | 97 | GTGTTTGCAAAA | 134 |
| 07 | lobacter | CACAGACA | | UCACAG | | CCCUGAAA | | TGCTGTCTGTGA | |
| | jejuni | GCAUUUU | | ACAGCA | | AGGGACUA | | AGCTGA | |
| | | GUUAUAG | | UUUU | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gAAACCUG | 24 | gAAACCU | 61 | GUUAUAGU | 98 | GTGCTTGGCTAT | 135 |
| 08 | lobacter | GAGGUAG | | GGAGGU | | CCCUGAAA | | GACTCTACCTCC | |
| | jejuni | AGUCAUA | | AGAGUC | | AGGGACUA | | AGGTTT | |
| | | GGUUAUA | | AUAG | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | gUUUGACA | 25 | gUUUGAC | 62 | GUUAUAGU | 99 | GTACAACCAGTT | 136 |
| 09 | lobacter | AAGUUCUA | | AAAGUU | | CCCUGAAA | | AATTAGAACTTT | |
| | jejuni | AUUAACUG | | CUAAUUA | | AGGGACUA | | GTCAAA | |
| | | UUAUAGU | | ACU | | UAAUAAAG | | | |
| | | CCCUGAAA | | | | AGUUUGCG | | | |
| | | AGGGACU | | | | GGACUCUG | | | |
| | | AUAAUAAA | | | | CGGGGUUA | | | |
| | | GAGUUUG | | | | CAAUCCCC | | | |
| | | CGGGACU | | | | UAAAACCG | | | |
| | | CUGCGGG | | | | CUUUUUUU | | | |
| | | GUUACAAU | | | | | | | |
| | | CCCCUAAA | | | | | | | |
| | | ACCGCUU | | | | | | | |
| | | UUUUU | | | | | | | |
| DCJi1- | Campy- | gCAUUUCA | 26 | gCAUUUC | 63 | GUUAUAGU | 100 | GTGTAGCCAGC | 137 |
| 10 | lobacter | AAUUCUG | | AAAUUCU | | CCCUGAAA | | CTCCGCAGAATT | |
| | jejuni | CGGAGGC | | GCGGAG | | AGGGACUA | | TGAAATG | |
| | | UGUUAUA | | GCU | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | GUUUUAC | 27 | GUUUUA | 64 | GUUAUAGU | 101 | GTATAATGAAAT | 138 |
| 11 | lobacter | ACUGAAG | | CACUGA | | CCCUGAAA | | GAGCCTTCAGT | |
| | jejuni | GCUCAUU | | AGGCUC | | AGGGACUA | | GTAAAAC | |
| | | UGUUAUA | | AUUU | | UAAUAAAG | | | |
| | | GUCCCUG | | | | AGUUUGCG | | | |
| | | AAAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DCJi1- | Campy- | GAGUACA | 28 | GAGUAC | 65 | GUUAUAGU | 102 | GTATAACGTATT | 139 |
| 12 | lobacter | GGAAAAAG | | AGGAAAA | | CCCUGAAA | | CAGCTTTTTCCT | |
| | jejuni | CUGAAUA | | AGCUGA | | AGGGACUA | | GTACTC | |
| | | GUUAUAG | | AUA | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |
| DSAi19- | Staphy- | gUUCAAGU | 29 | gUUCAAG | 66 | GUUAUAGU | 103 | TTCAAGTAATGA | 140 |
| 001 | lococcus | AAUGAUCC | | UAAUGA | | ACUCUGGA | | TCCATTTCCTCT | |
| | aureus | AUUUCCU | | UCCAUU | | AACAGAAU | | GGGT | |
| | | GUUAUAG | | UCCU | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi19- | Staphy- | gACAGACU | 30 | gACAGAC | 67 | GUUAUAGU | 104 | ACAGACTATTTC | 141 |
| 002 | lococcus | AUUUCAG | | UAUUUC | | ACUCUGGA | | AGGGGTTGTTTA | |
| | aureus | GGGUUGU | | AGGGGU | | AACAGAAU | | GAAT | |
| | | UGUUAUA | | UGUU | | CUACUAUA | | | |
| | | GUACUCU | | | | ACAAGGCA | | | |
| | | GGAAACA | | | | AAAUGCCG | | | |
| | | GAAUCUAC | | | | UGUUUAUC | | | |
| | | UAUAACAA | | | | UCGUCAAC | | | |
| | | GGCAAAAU | | | | UUGUUGGC | | | |
| | | GCCGUGU | | | | GAGAUUUU | | | |
| | | UUAUCUC | | | | UU | | | |
| | | GUCAACU | | | | | | | |
| | | UGUUGGC | | | | | | | |
| | | GAGAUUU | | | | | | | |
| | | UUU | | | | | | | |
| DSAi19- | Staphy- | gUUGUUUA | 31 | gUUGUU | 68 | GUUAUAGU | 105 | TTGTTTAGAATA | 142 |
| 003 | lococcus | GAAUAUGA | | UAGAAUA | | ACUCUGGA | | TGAGATGTGAAT | |
| | aureus | GAUGUGA | | UGAGAU | | AACAGAAU | | GGAT | |
| | | GUUAUAG | | GUGA | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi19- | Staphy- | gCUGUACA | 32 | gCUGUAC | 69 | GUUAUAGU | 106 | CTGTACACAAGT | 143 |
| 004 | lococcus | CAAGUAAU | | ACAAGUA | | ACUCUGGA | | AATAAAATTAAT | |
| | aureus | AAAAUUAG | | AUAAAAU | | AACAGAAU | | GGAT | |
| | | UUAUAGUA | | UA | | CUACUAUA | | | |
| | | CUCUGGA | | | | ACAAGGCA | | | |
| | | AACAGAAU | | | | AAAUGCCG | | | |
| | | CUACUAUA | | | | UGUUUAUC | | | |
| | | ACAAGGCA | | | | UCGUCAAC | | | |
| | | AAAUGCC | | | | UUGUUGGC | | | |
| | | GUGUUUA | | | | GAGAUUUU | | | |
| | | UCUCGUC | | | | UU | | | |
| | | AACUUGU | | | | | | | |
| | | UGGCGAG | | | | | | | |
| | | AUUUUUU | | | | | | | |
| DSAi19- | Staphy- | GGGGUUG | 33 | GGGGUU | 70 | GUUAUAGU | 107 | GGGGTTGTTTA | 144 |
| 005 | lococcus | UUUAGAAU | | GUUUAG | | ACUCUGGA | | GAATATGAGATG | |
| | aureus | AUGAGAU | | AAUAUGA | | AACAGAAU | | TGAAT | |
| | | GUUAUAG | | GAU | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DSAi19- | Staphy- | gCACAUAG | 34 | gCACAUA | 71 | GUUAUAGU | 108 | CACATAGGTTCA | 145 |
| 006 | lococcus | GUUCAUA | | GGUUCA | | ACUCUGGA | | TATTTATCAGCT | |
| | aureus | UUUAUCA | | UAUUUA | | AACAGAAU | | GAAT | |
| | | GGUUAUA | | UCAG | | CUACUAUA | | | |
| | | GUACUCU | | | | ACAAGGCA | | | |
| | | GGAAACA | | | | AAAUGCCG | | | |
| | | GAAUCUAC | | | | UGUUUAUC | | | |
| | | UAUAACAA | | | | UCGUCAAC | | | |
| | | GGCAAAAU | | | | UUGUUGGC | | | |
| | | GCCGUGU | | | | GAGAUUUU | | | |
| | | UUAUCUC | | | | UU | | | |
| | | GUCAACU | | | | | | | |
| | | UGUUGGC | | | | | | | |
| | | GAGAUUU | | | | | | | |
| | | UUU | | | | | | | |
| DSAi19- | Staphy- | gUAAACAA | 35 | gUAAACA | 72 | GUUAUAGU | 109 | ATTCACAGACTA | 146 |
| 007 | lococcus | CCCCUGA | | ACCCCU | | ACUCUGGA | | TTTCAGGGGTT | |
| | aureus | AAUAGUCU | | GAAAUA | | AACAGAAU | | GTTTA | |
| | | GUUAUAG | | GUCU | | CUACUAUA | | | |
| | | UACUCUG | | | | ACAAGGCA | | | |
| | | GAAACAGA | | | | AAAUGCCG | | | |
| | | AUCUACUA | | | | UGUUUAUC | | | |
| | | UAACAAGG | | | | UCGUCAAC | | | |
| | | CAAAAUGC | | | | UUGUUGGC | | | |
| | | CGUGUUU | | | | GAGAUUUU | | | |
| | | AUCUCGU | | | | UU | | | |
| | | CAACUUG | | | | | | | |
| | | UUGGCGA | | | | | | | |
| | | GAUUUUU | | | | | | | |
| | | U | | | | | | | |
| DCJi19- | Campy- | GUACACAA | 36 | GUACAC | 73 | GUUAUAGU | 110 | GTACACAAGTAA | 147 |
| 1 | lobacter | GUAAUAAA | | AAGUAAU | | CCCUGAAA | | TAAAATTAATGG | |
| | jejuni | AUUAAUGU | | AAAAUUA | | AGGGACUA | | ATACAC | |
| | | UAUAGUC | | AU | | UAAUAAAG | | | |
| | | CCUGAAAA | | | | AGUUUGCG | | | |
| | | GGGACUA | | | | GGACUCUG | | | |
| | | UAAUAAAG | | | | CGGGGUUA | | | |
| | | AGUUUGC | | | | CAAUCCCC | | | |
| | | GGGACUC | | | | UAAAACCG | | | |
| | | UGCGGGG | | | | CUUUUUUU | | | |
| | | UUACAAUC | | | | | | | |
| | | CCCUAAAA | | | | | | | |
| | | CCGCUUU | | | | | | | |
| | | UUUU | | | | | | | |
| DCJi19- | Campy- | gUGAAAUA | 37 | gUGAAAU | 74 | GUUAUAGU | 111 | GTGCAATATATT | 148 |
| 2 | lobacter | GUCUGUG | | AGUCUG | | CCCUGAAA | | TATTCACAGACT | |
| | jejuni | AAUAAAUA | | UGAAUAA | | AGGGACUA | | ATTTCA | |
| | | GUUAUAG | | AUA | | UAAUAAAG | | | |
| | | UCCCUGA | | | | AGUUUGCG | | | |
| | | AAAGGGA | | | | GGACUCUG | | | |
| | | CUAUAAUA | | | | CGGGGUUA | | | |
| | | AAGAGUU | | | | CAAUCCCC | | | |
| | | UGCGGGA | | | | UAAAACCG | | | |
| | | CUCUGCG | | | | CUUUUUUU | | | |
| | | GGGUUAC | | | | | | | |
| | | AAUCCCCU | | | | | | | |
| | | AAAACCGC | | | | | | | |
| | | UUUUUUU | | | | | | | |

TABLE 4
Donor sequence for replacement of Exons 2-19

Complete donor sequence (SEQ ID NO: 155):
ATCCATTAATTTTATTACTTGTGTACAGGGCCAATAGAAACTGGGCTTGTCGAGACAGAGAAGATTCTTGCGTTTCTGATAGGCACCTATTGGTCTTACT
GACATCCACTTTGCCTTTCTCTCCACAGATGAAAGAGAAGATGTTCAAAAGAAAACATTCACAAAATGGGTAAATGCACAATTTTCTAAGTTTGGGAAGC
AGCATATTGAGAACCTCTTCAGTGACCTACAGGATGGGAGGCGCCTCCTAGACCTCCTCGAAGGCCTGACAGGGCAAAAACTGCCAAAAGAAAAAGG
ATCCACAAGAGTTCATGCCCTGAACAATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAAAATATCATGGCTGGATTGCAACAAA
CCAACAGTGAAAAGATTCTCCTGAGCTGGGTCCGACAATCAACTCGTAATTATCCACAGGTTAATGTAATCAACTTCACCACCAGCTGGTCTGATGGCC
TGGCTTTGAATGCTCTCATCCATAGTCATAGGCCAGACCTATTTGACTGGAATAGTGTGGTTTGCCAGCAGTCAGCCACACAACGACTGGAACATGCAT
TCAACATCGCCAGATATCAATTAGGCATAGAGAAACTACTCGATCCTGAAGATGTTGATACCACCTATCCAGATAAGAAGTCCATCTTAATGTACATCAC
ATCACTCTTCCAAGTTTTGCCTCAACAAGTGAGCATTGAAGCCATCCAGGAAGTGGAAATGTTGCCAAGGCCACCTAAAGTGACTAAAGAAGAACATTT
TCAGTTACATCATCAAATGCACTATTCTCAACAGATCACGGTCAGTCTAGCACAGGGATATGAGAGAACTTCTTCCCCTAAGCCTCGATTCAAGAGCTAT
GCCTACACACAGGCTGCTTATGTCACCACCTCTGACCCTACACGGAGCCCATTTCCTTCACAGCATTTGGAAGCTCCTGAAGACAAGTCATTTGGCAGT
TCATTGATGGAGAGTGAAGTAAACCTGGACCGTTATCAAACAGCTTTAGAAGAAGTATTATCGTGGCTTCTTTCTGCTGAGGACACATTGCAAGCACAA
GGAGAGATTTCTAATGATGTGGAAGTGGTGAAAGACCAGTTTCATACTCATGAGGGGTACATGATGGATTTGACAGCCCATCAGGGCCGGGTTGGTAA
TATTCTACAATTGGGAAGTAAGCTGATTGGAACAGGAAAATTATCAGAAGATGAAGAAACTGAAGTACAAGAGCAGATGAATCTCCTAAATTCAAGATGG
GAATGCCTCAGGGTAGCTAGCATGGAAAAACAAAGCAATTTACATAGAGTTTTAATGGATCTCCAGAATCAGAAACTGAAAGAGTTGAATGACTGGCTA
ACAAAAACAGAAGAAAGAACAAGGAAAATGGAGGAAGAGCCTCTTGGACCTGATCTTGAAGACCTAAAACGCCAAGTACAACAACATAAGGTGCTTCAA
GAAGATCTAGAACAAGAACAAGTCAGGGTCAATTCTCTCACTCACATGGTGGTGGTAGTTGATGAATCTAGTGGAGATCACGCAACTGCTGCTTTGGAA
GAACAACTTAAGGTATTGGGAGATCGATGGGCAAACATCTGTAGATGGACAGAAGACCGCTGGGTTCTTTTACAAGACATCCTTCTCAAATGGCAACGT
CTTACTGAAGAACAGTGCCTTTTTAGTGCATGGCTTTCAGAAAAAGAAGATGCAGTGAACAAGATTCACACAACTGGCTTTAAAGATCAAAATGAAATGT
TATCAAGTCTTCAAAAACTGGCCGTTTTAAAAGCGGATCTAGAAAAGAAAAAGCAATCCATGGGCAAACTGTATTCACTCAAACAAGATCTTCTTTCAAC
ACTGAAGAATAAGTCAGTGACCCAGAAGACGGAAGCATGGCTGGATAACTTTGCCCGGTGTTGGGATAATTTAGTCCAAAAACTTGAAAAGAGTACAG
CACAGATTTCACAGGCTGTCACCACCACTCAGCCATCACTAACACAGACAACTGTAATGGAAACAGTAACTACGGTGACCACAAGGGAACAGATCCTG
GTAAAGCATGCTCAAGAGGAACTTCCACCACCACCTCCCCAAAAGAAGAGGCAGATTACTGTGGATTCTGAAATTAGGAAAAGGTTGGATGTTGATATA
ACTGAACTTCACAGCTGGATTACTCGCTCAGAAGCTGTGTTGCAGAGTCCTGAATTTGCAATCTTTCGGAAGGAAGGCAACTTCTCAGACTTAAAAGAA
AAAGTCAATGCCATAGAGCGAGAAAAAGCTGAGAAGTTCAGAAAACTGCAAGATGCCAGCAGATCAGCTCAGGCCCTGGTGGAACAGATGGTGAATG
GTAAGTATCAAGGTTACAAGACAGGTTTAAGGAACTCTCTGGAATGGACATTCCGTTCTAA
DSAi19-004 target site (SEQ ID NO: 156):
ATCCATTAATTTTATTACTTGTGTACAG
Upstream intronic fragment containing branch point, poly-pyrimidine track, and splice acceptor
sequences (SEQ ID NO: 157):
GGCCAATAGAAACTGGGCTTGTCGAGACAGAGAAGATTCTTGCGTTTCTGATAGGCACCTATTGGTCTTACTGACATCCACTTTGCCTTTCTCTCCACA
G
DMD exons 2-19 coding sequence (SEQ ID NO: 158):
ATGAAAGAGAAGATGTTCAAAAGAAAACATTCACAAAATGGGTAAATGCACAATTTTCTAAGTTTGGGAAGCAGCATATTGAGAACCTCTTCAGTGACCT
ACAGGATGGGAGGCGCCTCCTAGACCTCCTCGAAGGCCTGACAGGGCAAAAACTGCCAAAAGAAAAAGGATCCACAAGAGTTCATGCCCTGAACAAT
GTCAACAAGGCACTGCGGGTTTTGCAGAACAATAATGTTGATTTAGTGAATATTGGAAGTACTGACATCGTAGATGGAAATCATAAACTGACTCTTGGTT
TGATTTGGAATATAATCCTCCACTGGCAGGTCAAAAATGTAATGAAAAATATCATGGCTGGATTGCAACAAACCAACAGTGAAAAGATTCTCCTGAGCTG
GGTCCGACAATCAACTCGTAATTATCCACAGGTTAATGTAATCAACTTCACCACCAGCTGGTCTGATGGCCTGGCTTTGAATGCTCTCATCCATAGTCAT
AGGCCAGACCTATTTGACTGGAATAGTGTGGTTTGCCAGCAGTCAGCCACACAACGACTGGAACATGCATTCAACATCGCCAGATATCAATTAGGCATA
GAGAAACTACTCGATCCTGAAGATGTTGATACCACCTATCCAGATAAGAAGTCCATCTTAATGTACATCACATCACTCTTCCAAGTTTTGCCTCAACAAG
TGAGCATTGAAGCCATCCAGGAAGTGGAAATGTTGCCAAGGCCACCTAAAGTGACTAAAGAAGAACATTTTCAGTTACATCATCAAATGCACTATTCTCA
ACAGATCACGGTCAGTCTAGCACAGGGATATGAGAGAACTTCTTCCCCTAAGCCTCGATTCAAGAGCTATGCCTACACACAGGCTGCTTATGTCACCAC
CTCTGACCCTACACGGAGCCCATTTCCTTCACAGCATTTGGAAGCTCCTGAAGACAAGTCATTTGGCAGTTCATTGATGGAGAGTGAAGTAAACCTGGA
CCGTTATCAAACAGCTTTAGAAGAAGTATTATCGTGGCTTCTTTCTGCTGAGGACACATTGCAAGCACAAGGAGAGATTTCTAATGATGTGGAAGTGGT
GAAAGACCAGTTTCATACTCATGAGGGGTACATGATGGATTTGACAGCCCATCAGGGCCGGGTTGGTAATATTCTACAATTGGGAAGTAAGCTGATTG
GAACAGGAAAATTATCAGAAGATGAAGAAACTGAAGTACAAGAGCAGATGAATCTCCTAAATTCAAGATGGGAATGCCTCAGGGTAGCTAGCATGGAAA
AACAAAGCAATTTACATAGAGTTTTAATGGATCTCCAGAATCAGAAACTGAAAGAGTTGAATGACTGGCTAACAAAAACAGAAGAAAGAACAAGGAAAAT
GGAGGAAGAGCCTCTTGGACCTGATCTTGAAGACCTAAAACGCCAAGTACAACAACATAAGGTGCTTCAAGAAGATCTAGAACAAGAACAAGTCAGGG
TCAATTCTCTCACTCACATGGTGGTGGTAGTTGATGAATCTAGTGGAGATCACGCAACTGCTGCTTTGGAAGAACAACTTAAGGTATTGGGAGATCGAT
GGGCAAACATCTGTAGATGGACAGAAGACCGCTGGGTTCTTTTACAAGACATCCTTCTCAAATGGCAACGTCTTACTGAAGAACAGTGCCTTTTTAGTG
CATGGCTTTCAGAAAAAGAAGATGCAGTGAACAAGATTCACACAACTGGCTTTAAAGATCAAAATGAAATGTTATCAAGTCTTCAAAAACTGGCCGTTTT
AAAAGCGGATCTAGAAAAGAAAAAGCAATCCATGGGCAAACTGTATTCACTCAAACAAGATCTTCTTTCAACACTGAAGAATAAGTCAGTGACCCAGAA
GACGGAAGCATGGCTGGATAACTTTGCCCGGTGTTGGGATAATTTAGTCCAAAAACTTGAAAAGAGTACAGCACAGATTTCACAGGCTGTCACCACCA
CTCAGCCATCACTAACACAGACAACTGTAATGGAAACAGTAACTACGGTGACCACAAGGGAACAGATCCTGGTAAAGCATGCTCAAGAGGAACTTCCA
CCACCACCTCCCCAAAAGAAGAGGCAGATTACTGTGGATTCTGAAATTAGGAAAAGGTTGGATGTTGATATAACTGAACTTCACAGCTGGATTACTCGC
TCAGAAGCTGTGTTGCAGAGTCCTGAATTTGCAATCTTTCGGAAGGAAGGCAACTTCTCAGACTTAAAAGAAAAAGTCAATGCCATAGAGCGAGAAAAA
GCTGAGAAGTTCAGAAAACTGCAAGATGCCAGCAGATCAGCTCAGGCCCTGGTGGAACAGATGGTGAATG
Downstream intronic fragment containing splice donor sequence (SEQ ID NO: 159):
GTAAGTATCAAGGTTACAAGACAGGTTTAAGGA
DSAi1-03 target sequence (SEQ ID NO: 160):
ACTCTCTGGAATGGACATTCCGTTCTAA

TABLE 5
Exemplary Cas9 Coding Sequences.

| SEQ ID | | |
|---|---|---|
| NO: | Species | Cas9 Coding Sequence |
| 161 | S. aureus | atggccccaaagaagaagcggaaggtcggtatccacggagtcccagcagccaagcggaactacatcctgggcctggacatcggcatcaccagcgtgggcta |
| | | cggcatcatcgactacgagacacgggacgtgatcgatgccggcgtgcggctgttcaaagaggccaacgtggaaaacaacgagggcaggcggagcaagag |
| | | aggcgccagaaggctgaagcggcggaggcggcatagaatccagagagtgaagaagctgctgttcgactacaacctgctgaccgaccacagcgagctgagc |
| | | ggcatcaacccctacgaggccagagtgaagggcctgagccagaagctgagcgaggaagagttctctgccgccctgctgcacctggccaagagaagaggcgt |
| | | gcacaacgtgaacgaggtggaagaggacaccggcaacgagctgtccaccaaagagcagatcagccggaacagcaaggccctggaagagaaatacgtgg |
| | | ccgaactgcagctggaacggctgaagaaagacggcgaagtgcggggcagcatcaacagattcaagaccagcgactacgtgaaagaagccaaacagctgc |
| | | tgaaggtgcagaaggcctaccaccagctggaccagagcttcatcgacacctacatcgacctgctggaaacccggcggacctactatgagggacctggcgagg |
| | | gcagccccttcggctggaaggacatcaaagaatggtacgagatgctgatgggccactgcacctacttccccgaggaactgcggagcgtgaagtacgcctacaa |
| | | cgccgacctgtacaacgccctgaacgacctgaacaatctcgtgatcaccagggacgagaacgagaagctggaatattacgagaagttccagatcatcgagaa |
| | | cgtgttcaagcagaagaagaagcccaccctgaagcagatcgccaaagaaatcctcgtgaacgaagaggatattaagggctacagagtgaccagcaccggc |
| | | aagcccgagttcaccaacctgaaggtgtaccacgacatcaaggacattaccgcccggaaagagattattgagaacgccgagctgctggatcagattgccaag |
| | | atcctgaccatctaccagagcagcgaggacatccaggaagaactgaccaatctgaactccgagctgacccaggaagagatcgagcagatctctaatctgaag |
| | | ggctataccggcacccacaacctgagcctgaaggccatcaacctgatcctggacgagctgtggcacaccaacgacaaccagatcgctatcttcaaccggctga |
| | | agctggtgcccaagaaggtggacctgtcccagcagaaagagatccccaccaccctggtggacgacttcatcctgagccccgtcgtgaagagaagcttcatcca |
| | | gagcatcaaagtgatcaacgccatcatcaagaagtacggcctgcccaacgacatcattatcgagctggcccgcgagaagaactccaaggacgcccagaaaa |
| | | tgatcaacgagatgcagaagcggaaccggcagaccaacgagcggatcgaggaaatcatccggaccaccggcaaagagaacgccaagtacctgatcgag |
| | | aagatcaagctgcacgacatgcaggaaggcaagtgcctgtacagcctggaagccatccctctggaagatctgctgaacaaccccttcaactatgaggtggacc |
| | | acatcatccccagaagcgtgtccttcgacaacagcttcaacaacaaggtgctcgtgaagcaggaagaaaacagcaagaagggcaaccggaccccattccag |
| | | tacctgagcagcagcgacagcaagatcagctacgaaaccttcaagaagcacatcctgaatctggccaagggcaagggcagaatcagcaagaccaagaaa |
| | | gagtatctgctggaagaacgggacatcaacaggttctccgtgcagaaagacttcatcaaccggaacctggtggataccagatacgccaccagaggcctgatga |
| | | acctgctgcggagctacttcagagtgaacaacctggacgtgaaagtgaagtccatcaatggcggcttcaccagctttctgcggcggaagtggaagtttaagaaa |
| | | gagcggaacaaggggtacaagcaccacgccgaggacgccctgatcattgccaacgccgatttcatcttcaaagagtggaagaaactggacaaggccaaaa |
| | | aagtgatggaaaaccagatgttcgaggaaaagcaggccgagagcatgcccgagatcgaaaccgagcaggagtacaaagagatcttcatcaccccccacca |
| | | gatcaagcacattaaggacttcaaggactacaagtacagccaccgggtggacaagaagcctaatagagagctgattaacgacaccctgtactccacccggaa |
| | | ggacgacaagggcaacaccctgatcgtgaacaatctgaacggcctgtacgacaaggacaatgacaagctgaaaaagctgatcaacaagagccccgaaaa |
| | | gctgctgatgtaccaccacgacccccagacctaccagaaactgaagctgattatggaacagtacggcgacgagaagaatcccctgtacaagtactacgagga |
| | | aaccgggaactacctgaccaagtactccaaaaaggacaacggccccgtgatcaagaagattaagtattacggcaacaaactgaacgcccatctggacatcac |
| | | cgacgactaccccaacagcagaaacaaggtcgtgaagctgtccctgaagccctacagattcgacgtgtacctggacaatggcgtgtacaagttcgtgaccgtga |
| | | agaatctggatgtgatcaaaaaagaaaactactacgaagtgaatagcaagtgctatgaggaagctaagaagctgaagaagatcagcaaccaggccgagttta |
| | | tcgcctccttctacaacaacgatctgatcaagatcaacggcgagctgtatagagtgatcggcgtgaacaacgacctgctgaaccggatcgaagtgaacatgatc |
| | | gacatcacctaccgcgagtacctggaaaacatgaacgacaagaggccccccaggatcattaagacaatcgcctccaagacccagagcattaagaagtacag |
| | | cacagacattctgggcaacctgtatgaagtgaaatctaagaagcaccctcagatcatcaaaaagggcaaaaggccggcggccacgaaaaaggccggccag |
| | | gcaaaaaagaaaaagtaa |
| 162 | C. jejuni | ATGGCCCCAAAGAAGAAGCGGAAGGTCGGTGCTCGCATACTCGCTTTTGATATTGGAATTTCATCCATAGGAT |
| | | GGGCATTTTCAGAAAATGATGAACTTAAAGATTGTGGAGTCAGGATTTTCACAAAAGTAGAGAATCCCAAAACA |
| | | GGGGAAAGCCTTGCTCTCCCAAGGAGACTGGCGCGATCCGCAAGGAAACGACTTGCTAGGCGCAAAGCAAG |
| | | GTTGAATCATCTTAAACATCTCATTGCTAATGAATTTAAACTCAATTATGAAGATTACCAAAGTTTTGATGAATCT |
| | | TTGGCTAAAGCGTATAAAGGTAGTCTCATTTCCCCATATGAACTCCGTTTTCGCGCATTGAATGAACTTCTCTC |
| | | TAAACAAGATTTTGCTCGTGTCATTCTTCACATTGCAAAACGTCGCGGTTATGATGATATTAAGAATTCAGATGA |
| | | TAAGGAAAAGGGAGCGATTCTCAAAGCTATTAAACAAAATGAGGAGAAATTGGCTAACTATCAATCTGTCGGA |
| | | GAATATCTCTATAAGGAATATTTCCAAAAGTTTAAGGAAAATTCCAAGGAATTTACAAATGTGCGAAATAAGAAG |
| | | GAGTCCTATGAAAGGTGCATTGCTCAATCCTTTCTCAAAGACGAACTCAAACTCATCTTTAAGAAACAAAGGGA |
| | | ATTTGGGTTTAGTTTTAGTAAGAAGTTTGAAGAGGAAGTATTGTCAGTGGCTTTCTATAAACGGGCTCTCAAGG |
| | | ACTTTTCTCATCTGGTCGGAAATTGTTCTTTCTTTACGGATGAAAAGCGGGCACCGAAGAATTCACCACTCGCG |
| | | TTTATGTTTGTCGCACTCACTCGCATTATTAATCTCCTCAATAACCTTAAGAATACAGAAGGAATTCTTTATACAA |
| | | AAGATGATCTCAATGCGCTGCTTAATGAAGTTTTGAAGAATGGAACTCTTACTTATAAACAAACAAAGAAGTTG |
| | | CTTGGGTTGTCAGATGATTATGAATTCAAAGGAGAGAAAGGTACTTATTTTATCGAGTTTAAGAAATATAAAGAG |
| | | TTTATTAAAGCACTCGGAGAACATAATCTCTCCCAAGACGACCTTAATGAAATTGCAAAAGATATTACACTCATT |
| | | AAAGATGAAATAAAACTGAAGAAAGCACTTGCAAAATATGATCTGAATCAAAATCAAATCGATTCACTTTCTAAA |
| | | TTGGAGTTTAAAGACCATTTGAATATTTCTTTCAAAGCACTTAAATTGGTCACACCACTCATGCTTGAGGGGAA |
| | | GAAATACGATGAAGCCTGTAATGAGCTTAATTTGAAAGTCGCTATTAATGAAGATAAGAAGGATTTTCTTCCAG |
| | | CTTTTAATGAAACCTATTATAAAGATGAGGTTACGAATCCGGTTGTCTTGCGAGCAATTAAGGAATATAGGAAA |
| | | GTACTCAACGCTTTGCTCAAGAAGTATGGTAAAGTACATAAAATTAATATTGAACTTGCCCGCGAGGTCGGTAA |
| | | GAATCATTCACAACGGGCTAAAATTGAAAAGGAGCAAAATGAAAATTATAAAGCGAAGAAAGACGCAGAACTC |
| | | GAGTGTGAAAAGTTGGGCCTCAAAATTAATTCCAAGAATATACTCAAGCTTCGGCTGTTTAAGGAACAAAAGGA |
| | | GTTTTGTGCATATAGTGGAGAGAAAATCAAAATCTCCGATCTTCAAGACGAAAAGATGCTGGAAATTGACCATA |
| | | TTTATCCATATTCTAGGTCTTTTGATGATAGTTATATGAATAAAGTCCTTGTATTTACAAAACAAAACCAGGAGAA |
| | | ACTTAACCAAACTCCCTTTGAGGCTTTTGGGAATGATTCCGCAAAATGGCAAAAGATTGAAGTATTGGCTAAGA |
| | | ATCTCCCGACCAAGAAACAGAAACGAATTTTGGATAAGAACTATAAAGATAAAGAGCAGAAGAATTTTAAAGAT |
| | | AGAAATCTCAATGATACTCGATACATTGCTCGCCTTGTCTTGAATTATACCAAAGACTATTTGGACTTTCTCCCC |
| | | CTCTCAGATGATGAAAATACCAAATTGAATGACACTCAAAAGGGATCAAAAGTCCATGTTGAGGCCAAAAGTG |
| | | GGATGCTCACTTCCGCACTCCGCCATACGTGGGGATTTTCCGCAAAAGACAGGAATAATCACCTGCATCATGC |
| | | TATAGATGCTGTTATAATAGCATATGCAAATAATTCCATTGTCAAAGCCTTTTCTGATTTTAAGAAGGAACAGGA |
| | | AAGTAATTCTGCAGAATTGTATGCTAAGAAGATTTCCGAACTCGATTATAAGAATAAAAGAAAATTCTTTGAACC |
| | | ATTTAGTGGGTTTCGGCAAAAGGTCTTGGACAAAATTGATGAAATATTTGTCAGCAAACCAGAAAGGAAGAAAC |
| | | CATCCGGAGCGCTTCATGAAGAGACTTTTCGGAAGGAAGAGGAATTTTATCAAAGCTATGGCGGAAAAGAGG |
| | | GAGTTCTTAAAGCGTTGGAGCTCGGTAAAATACGGAAGGTCAATGGTAAAATAGTTAAGAACGGGGATATGTT |
| | | TAGGGTTGATATATTTAAACATAAGAAAACAAATAAATTTTATGCTGTTCCCATTTATACTATGGACTTTGCATTG |
| | | AAAGTCTTGCCGAATAAAGCGGTCGCTAGGTCCAAGAAAGGAGAGATTAAAGACTGGATATTGATGGATGAAA |
| | | ACTACGAATTTTGCTTTTCCTTGTATAAAGATAGCCTGATTTTGATACAAACCAAAGATATGCAGGAACCAGAAT |
| | | TTGTTTATTATAATGCGTTTACAAGTAGTACTGTCAGCCTTATTGTCTCCAAACATGACAATAAATTTGAAACCC |
| | | TCAGTAAGAATCAGAAAATTTTGTTTAAGAATGCGAATGAGAAAGAGGTTATTGCAAAATCCATTGGAATTCAAA |
| | | ATTTGAAGGTATTCGAGAAGTATATTGTCAGCGCGCTCGGAGAGGTTACTAAAGCTGAATTCCGCCAACGCGA |
| | | AGATTTCAAGAAAAAAAGGCCGGCGGCCACGAAAAAGGCCGGCCAGGCAAAAAAGAAAAAGTGA |
| 181 | S.aureus- | ATGGCCGATCCCAGGGGTATCTTGAAGGCATTTCCCAAGCGGCAGAAAATTCATGCTGATGCATCATCAAAAG |
| | Cas9-hPoIL- | TACTTGCAAAGATTCCTAGGAGGGAAGAGGGAGAAGAAAAGCGGAACTACATCctgggcctggacatcggcatcaccagc |
| | NLS | gtgggctacggcatcatcgactacgagacacgggacgtgatcgatgccggcgtgcggctgttcaaagaggccaacgtggaaaacaacgagggcaggcgga |
| | | gcaagagaggcgccagaaggctgaagcggcggaggcggcatagaatccagagagtgaagaagctgctgttcgactacaacctgctgaccgaccacagcg |
| | | agctgagcggcatcaacccctacgaggccagagtgaagggcctgagccagaagctgagcgaggaagagttctctgccgccctgctgcacctggccaagaga |
| | | agaggcgtgcacaacgtgaacgaggtggaagaggacaccggcaacgagctgtccaccaaagagcagatcagccggaacagcaaggccctggaagaga |
| | | aatacgtggccgaactgcagctggaacggctgaagaaagacggcgaagtgcggggcagcatcaacagattcaagaccagcgactacgtgaaagaagcca |
| | | aacagctgctgaaggtgcagaaggcctaccaccagctggaccagagcttcatcgacacctacatcgacctgctggaaacccggcggacctactatgagggac |
| | | ctggcgagggcagccccttcggctggaaggacatcaaagaatggtacgagatgctgatgggccactgcacctacttccccgaggaactgcggagcgtgaagt |
| | | acgcctacaacgccgacctgtacaacgccctgaacgacctgaacaatctcgtgatcaccagggacgagaacgagaagctggaatattacgagaagttccaga |
| | | tcatcgagaacgtgttcaagcagaagaagaagcccaccctgaagcagatcgccaaagaaatcctcgtgaacgaagaggatattaagggctacagagtgacc |
| | | agcaccggcaagcccgagttcaccaacctgaaggtgtaccacgacatcaaggacattaccgcccggaaagagattattgagaacgccgagctgctggatcag |
| | | attgccaagatcctgaccatctaccagagcagcgaggacatccaggaagaactgaccaatctgaactccgagctgacccaggaagagatcgagcagatctct |
| | | aatctgaagggctataccggcacccacaacctgagcctgaaggccatcaacctgatcctggacgagctgtggcacaccaacgacaaccagatcgctatcttca |
| | | accggctgaagctggtgcccaagaaggtggacctgtcccagcagaaagagatccccaccaccctggtggacgacttcatcctgagccccgtcgtgaagagaa |
| | | gottcatccagagcatcaaagtgatcaacgccatcatcaagaagtacggcctgcccaacgacatcattatcgagctggcccgcgagaagaactccaaggacg |
| | | cccagaaaatgatcaacgagatgcagaagcggaaccggcagaccaacgagcggatcgaggaaatcatccggaccaccggcaaagagaacgccaagta |
| | | cctgatcgagaagatcaagctgcacgacatgcaggaaggcaagtgcctgtacagcctggaagccatccctctggaagatctgctgaacaaccccttcaactatg |
| | | aggtggaccacatcatccccagaagcgtgtccttcgacaacagcttcaacaacaaggtgctcgtgaagcaggaagaaaacagcaagaagggcaaccggac |
| | | cccattccagtacctgagcagcagcgacagcaagatcagctacgaaaccttcaagaagcacatcctgaatctggccaagggcaagggcagaatcagcaaga |
| | | ccaagaaagagtatctgctggaagaacgggacatcaacaggttctccgtgcagaaagacttcatcaaccggaacctggtggataccagatacgccaccagag |
| | | gcctgatgaacctgctgcggagctacttcagagtgaacaacctggacgtgaaagtgaagtccatcaatggcggcttcaccagctttctgcggcggaagtggaagt |
| | | ttaagaaagagcggaacaaggggtacaagcaccacgccgaggacgccctgatcattgccaacgccgatttcatcttcaaagagtggaagaaactggacaag |
| | | gccaaaaaagtgatggaaaaccagatgttcgaggaaaagcaggccgagagcatgcccgagatcgaaaccgagcaggagtacaaagagatcttcatcacc |
| | | ccccaccagatcaagcacattaaggacttcaaggactacaagtacagccaccgggtggacaagaagcctaatagagagctgattaacgacaccctgtactcc |
| | | acccggaaggacgacaagggcaacaccctgatcgtgaacaatctgaacggcctgtacgacaaggacaatgacaagctgaaaaagctgatcaacaagagc |
| | | cccgaaaagctgctgatgtaccaccacgacccccagacctaccagaaactgaagctgattatggaacagtacggcgacgagaagaatcccctgtacaagtac |
| | | tacgaggaaaccgggaactacctgaccaagtactccaaaaaggacaacggccccgtgatcaagaagattaagtattacggcaacaaactgaacgcccatct |
| | | ggacatcaccgacgactaccccaacagcagaaacaaggtcgtgaagctgtccctgaagccctacagattcgacgtgtacctggacaatggcgtgtacaagttc |
| | | gtgaccgtgaagaatctggatgtgatcaaaaaagaaaactactacgaagtgaatagcaagtgctatgaggaagctaagaagctgaagaagatcagcaacca |
| | | ggccgagtttatcgcctccttctacaacaacgatctgatcaagatcaacggcgagctgtatagagtgatcggcgtgaacaacgacctgctgaaccggatcgaagt |
| | | gaacatgatcgacatcacctaccgcgagtacctggaaaacatgaacgacaagaggccccccaggatcattaagacaatcgcctccaagacccagagcatta |
| | | agaagtacagcacagacattctgggcaacctgtatgaagtgaaatctaagaagcaccctcagatcatcaaaaagggcaaaaggccggcggccacgaaaaa |
| | | ggccggccaggcaaaaaagaaaaagTAA |
| 183 | C. jejuni- | ATGGCCGATCCCAGGGGTATCTTGAAGGCATTTCCCAAGCGGCAGAAAATTCATGCTGATGCATCATCAAAAG |
| | Cas9-hPoIL- | TACTTGCAAAGATTCCTAGGAGGGAAGAGGGAGAAGAAGCTCGCATACTCGCTTTTGATATTGGAATTTCATC |
| | NLS | CATAGGATGGGCATTTTCAGAAAATGATGAACTTAAAGATTGTGGAGTCAGGATTTTCACAAAAGTAGAGAATC |
| | | CCAAAACAGGGGAAAGCCTTGCTCTCCCAAGGAGACTGGCGCGATCCGCAAGGAAACGACTTGCTAGGCGC |
| | | AAAGCAAGGTTGAATCATCTTAAACATCTCATTGCTAATGAATTTAAACTCAATTATGAAGATTACCAAAGTTTT |
| | | GATGAATCTTTGGCTAAAGCGTATAAAGGTAGTCTCATTTCCCCATATGAACTCCGTTTTCGCGCATTGAATGA |
| | | ACTTCTCTCTAAACAAGATTTTGCTCGTGTCATTCTTCACATTGCAAAACGTCGCGGTTATGATGATATTAAGAA |
| | | TTCAGATGATAAGGAAAAGGGAGCGATTCTCAAAGCTATTAAACAAAATGAGGAGAAATTGGCTAACTATCAAT |
| | | CTGTCGGAGAATATCTCTATAAGGAATATTTCCAAAAGTTTAAGGAAAATTCCAAGGAATTTACAAATGTGCGAA |
| | | ATAAGAAGGAGTCCTATGAAAGGTGCATTGCTCAATCCTTTCTCAAAGACGAACTCAAACTCATCTTTAAGAAA |
| | | CAAAGGGAATTTGGGTTTAGTTTTAGTAAGAAGTTTGAAGAGGAAGTATTGTCAGTGGCTTTCTATAAACGGGC |
| | | TCTCAAGGACTTTTCTCATCTGGTCGGAAATTGTTCTTTCTTTACGGATGAAAAGCGGGCACCGAAGAATTCAC |
| | | CACTCGCGTTTATGTTTGTCGCACTCACTCGCATTATTAATCTCCTCAATAACCTTAAGAATACAGAAGGAATTC |
| | | TTTATACAAAAGATGATCTCAATGCGCTGCTTAATGAAGTTTTGAAGAATGGAACTCTTACTTATAAACAAACAA |
| | | AGAAGTTGCTTGGGTTGTCAGATGATTATGAATTCAAAGGAGAGAAAGGTACTTATTTTATCGAGTTTAAGAAA |
| | | TATAAAGAGTTTATTAAAGCACTCGGAGAACATAATCTCTCCCAAGACGACCTTAATGAAATTGCAAAAGATATT |
| | | ACACTCATTAAAGATGAAATAAAACTGAAGAAAGCACTTGCAAAATATGATCTGAATCAAAATCAAATCGATTCA |
| | | CTTTCTAAATTGGAGTTTAAAGACCATTTGAATATTTCTTTCAAAGCACTTAAATTGGTCACACCACTCATGCTT |
| | | GAGGGGAAGAAATACGATGAAGCCTGTAATGAGCTTAATTTGAAAGTCGCTATTAATGAAGATAAGAAGGATTT |
| | | TCTTCCAGCTTTTAATGAAACCTATTATAAAGATGAGGTTACGAATCCGGTTGTCTTGCGAGCAATTAAGGAAT |
| | | ATAGGAAAGTACTCAACGCTTTGCTCAAGAAGTATGGTAAAGTACATAAAATTAATATTGAACTTGCCCGCGAG |
| | | GTCGGTAAGAATCATTCACAACGGGCTAAAATTGAAAAGGAGCAAAATGAAAATTATAAAGCGAAGAAAGACG |
| | | CAGAACTCGAGTGTGAAAAGTTGGGCCTCAAAATTAATTCCAAGAATATACTCAAGCTTCGGCTGTTTAAGGAA |
| | | CAAAAGGAGTTTTGTGCATATAGTGGAGAGAAAATCAAAATCTCCGATCTTCAAGACGAAAAGATGCTGGAAAT |
| | | TGACCATATTTATCCATATTCTAGGTCTTTTGATGATAGTTATATGAATAAAGTCCTTGTATTTACAAAACAAAAC |
| | | CAGGAGAAACTTAACCAAACTCCCTTTGAGGCTTTTGGGAATGATTCCGCAAAATGGCAAAAGATTGAAGTATT |
| | | GGCTAAGAATCTCCCGACCAAGAAACAGAAACGAATTTTGGATAAGAACTATAAAGATAAAGAGCAGAAGAATT |
| | | TTAAAGATAGAAATCTCAATGATACTCGATACATTGCTCGCCTTGTCTTGAATTATACCAAAGACTATTTGGACT |
| | | TTCTCCCCCTCTCAGATGATGAAAATACCAAATTGAATGACACTCAAAAGGGATCAAAAGTCCATGTTGAGGCC |
| | | AAAAGTGGGATGCTCACTTCCGCACTCCGCCATACGTGGGGATTTTCCGCAAAAGACAGGAATAATCACCTGC |
| | | ATCATGCTATAGATGCTGTTATAATAGCATATGCAAATAATTCCATTGTCAAAGCCTTTTCTGATTTTAAGAAGG |
| | | AACAGGAAAGTAATTCTGCAGAATTGTATGCTAAGAAGATTTCCGAACTCGATTATAAGAATAAAAGAAAATTCT |
| | | TTGAACCATTTAGTGGGTTTCGGCAAAAGGTCTTGGACAAAATTGATGAAATATTTGTCAGCAAACCAGAAAGG |
| | | AAGAAACCATCCGGAGCGCTTCATGAAGAGACTTTTCGGAAGGAAGAGGAATTTTATCAAAGCTATGGCGGAA |
| | | AAGAGGGAGTTCTTAAAGCGTTGGAGCTCGGTAAAATACGGAAGGTCAATGGTAAAATAGTTAAGAACGGGGA |
| | | TATGTTTAGGGTTGATATATTTAAACATAAGAAAACAAATAAATTTTATGCTGTTCCCATTTATACTATGGACTTT |
| | | GCATTGAAAGTCTTGCCGAATAAAGCGGTCGCTAGGTCCAAGAAAGGAGAGATTAAAGACTGGATATTGATGG |
| | | ATGAAAACTACGAATTTTGCTTTTCCTTGTATAAAGATAGCCTGATTTTGATACAAACCAAAGATATGCAGGAAC |
| | | CAGAATTTGTTTATTATAATGCGTTTACAAGTAGTACTGTCAGCCTTATTGTCTCCAAACATGACAATAAATTTG |
| | | AAACCCTCAGTAAGAATCAGAAAATTTTGTTTAAGAATGCGAATGAGAAAGAGGTTATTGCAAAATCCATTGGA |
| | | ATTCAAAATTTGAAGGTATTCGAGAAGTATATTGTCAGCGCGCTCGGAGAGGTTACTAAAGCTGAATTCCGCCA |
| | | ACGCGAAGATTTCAAGAAAAAAAGGCCGGCGGCCACGAAAAAGGCCGGCCAGGCAAAAAAGAAAAAGTGA |

As set out herein above, the disclosure utilizes Homology-Independent Targeted-Integration (HITI) to accomplish high efficiency knock in using the non-homologous end-joining (NHEJ) DNA repair pathway. Thus, in some aspects, the disclosure utilizes and provides guide RNAs to target sites at a particular genomic region so that Cas9 nuclease can create double-stranded breaks. In some aspects, the disclosure includes Staphylococcus aureus gRNAs that target human DMD introns 40 or 55. In some aspects, the disclosure includes Campylobacter jejuni gRNAs that target human DMD introns 40 or 55. In some aspects, the disclosure includes Streptococcus pyogenes gRNAs that target human DMD introns 40 or 55. In some aspects, the disclosure includes Staphylococcus aureus gRNAs that target human DMD introns 1 or 19. In some aspects, the disclosure includes Campylobacter jejuni gRNAs that target human DMD introns 1 or 19. In some aspects, the disclosure includes Streptococcus pyogenes gRNAs that target human DMD introns 1 or 19.

In some aspects, the disclosure provides guide RNAs targeting DMD introns 40 or 55, wherein the nucleic acid encoding the gRNA comprises any of SEQ ID NOs: 1-9, or a variant thereof comprising at least about 70%, about 75%, about 80%, about 85%, about 90%, 91%, 92%, 93%, 94%, 95%, 96%, 97%, 98%, or 99% identity to the sequence set out in any of SEQ ID NOs: 1-9. See Table 1.

In some exemplary aspects, the disclosure provides guide RNAs targeting DMD introns 1 or 19, wherein the nucleic acid encoding the gRNA comprises any of SEQ ID NOs: 1-9, or a variant thereof comprising at least about 70%, about 75%, about 80%, about 85%, about 90%, 91%, 92%, 93%, 94%, 95%, 96%, 97%, 98%, or 99% identity to the sequence set out in any of SEQ ID NOs: 10-37. See Table 1.

In some aspects, the disclosure provides the complete donor sequence for replacement of exons 2-19 comprising the nucleotide sequence set out in SEQ ID NO: 155 or a variant thereof comprising at least about 70%, about 75%, about 80%, about 85%, about 90%, 91%, 92%, 93%, 94%, 95%, 96%, 97%, 98%, or 99% identity to SEQ ID NO: 155. In some aspects, the disclosure provides the DMD exons 2-19 coding sequence comprising the nucleotide sequence set out in SEQ ID NO: 158 or a variant thereof comprising at least about 70%, about 75%, about 80%, about 85%, about 90%, 91%, 92%, 93%, 94%, 95%, 96%, 97%, 98%, or 99% identity to SEQ ID NO: 158. See Table 4.

In some aspects, the disclosure provides human genomic target sequence for DMD introns 40 or 55, wherein the nucleic acid encoding the gRNA is designed to target. In some aspects, such DMD target sequence comprises the nucleotide sequence set out in any of SEQ ID NOs: 112-120. See Table 3.

In some aspects, the disclosure provides human genomic target sequence for DMD introns 1 or 19, wherein the nucleic acid encoding the gRNA is designed to target. In some aspects, such DMD target sequence comprises the nucleotide sequence set out in any of SEQ ID NOs: 121-148. See Table 3.

In some aspects, the disclosure provides the complete donor sequence for replacement of exons 41-55 comprising the nucleotide sequence set out in SEQ ID NO: 149 or 187 or a variant thereof comprising at least about 70%, about 75%, about 80%, about 85%, about 90%, 91%, 92%, 93%, 94%, 95%, 96%, 97%, 98%, or 99% identity to SEQ ID NO: 149 or 187. In some aspects, the disclosure provides the DMD exons 41-55 coding sequence comprising the nucleotide sequence set out in SEQ ID NO: 152 or 188, or a variant thereof comprising at least about 70%, about 75%, about 80%, about 85%, about 90%, 91%, 92%, 93%, 94%, 95%, 96%, 97%, 98%, or 99% identity to SEQ ID NO: 152 or 188. See Tables 2 and 10.

In some aspects, the disclosure provides the complete donor sequence for replacement of exons 1-19 comprising the nucleotide sequence set out in SEQ ID NO: 172 or 176, or a variant thereof comprising at least about 70%, about 75%, about 80%, about 85%, about 90%, 91%, 92%, 93%, 94%, 95%, 96%, 97%, 98%, or 99% identity to SEQ ID NO: 172 or 176. See Table 8.

In some aspects, the disclosure provides unique sequences for the various subparts of the donor sequence for replacement of exons 1-19, such sequences comprising the nucleotide sequence set out in any one of SEQ ID NOs: 173-175, 177, and 178, or a variant thereof comprising at least about 70%, about 75%, about 80%, about 85%, about 90%, 91%, 92%, 93%, 94%, 95%, 96%, 97%, 98%, or 99% identity to SEQ ID NOs: 173-175, 177, and 178. See Table 8.

The disclosure provides a nucleic acid encoding a CRISPR-associated (Cas) enzyme comprising at its 5′ end a polynucleotide encoding a nuclear localization signal comprising a nucleotide sequence comprising a nucleotide sequence comprising the nucleotide sequence set out in SEQ ID NO: 179 or a variant thereof comprising at least or about 70% identity to the nucleotide sequence set out in SEQ ID NO: 179; or a nucleotide sequence encoding the amino acid sequence set out in SEQ ID NO: 180 or a variant thereof comprising at least or about 70% identity to amino acid sequence set out in SEQ ID NO: 180. In some aspects, the Cas enzyme is Cas9 or Cas13.

In some aspects, the disclosure provides Cas9 coding sequences. In some aspects, Cas9 is mammalian codon optimized. In some aspects, Cas9 is modified with a nuclear localization sequence. In some aspects, the disclosure provides any Cas sequence modified with a nuclear localization signal.

In exemplary aspects, Cas9 is encoded by the nucleic acid comprising the nucleotide sequence set out in SEQ ID NO: 161, 162, 181 or 183 (see Table 5), a variant thereof comprising at least about 70%, about 75%, about 80%, about 85%, about 90%, 91%, 92%, 93%, 94%, 95%, 96%, 97%, 98%, or 99% identity to the sequence set out in SEQ ID NO: 161, 162, 181 or 183, or a functional fragment thereof. In some aspects, the methods of the disclosure comprise an S. aureus Cas9, such as those comprising the nucleotide sequence set out in SEQ ID NO: 161 or 181, a variant thereof comprising at least about 70%, about 75%, about 80%, about 85%, about 90%, 91%, 92%, 93%, 94%, 95%, 96%, 97%, 98%, or 99% identity to the sequence set out in SEQ ID NO: 161 or 181, or a functional fragment thereof. In some aspects, the methods of the disclosure comprise a C. jejuni Cas9, such as those comprising the nucleotide sequence set out in SEQ ID NO: 162 or 183, a variant thereof comprising at least about 70%, about 75%, about 80%, about 85%, about 90%, 91%, 92%, 93%, 94%, 95%, 96%, 97%, 98%, or 99% identity to the sequence set out in SEQ ID NO: 162 or 183, or a functional fragment thereof.

As set out herein above, the disclosure utilizes Homology-Independent Targeted-Integration (HITI) to accomplish high efficiency knock in using the non-homologous end-joining (NHEJ) DNA repair pathway to knock in a donor sequence. Thus, in some aspects, the disclosure utilizes and provides guide RNAs to target sites at a particular genomic region so that Cas9 nuclease can create double-stranded breaks for the insertion of the donor sequence. In some aspects, the donor sequence is designed to replace exons 41-55. In some aspects, the donor sequence is designed to replace exons 41-55 comprises the nucleotide sequence set forth in SEQ ID NO: 149 or 152, or 187 or 188, or a variant of any thereof comprising at least about 70%, about 75%, about 80%, about 85%, about 90%, 91%, 92%, 93%, 94%, 95%, 96%, 97%, 98%, or 99% identity to the sequence set out in SEQ ID NO: 149 or 152, or 187 or 188. In some aspects, the donor sequence is designed to replace exons 2-19. In exemplary aspects, the donor sequence designed to replace exons 2-19 comprises the nucleotide sequence set forth in SEQ ID NO: 155 or 158, or a variant thereof comprising at least about 70%, about 75%, about 80%, about 85%, about 90%, 91%, 92%, 93%, 94%, 95%, 96%, 97%, 98%, or 99% identity to the sequence set out in SEQ ID NO: 155 or 158.

In some aspects, the nucleic acid encoding Cas9 is inserted into a mammalian expression vector, including a viral vector for expression in cells. In some aspects, the nucleic acid encoding mammalian gRNA for Cas9 is cloned into a mammalian expression vector, including a viral vector for expression in cells.

In some aspects, the DNA encoding the guide RNA and/or the Cas9 are under expression of a promoter. In some aspects, the promoter is a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

In some aspects, the promoter is a U6 promoter. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002)] because (1) the promoter is recognized by RNA polymerase Ill (poly Ill) and controls high-level, constitutive expression of shRNA; and (2) the promoter is active in most mammalian cell types. In some aspects, the promoter is a type III Pol III promoter in that all elements required to control expression of the shRNA are located upstream of the transcription start site (Paule et al., Nucleic Acids Res. 28(6):1283-98 (2000)). The disclosure includes both murine and human U6 promoters. The shRNA containing the sense and antisense sequences from a target gene connected by a loop is transported from the nucleus into the cytoplasm where Dicer processes it into small/short interfering RNAs (siRNAs).

Embodiments of the disclosure utilize vectors (for example, viral vectors, such as adeno-associated virus (AAV), adenovirus, retrovirus, lentivirus, equine-associated virus, alphavirus, pox viruses, herpes virus, polio virus, sindbis virus and vaccinia viruses) to deliver polynucleotides encoding DMD RNA (donor sequence), DMD gRNAs, and Cas9 enzymes disclosed herein. In some aspects, a set of DMD gRNA and DMD donor sequence are cloned into a vector. In some aspects, a set of DMD gRNA, DMD donor sequence, and Cas9 sequence are cloned into a vector. In some aspects, each of DMD gRNA, DMD donor sequence, and Cas9 sequence are cloned each individually into its own vector. Thus, in some aspects the disclosure includes vectors comprising one or more of the nucleotide sequences described herein above in the disclosure. In some aspects, the vectors are AAV vectors. In some aspects, the vectors are single stranded AAV vectors. In some aspects the AAV is recombinant AAV (rAAV). In some aspects, the rAAV lack rep and cap genes. In some aspects, rAAV are self-complementary (sc)AAV.

In some aspects, the disclosure utilizes adeno-associated virus (AAV) to deliver nucleic acids encoding the gRNA, nucleic acids encoding donor DMD sequence, and/or nucleic acids encoding Cas9, or its orthologs or variants. AAV is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45: 555-564 {1983}; the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78: 6381-6388 (2004); the AAV10 genome is provided in Mol. Ther., 13(1): 67-76 (2006); and the AAV11 genome is provided in Virology, 330(2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may be lyophilized and AAV-infected cells are not resistant to superinfection.

Recombinant AAV genomes of the disclosure comprise one or more AAV ITRs flanking at least one DMD-targeted polynucleotide construct. In some embodiments, the polynucleotide is a gRNA or a polynucleotide encoding the gRNA. In some aspects, the gRNA is administered with other polynucleotide constructs targeting DMD. Thus, in some aspects, the polynucleotide encoding the DMD gRNA is administered with a polynucleotide encoding the DMD donor sequence. In various aspects, the gRNA is expressed under various promoters including, but not limited to, such promoters as a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter Specifically, this strategy is used, in various aspects, to achieve more efficient expression of the same gRNA in multiple copies in a single backbone. AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, and AAVrh.74. As set out herein above, the nucleotide sequences of the genomes of various AAV serotypes are known in the art.

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVanc80, and AAVrh.74. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, and AAVrh.74. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

Recombinant AAV genomes of the disclosure comprise one or more AAV ITRs flanking a polynucleotide encoding, for example, one or more guide RNAs, donor DNA sequences, or Cas9. Embodiments include a rAAV genome comprising a nucleic acid comprising a nucleotide sequence set out in any of SEQ ID NOs: 1-39.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure includes a composition comprising rAAV comprising any of the constructs described herein. In some aspects, the disclosure includes a composition comprising the rAAV for delivering the gRNA described herein. In some aspects, the disclosure includes a composition the rAAV comprising one or more of the polynucleotide sequences encoding the gRNA described herein along with one or more polynucleotide sequences encoding DMD donor sequence and/or polynucleotide sequences encoding Cas9. Compositions of the disclosure comprise rAAV and one or more pharmaceutically or physiologically acceptable carriers, excipients or diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

In some aspects, the disclosure includes a dual-plasmid system comprising one plasmid comprising the knock-in donor sequence flanked on each side of the donor sequences by a genomic Cas9 cut site and two gRNAs; and a second plasmid comprising Cas9 enzyme or a functional fragment thereof capable of generating double-stranded DNA breaks at DNA loci determined by a gRNA spacer sequence. In some aspects, the plasmids are introduced into a rAAV for delivery. In some aspects, the plasmids are introduced into the cell via non-vectorized delivery.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×10¹⁰ vg, 1×10¹¹ vg, 1×10¹² vg, 1×10¹³ vg, and 1×10¹⁴ vg, respectively).

In an embodiment, the disclosure includes non-vectorized delivery of the nucleic acids encoding the gRNAs, nucleic acids encoding donor DMD sequence, and/or nucleic acids encoding Cas9 or the functional fragment thereof. In some aspects, in this context, synthetic carriers able to form complexes with nucleic acids, and protect them from extra- and intracellular nucleases, are an alternative to viral vectors. The disclosure includes such non-vectorized delivery. The disclosure also includes compositions comprising any of the constructs described herein alone or in combination.

In some aspects, the disclosure provides a method of delivering any one or more nucleic acids comprising (i) a polynucleotide encoding the DMD gRNA comprising the nucleotide sequence set forth in any one of SEQ ID NOs: 1-6, or a variant thereof comprising at least or about 70% identity to the nucleotide sequence set forth in any one of SEQ ID NOs: 1-6; or a polynucleotide encoding a DMD gRNA targeting the nucleotide sequence set forth in any one of SEQ ID NOs: 112-117 (ii) a polynucleotide encoding the DMD gRNA comprising the nucleotide sequence set forth in any one of SEQ ID NOs: 7-9, or a variant thereof comprising at least or about 70% identity to the nucleotide sequence set forth in any one of SEQ ID NOs: 7-9; or a polynucleotide encoding a DMD gRNA targeting the nucleotide sequence set forth in any one of SEQ ID NOs: 118-120 (iii) a donor sequence for replacement of exons 41-55 comprising a polynucleotide comprising the nucleotide sequence set forth in SEQ ID NO: 149 or 152, or 187 or 188, or a variant of any thereof comprising at least about 70% identity to the nucleotide sequence set forth in in SEQ ID NO: 149 or 152, or 187 or 188; and (iv) a nucleic acid encoding a Cas9 enzyme or a functional fragment thereof to a cell or to a subject in need thereof. In some aspects, the method comprises administering to the subject an AAV comprising nucleic acids encoding (i) the DMD gRNAs (one gRNA targeting each of introns 40 and 55), (ii) the DMD donor sequence, (iii) the Cas9 enzyme or a functional fragment thereof. In some aspects, the nucleic acid encoding the Cas9 enzyme comprises the nucleotide sequence set forth in SEQ ID NO: 161, 162, 181, or 183, a variant thereof comprising at least about 70% identity to the nucleotide sequence set forth in SEQ ID NO: 161, 162, 181, or 183, or a functional fragment thereof. In some aspects, the method comprises administering to the subject the nucleic acids encoding (i) at least two DMD gRNAs, wherein at least one gRNA targets intron 40 and the other gRNA targets intron 55), (ii) the DMD donor sequence, and (iii) the Cas9 enzyme or a functional fragment thereof. In some aspects, the method comprises delivering the nucleic acids in one or more AAV vectors. In some aspects, the method comprises delivering the nucleic acids in non-vectorized delivery.

In some aspects, the disclosure provides a method of delivering any one or more nucleic acids comprising (i) a polynucleotide encoding the DMD gRNA comprising the nucleotide sequence set forth in any one of SEQ ID NOs: 10-28, or a variant thereof comprising at least or about 70% identity to the nucleotide sequence set forth in any one of SEQ ID NOs: 10-28; or a polynucleotide encoding a DMD gRNA targeting the nucleotide sequence set forth in any one of SEQ ID NOs: 121-139 (ii) a polynucleotide encoding the DMD gRNA comprising the nucleotide sequence set forth in any one of SEQ ID NOs: 29-37, or a variant thereof comprising at least or about 70% identity to the nucleotide sequence set forth in any one of SEQ ID NOs: 29-37; or a polynucleotide encoding a DMD gRNA targeting the nucleotide sequence set forth in any one of SEQ ID NOs: 140-148 (iii) a donor sequence for replacement of exons 2-19 comprising a polynucleotide comprising the nucleotide sequence set forth in SEQ ID NO: 155 or 158, or a variant thereof comprising at least about 70% identity to the nucleotide sequence set forth in in SEQ ID NO: 155 or 158; and (iv) a nucleic acid encoding a Cas9 enzyme or a functional fragment thereof to a cell or to a subject in need thereof. In some aspects, the method comprises administering to the subject an AAV comprising nucleic acids encoding (i) the DMD gRNAs (one gRNA targeting each of introns 1 and 19), (ii) the DMD donor sequence, (iii) the Cas9 enzyme or a functional fragment thereof. In some aspects, the nucleic acid encoding the Cas9 enzyme comprises the nucleotide sequence set forth in SEQ ID NO: 161, 162, 181, or 183, a variant thereof comprising at least about 70% identity to the nucleotide sequence set forth in in SEQ ID NO: 161, 162, 181, or 183, or a functional fragment thereof. In some aspects, the method comprises administering to the subject the nucleic acids encoding (i) at least two DMD gRNAs, wherein at least one gRNA targets intron 1 and the other gRNA targets intron 19), (ii) the DMD donor sequence, and (iii) the Cas9 enzyme or a functional fragment thereof. In some aspects, the method comprises delivering the nucleic acids in one or more AAV vectors. In some aspects, the method comprises delivering the nucleic acids in non-vectorized delivery.

In yet another aspect, the disclosure provides a method of increasing expression of the DMD gene or increasing the expression of a functional dystrophin in a cell, wherein the method comprises contacting the cell with a nucleic acid comprising (i) a polynucleotide encoding the DMD gRNA comprising the nucleotide sequence set forth in any one of SEQ ID NOs: 1-6, or a variant thereof comprising at least or about 70% identity to the nucleotide sequence set forth in any one of SEQ ID NOs: 1-6; or a polynucleotide encoding a DMD gRNA targeting the nucleotide sequence set forth in any one of SEQ ID NOs: 112-117 (ii) a polynucleotide encoding the DMD gRNA comprising the nucleotide sequence set forth in any one of SEQ ID NOs: 7-9, or a variant thereof comprising at least or about 70% identity to the nucleotide sequence set forth in any one of SEQ ID NOs: 7-9; or a polynucleotide encoding a DMD gRNA targeting the nucleotide sequence set forth in any one of SEQ ID NOs: 118-120 (iii) a donor sequence for replacement of exons 41-55 comprising a polynucleotide comprising the nucleotide sequence set forth in SEQ ID NO: 149 or 152, or 187 or 188, or a variant of any thereof comprising at least about 70% identity to the nucleotide sequence set forth in in SEQ ID NO: 149 or 152, or 187 or 188; and (iv) a nucleic acid encoding a Cas9 enzyme or a functional fragment thereof to a cell or to a subject in need thereof. In some aspects, the method comprises administering to the subject an AAV comprising nucleic acids encoding (i) the DMD gRNAs (one gRNA targeting each of introns 40 and 55), (ii) the DMD donor sequence, (iii) the Cas9 enzyme or a functional fragment thereof. In some aspects, the nucleic acid encoding the Cas9 enzyme comprises the nucleotide sequence set forth in SEQ ID NO: 161, 162, 181, or 183, a variant thereof comprising at least about 70% identity to the nucleotide sequence set forth in SEQ ID NO: 161, 162, 181, or 183, or a functional fragment thereof. In some aspects, the method comprises administering to the subject the nucleic acids encoding (i) at least two DMD gRNAs, wherein at least one gRNA targets intron 40 and the other gRNA targets intron 55), (ii) the DMD donor sequence, and (iii) the Cas9 enzyme or a functional fragment thereof. In some aspects, the method comprises delivering the nucleic acids in one or more AAV vectors. In some aspects, the method comprises delivering the nucleic acids to the cell in non-vectorized delivery.

In yet another aspect, the disclosure provides a method of increasing expression of the DMD gene or increasing the expression of a functional dystrophin in a cell, wherein the method comprises contacting the cell with a nucleic acid comprising (i) a polynucleotide encoding the DMD gRNA comprising the nucleotide sequence set forth in any one of SEQ ID NOs: 10-28, or a variant thereof comprising at least or about 70% identity to the nucleotide sequence set forth in any one of SEQ ID NOs: 10-28; or a polynucleotide encoding a DMD gRNA targeting the nucleotide sequence set forth in any one of SEQ ID NOs: 121-139 (ii) a polynucleotide encoding the DMD gRNA comprising the nucleotide sequence set forth in any one of SEQ ID NOs: 29-37, or a variant thereof comprising at least or about 70% identity to the nucleotide sequence set forth in any one of SEQ ID NOs: 29-37; or a polynucleotide encoding a DMD gRNA targeting the nucleotide sequence set forth in any one of SEQ ID NOs: 140-148 (iii) a donor sequence for replacement of exons 2-19 comprising a polynucleotide comprising the nucleotide sequence set forth in SEQ ID NO: 155 or 158, or a variant thereof comprising at least about 70% identity to the nucleotide sequence set forth in in SEQ ID NO: 155 or 158; and (iv) a nucleic acid encoding a Cas9 enzyme or a functional fragment thereof to a cell or to a subject in need thereof. In some aspects, the method comprises administering to the subject an AAV comprising nucleic acids encoding (i) the DMD gRNAs (one gRNA targeting each of introns 1 and 19), (ii) the DMD donor sequence, (iii) the Cas9 enzyme or a functional fragment thereof. In some aspects, the nucleic acid encoding the Cas9 enzyme comprises the nucleotide sequence set forth in SEQ ID NO: 161, 162, 181, or 183, a variant thereof comprising at least about 70% identity to the nucleotide sequence set forth in in SEQ ID NO: 161, 162, 181, or 183, or a functional fragment thereof. In some aspects, the method comprises administering to the subject the nucleic acids encoding (i) at least two DMD gRNAs, wherein at least one gRNA targets intron 1 and the other gRNA targets intron 19), (ii) the DMD donor sequence, and (iii) the Cas9 enzyme or a functional fragment thereof. In some aspects, the method comprises delivering the nucleic acids in one or more AAV vectors. In some aspects, the method comprises delivering the nucleic acids in non-vectorized delivery.

In some aspects, expression of DMD or the expression of functional dystrophin is increased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

In some aspects, the disclosure provides a recombinant gene editing complex comprising a nucleic acid comprising (i) a polynucleotide encoding the DMD gRNA comprising the nucleotide sequence set forth in any one of SEQ ID NOs: 1-6, or a variant thereof comprising at least or about 70% identity to the nucleotide sequence set forth in any one of SEQ ID NOs: 1-6; or a polynucleotide encoding a DMD gRNA targeting the nucleotide sequence set forth in any one of SEQ ID NOs: 112-117 (ii) a polynucleotide encoding the DMD gRNA comprising the nucleotide sequence set forth in any one of SEQ ID NOs: 7-9, or a variant thereof comprising at least or about 70% identity to the nucleotide sequence set forth in any one of SEQ ID NOs: 7-9; or a polynucleotide encoding a DMD gRNA targeting the nucleotide sequence set forth in any one of SEQ ID NOs: 118-120 (iii) a donor sequence for replacement of exons 41-55 comprising a polynucleotide comprising the nucleotide sequence set forth in SEQ ID NO: 149 or 152, or 187 or 188, or a variant of any thereof comprising at least about 70% identity to the nucleotide sequence set forth in in SEQ ID NO: 149 or 152, or 187 or 188; and (iv) a nucleic acid encoding a Cas9 enzyme or a functional fragment thereof, which are delivered to a cell or to a subject to edit the DMD gene and insert a DMD donor sequence to restore or increase functional dystrophin expression in the cell or in the subject. Such gene editing complex is used for manipulating expression of DMD, increasing functional dystrophin expression, and for treating genetic disease associated with abnormal DMD expression, such as muscular dystrophy, particularly at the RNA level, where disease-relevant sequences, such as those of the DMD gene, are abhorrently expressed.

In some aspects, the disclosure provides a recombinant gene editing complex comprising a nucleic acid comprising (i) a polynucleotide encoding the DMD gRNA comprising the nucleotide sequence set forth in any one of SEQ ID NOs: 10-28, or a variant thereof comprising at least or about 70% identity to the nucleotide sequence set forth in any one of SEQ ID NOs: 10-28; or a polynucleotide encoding a DMD gRNA targeting the nucleotide sequence set forth in any one of SEQ ID NOs: 121-139 (ii) a polynucleotide encoding the DMD gRNA comprising the nucleotide sequence set forth in any one of SEQ ID NOs: 29-37, or a variant thereof comprising at least or about 70% identity to the nucleotide sequence set forth in any one of SEQ ID NOs: 29-37; or a polynucleotide encoding a DMD gRNA targeting the nucleotide sequence set forth in any one of SEQ ID NOs: 140-148 (iii) a donor sequence for replacement of exons 2-19 comprising a polynucleotide comprising the nucleotide sequence set forth in SEQ ID NO: 155 or 158, or a variant thereof comprising at least about 70% identity to the nucleotide sequence set forth in in SEQ ID NO: 155 or 158; and (iv) a nucleic acid encoding a Cas9 enzyme or a functional fragment thereof, which are delivered to a cell or to a subject to edit the DMD gene and insert a DMD donor sequence to restore or increase functional dystrophin expression in the cell or in the subject. Such gene editing complex is used for manipulating expression of DMD, increasing functional dystrophin expression, and for treating genetic disease associated with abnormal DMD expression, such as muscular dystrophy, particularly at the RNA level, where disease-relevant sequences, such as those of the DMD gene, are abhorrently expressed.

In some aspects, the disclosure provides AAV transducing cells for the delivery of nucleic acids encoding the at least two DMD gRNAs (one targeting each of the introns, i.e., 1 and 19, or 40 and 55), the DMD donor sequence, and/or the Cas9 enzyme or a functional fragment thereof. Methods of transducing a target cell with rAAV, in vivo or in vitro, are included in the disclosure. The methods comprise the step of administering an effective dose, or effective multiple doses, of a composition comprising a rAAV of the disclosure to a subject, including an animal (such as a human being) in need thereof. If the dose is administered prior to development of the muscular dystrophy, the administration is prophylactic. If the dose is administered after the development of the muscular dystrophy, the administration is therapeutic. In embodiments of the disclosure, an effective dose is a dose that alleviates (eliminates or reduces) at least one symptom associated with the muscular dystrophy being treated, that slows or prevents progression of the muscular dystrophy, that slows or prevents progression of the muscular dystrophy, that diminishes the extent of disease, that results in remission (partial or total) of the muscular dystrophy, and/or that prolongs survival. In some aspects, the muscular dystrophy is DMD. In some aspects, the muscular dystrophy is BMD.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) are specifically contemplated, as are combinations with other therapies such as those disclosed in International Publication No. WO 2013/016352, which is incorporated by reference herein in its entirety.

Administration of an effective dose of the compositions may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration and serotype(s) of AAV components of rAAV (in particular, the AAV ITRs and capsid protein) of the disclosure may be chosen and/or matched by those skilled in the art taking into account the disease state being treated and the target cells/tissue(s), such as cells that express DMD. In some embodiments, the route of administration is intramuscular. In some embodiments, the route of administration is intravenous.

In particular, actual administration of rAAV of the present disclosure may be accomplished by using any physical method that will transport the rAAV recombinant vector into the target tissue of an animal. Administration according to the disclosure includes, but is not limited to, injection into muscle, the bloodstream, the central nervous system, and/or directly into the brain or other organ. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

The term “transduction” is used to refer to the administration/delivery of one or more of the DMD or Cas9 constructs described herein, including, but not limited to, gRNA, DMD donor sequence, and one or more Cas9-encoding polynucleotides to a recipient cell either in vivo or in vitro, via a replication-deficient rAAV of the disclosure resulting in expression of the DMD gRNAs, DMD donor sequence, and Cas9 by the recipient cell.

In one aspect, transduction with rAAV is carried out in vitro. In one embodiment, desired target cells are removed from the subject, transduced with rAAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells are transduced in vitro by combining rAAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

The disclosure provides methods of administering an effective dose (or doses, administered essentially simultaneously or doses given at intervals) of rAAV that comprise DNA that encodes DMD gRNA and DNA donor sequence, targeted to restore DMD expression, and DNA that encodes Cas9 to effect cleavage and insertion of the DMD donor sequence to a cell or to a subject in need thereof.

Transduction of cells with rAAV of the disclosure results in sustained expression of the guide RNAs targeting DMD expression, DMD donor sequence, and the Cas9 enzyme. The disclosure thus provides methods of administering/delivering rAAV which to restore dystrophin expression to a cell or to a subject. In some aspects, the cell is in a subject. In some aspects, the cell is an animal subject. In some aspects, the animal subject is a human subject.

These methods include transducing the blood and vascular system, the central nervous system, and tissues (including, but not limited to, muscle cells and neurons, tissues, such as muscle, including skeletal muscle, organs, such as heart, brain, skin, eye, and the endocrine system, and glands, such as endocrine glands and salivary glands) with one or more rAAV of the present disclosure. In some aspects, transduction is carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol Cell Biol 11: 4854-4862 (1991)], control elements derived from the human skeletal actin gene [Muscat et al., Mol Cell Biol, 7: 4089-4099 (1987)], the cardiac actin gene, muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors [Semenza et al., Proc. Natl. Acad. Sci. USA, 88: 5680-5684 (1991)], steroid-inducible elements and promoters including the glucocorticoid response element (GRE) [See Mader and White, Proc. Natl. Acad. Sci. USA, 90: 5603-5607 (1993)], the tMCK promoter [see Wang et al., Gene Therapy, 15: 1489-1499 (2008)], the CK6 promoter [see Wang et al., supra] and other control elements.

Because AAV targets every affected organ expressing DMD, the disclosure includes the delivery of DNAs as described herein to all cells, tissues, and organs of a subject. In some aspects, muscle tissue, including skeleton-muscle tissue, is an attractive target for in vivo DNA delivery. The disclosure includes the sustained expression of the DMD gene to express dystrophin from transduced cells. In some aspects, the disclosure includes sustained expression of dystrophin from transduced myofibers. By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells, in some aspects, are differentiated or undifferentiated, such as myoblasts, myocytes, myotubes, cardiomyocytes and cardiomyoblasts.

“Treating” includes ameliorating or inhibiting one or more symptoms of a muscular dystrophy including, but not limited to, muscle wasting, muscle weakness, myotonia, skeletal muscle problems, abnormalities of the retina, hip weakness, facial weakness, abdominal muscle weakness, joint and spinal abnormalities, lower leg weakness, shoulder weakness, hearing loss, muscle inflammation, and nonsymmetrical weakness.

Molecular, biochemical, histological, and functional endpoints demonstrate the therapeutic efficacy of the products and methods disclosed herein for increasing the expression of the DMD gene. Endpoints contemplated by the disclosure include increasing DMD (dystrophin) protein expression, which has application in the treatment of muscular dystrophies including, but not limited to, DMD and BMD and other disorders associated with absent or reduced DMD expression.

The disclosure also provides kits for use in the treatment of a disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Feasibility Studies for HITI Exon Replacement

The HITI methodology described herein above has been utilized not only for the insertion of missing exons and reporter genes at a single site, but also for the replacement of small (˜1.3 kb) portions of the CCAT1 gene in human cancer cells (Zare et al., Biol Proced Online 20, 21, doi:10.1186/s12575-018-0086-5 (2018)). To ensure that a similar approach would work at the DMD locus, previously validated gRNAs targeting up- and down-stream of exon 2 were utilized to remove this exon and subsequently knock in an exogenous DNA sequence (FIG. 3A). From reviewing previously published HITI studies by others, it was not determinable whether larger replacements were feasible using HITI. To this end, a set of previously validated gRNAs, one upstream of exon 2 and one downstream of exon 3, were utilized in a deletion and subsequent HITI experiment to confirm the feasibility of larger genomic replacements than previously described (FIG. 3B).

For the replacement of exon 2 (small replacement, ˜1 kb), two gRNAs flanking this exon were utilized to cut within the genome as well as the HITI donor vector. The cut sites on the donor vector were engineered to be the reverse complement of those cut sites in the genomic context and placed at opposite 5 and 3 ends of one another (FIG. 3A). This was done so that in the case of inverse integration of the HITI donor fragment, the Cas9 cut sites would be reconstituted, allowing for re-cleavage and a greater proportion of integrations being in the forward orientation (FIG. 3A). A similar strategy was used for the replacement of exons 2 and 3 (medium replacement, ˜175 kb) with a gRNA targeting upstream of exon 2 and one downstream of exon 3 (see Table 6), as well as a similarly designed HITI donor fragment (see Table 7) (FIG. 3B).

TABLE 6
gRNA sequences.

| gRNA | | SEQ ID | Human genomic | SEQ ID |
|---|---|---|---|---|
| ID | gRNA sequence | NO: | target sequence | NO: |
| hDSA001 | GAUCAUACAGUAUUUGAA | 165 | ATCATACAGTATTTGAAC | 168 |
| | CGACUGUUUUAGUACUCU | | GACTATGGGT | |
| | GGAAACAGAAUCUACUAA | | | |
| | AACAAGGCAAAAUGCCGU | | | |
| | GUUUAUCUCGUCAACUUG | | | |
| | UUGGCGAGAUUUUU | | | |
| hDSA027 | GCACCCAGCAGAAGAAGA | 166 | CACCCAGCAGAAGAAGA | 169 |
| | UAUGAGUUUUAGUACUCU | | UAUGAGGGAAU | |
| | GGAAACAGAAUCUACUAA | | | |
| | AACAAGGCAAAAUGCCGU | | | |
| | GUUUAUCUCGUCAACUUG | | | |
| | UUGGCGAGAUUUUU | | | |
| JHI3012 | GCUUAGAUUGCUAUUCUA | 167 | CTTAGATTGCTATTCTAA | 170 |
| | AAAAGGUUUUAGUACUCU | | AAAGTAGAGT | |
| | GGAAACAGAAUCUACUAA | | | |
| | AACAAGGCAAAAUGCCGU | | | |
| | GUUUAUCUCGUCAACUUG | | | |
| | UUGGCGAGAUUUUU | | | |

TABLE 7
GFP donor sequence used with hDSA001 and hDSA027 gRNAs.

| SEQ ID | |
|---|---|
| NO: | Donor sequence |
| 171 | ATTCCCTCATATCTTCTTCTGCTGGGTGcaatatgaccgccatgttggcattgattattgactagttattaat |
| | agtaatcaattacggggtcattagttcatagcccatatatggagttccgcgttacataacttacggtaaatggcccgcctggct |
| | gaccgcccaacgacccccgcccattgacgtcaataatgacgtatgttcccatagtaacgccaatagggactttccattgac |
| | gtcaatgggtggagtatttacggtaaactgcccacttggcagtacatcaagtgtatcatatgccaagtccgccccctattgac |
| | gtcaatgacggtaaatggcccgcctggcattatgcccagtacatgaccttacgggactttcctacttggcagtacatctacgt |
| | attagtcatcgctattaccatggtgatgcggttttggcagtacaccaatgggcgtggatagcggtttgactcacggggatttcc |
| | aagtctccaccccattgacgtcaatgggagtttgttttggcaccaaaatcaacgggactttccaaaatgtcgtaataacccc |
| | gccccgttgacgcaaatgggcggtaggcgtgtacggtgggaggtctatataagcagaggtcgtttagtgaaccgtcagat |
| | cactagtagctttattgcggtagtttatcacagttaaattgctaacgcagtcagtgctcgactgatcacaggtaagtatcaagg |
| | ttacaagacaggtttaaggaggccaatagaaactgggcttgtcgagacagagaagattcttgcgtttctgataggcacctat |
| | tggtcttactgacatccactttgcctttctctccacaggggccaccatggAGAGCGACGAGAGCGGCCTGCC |
| | CGCCATGGAGATCGAGTGCCGCATCACCGGCACCCTGAACGGCGTGGAGTTCGA |
| | GCTGGTGGGCGGCGGAGAGGGCACCCCCGAGCAGGGCCGCATGACCAACAAGA |
| | TGAAGAGCACCAAAGGCGCCCTGACCTTCAGCCCCTACCTGCTGAGCCACGTGAT |
| | GGGCTACGGCTTCTACCACTTCGGCACCTACCCCAGCGGCTACGAGAACCCCTTC |
| | CTGCACGCCATCAACAACGGCGGCTACACCAACACCCGCATCGAGAAGTACGAGG |
| | ACGGCGGCGTGCTGCACGTGAGCTTCAGCTACCGCTACGAGGCCGGCCGCGTGA |
| | TCGGCGACTTCAAGGTGATGGGCACCGGCTTCCCCGAGGACAGCGTGATCTTCAC |
| | CGACAAGATCATCCGCAGCAACGCCACCGTGGAGCACCTGCACCCCATGGGCGAT |
| | AACGATCTGGATGGCAGCTTCACCCGCACCTTCAGCCTGCGCGACGGCGGCTACT |
| | ACAGCTCCGTGGTGGACAGCCACATGCACTTCAAGAGCGCCATCCACCCCAGCAT |
| | CCTGCAGAACGGGGGCCCCATGTTCGCCTTCCGCCGCGTGGAGGAGGATCACAG |
| | CAACACCGAGCTGGGCATCGTGGAGTACCAGCACGCCTTCAAGACCCCGGATGCA |
| | GATGCCGGTGAAGAATAAgagatctggatccctcgaggctagcgcggccgcgtttaaacagagctcgatgag |
| | tttggacaaaccacaactagaatgcagtgaaaaaaatgctttatttgtgaaatttgtgatgctattgctttatttgtaaccattat |
| | aagctgcaataaacaagttaacaacaacaattgcattcattttatgtttcaggttcagggggaggACCCATAGTCGTT |
| | CAAATACTGTATGAT |

These experiments utilized a “triple plasmid system” wherein one plasmid had encoded the exogenous knock-in donor DNA sequence flanked by two gRNA cut sites, and two additional plasmids encoded SaCas9 and the two gRNAs utilized in cutting the genomic DNA and the donor plasmid.

Materials and Methods

Molecular Cloning

Generation of the plasmids used in these studies was accomplished through several different traditional and modern cloning techniques. For the swapping of gRNAs, a technique known as restriction free cloning (RFC), which utilizes PCR to amplify the entire plasmid with two mega-primers that contain the desired change flanked by two regions of complementarity to DNA context surrounding the change, was used. All other cloning was accomplished with the In-Fusion cloning kit (Takara Bio) according to the manufacturer's recommendations.

Cell Culture and Treatments

Human embryonic kidney 293 (HEK293) cells were cultured in HEK complete medium (Dulbecco's modified Eagle medium high glucose supplemented with 10% cosmic calf serum, 1% 100× antifungal/antimicrobial and 1% 100× modified Eagle medium non-essential amino acids) in 10 cm² dishes until they were ˜80-90% confluent. Cells were then dissociated from the dishes using 0.025% trypsin-EDTA and counted with a hemacytometer. Cells were plated in each well of a 12-well dish (200,000 cells/well) and allowed to grow for one day before being used for experimentation. Cells were cultured at 37° C. with 100% humidity and 5% CO₂.

Transfection of cells with plasmid DNA was accomplished with the use of Lipofectamine LTX per the manufacturer's recommendations with the indicated amount of DNA, 5 μL of Lipofectamine LTX, and 1 μL of Plus Reagent per μg DNA. Once transfected, cells were incubated for 6 hours before the medium was replaced with fresh HEK complete medium. Cells were cultured for three days before extraction of genomic DNA for analysis.

Genomic DNA PCR Analysis

Polymerase Chain Reaction (PCR) was performed using Q5 Hot Start High-Fidelity 2× master mix per the manufacturer's recommendations with a forward primer that anneals upstream of the 5′ Cas9 cut site and a reverse primer that anneals within the knock-in fragment and addition of 0.08 U/μL of Q5 Hot Start High-Fidelity DNA Polymerase. In the assay performed in carrying out HITI replacement of small and medium sized DMD gene fragments, PCR products were resolved using electrophoresis on either 1% agarose-TAE or 10% polyacrylamide-TBE gels as indicated and stained with ethidium bromide. Images were collected using a BioRad ChemiDoc Imaging System with automatic optimal exposure times. The primers utilized for the PCR differed by assay. In the assay performed in carrying out HITI replacement of small and medium sized DMD gene fragments, the 5′ end amplicon was generated with a forward primer that anneals upstream of the 5′ Cas9 cut site and a reverse primer that anneals within the knock-in fragment. The 3′ end amplicon was generated with a forward primer that anneals within the knock-in fragment and a reverse primer that anneals downstream of the 3′ Cas9 cut site. Finally, in the assay performed in carrying out optimization of HITI plasmid ratios for small and medium sized replacements, the bulk amplicon was generated by the 5′ forward primer and the 3′ reverse primer from the previous assay which encompasses the entire knock-in. Primer Tm's were calculated based on NEB's Q5 DNA polymerase 2× master-mix online suggestions and the extension time was calculated based on the amplicon length, utilizing 30 seconds per kilobase of amplicon.

EnGen® Mutation Detection Assay

The T7E1 assay (EnGen® Mutation Detection Kit; New England Biolabs) used for the confirmation of active gRNAs makes use of the T7 endonuclease I (T7E1) enzyme to cleave at sites of mismatched DNA. To begin, genomic DNA PCR was used to generate amplicons surrounding the expected site of editing. These amplicons were gel purified and subsequently incubated at 95° C., followed by slow annealing at a ramp speed of about 0.1° C./second to allow for the reannealing of heterogeneous DNA indicative of editing. Next, the T7E1 enzyme was added to the reannealed DNA and allowed to incubate at 37° C. for 15 minutes. The resulting cleaved DNA was analyzed via polyacrylamide gel electrophoresis (PAGE) stained with ethidium bromide.

Results and Discussion

Exon 2 and 3 Targeting gRNAs

Prior to the experiments performed in this study, gRNAs targeting exons 2 and 3 of the DMD gene were designed de novo by first identifying SaCas9 PAM sequences (5′-NNGRRT-3′ (SEQ ID NO: 163) within 1000 base pairs (bp) of the targeted exon, because deletion and duplication mutations that affect a given exon have a higher probability of also including the surrounding intronic sequence that is closest to the exon. Importantly, intronic targeting is preferred because the indels that are common with the NHEJ DNA repair pathway are less likely to be deleterious in non-coding regions. It was noted in the design of exon 2 targeting gRNAs that upstream targeting sequences tended to have much larger off-target profiles, likely due to homogeneity of 3′ splice elements, thus the gRNAs were designed both upstream and downstream of exon 2 while they were only designed downstream of exon 3.

Exclusion criteria were used to ensure optimal candidate gRNAs. First, gRNA sequences containing putative RNA polymerase Ill termination signals (four or more contiguous thymidine residues in the coding strand) were excluded, because this could lead to pre-mature termination during transcription from the U6 promoter. Next, gRNAs with more than 30 predicted off-target sites or any number of exonic off-target sites in the human genome, as predicted by CCTop bioinformatics software, were eliminated (Stemmer et al., PLoS One 10, e0124633, doi:10.1371/journal.pone.0124633 (2015)). The predicted off-targets of the remaining gRNAs were noted. This information is being utilized to aid in analyzing off-target profiles. Finally, because mismatches between the target DNA and gRNA or suboptimal PAM sequences can inhibit gene editing, gRNAs were rejected if their target sequence or PAM contained single nucleotide polymorphisms (SNP) or variations (Vars) with greater than one percent minor allele frequency based on the ClinVar and dbSNP databases (Landrum et al., Nucleic Acids Res 46, D1062-D1067, doi:10.1093/nar/gkx1153 (2018); Sherry et al., Nucleic Acids Res 29, 308-311, doi:10.1093/nar/29.1.308 (2001)).

After the initial screening, the candidate gRNAs (see Table 6) were each individually cloned into a plasmid downstream of a U6 promoter along with an SaCas9 expression cassette driven by the cytomegalovirus immediate early enhancer and promoter (CMVP) for high-level, constitutive expression. The plasmids were custom-made by VectorBuilder. These plasmids were used to transfect HEK293 cells to test for efficient cleavage at the expected genomic loci. Amplicons were generated from the genomic DNA flanking the sites of expected editing. The EnGen® Mutation Detection Kit, which makes use of the T7 endonuclease I (T7EI) enzyme that cleaves at sites of DNA mismatches, was used to check for proper editing. These experiments revealed that the lead gRNA candidates were hDSA001 for upstream exon two targeting, hDSA027 for downstream exon two targeting, and JHI3012 for downstream exon three targeting (see Table 6).

HITI Replacement of Small- and Medium-Sized DMD Gene Fragments

To begin, a DNA fragment was used to create a HITI donor vector which contained the genomic Cas9 cut sites on either end of the knock-in fragment as described above (FIG. 3A-B). To test whether or not small- and medium-sized fragments of the DMD gene could be replaced with a HITI donor, HEK293 cells were co-transfected with three plasmids: two containing the SaCas9 expression cassette and the chosen gRNAs, and a HITI donor plasmid containing paired gRNA cut sites flanking a GFP expression cassette. The gRNA pair for the small replacement was hDSA001 and hDSA027, while the gRNA pair for the medium replacement was hDSA001 and JHI3012 (FIG. 3A-B).

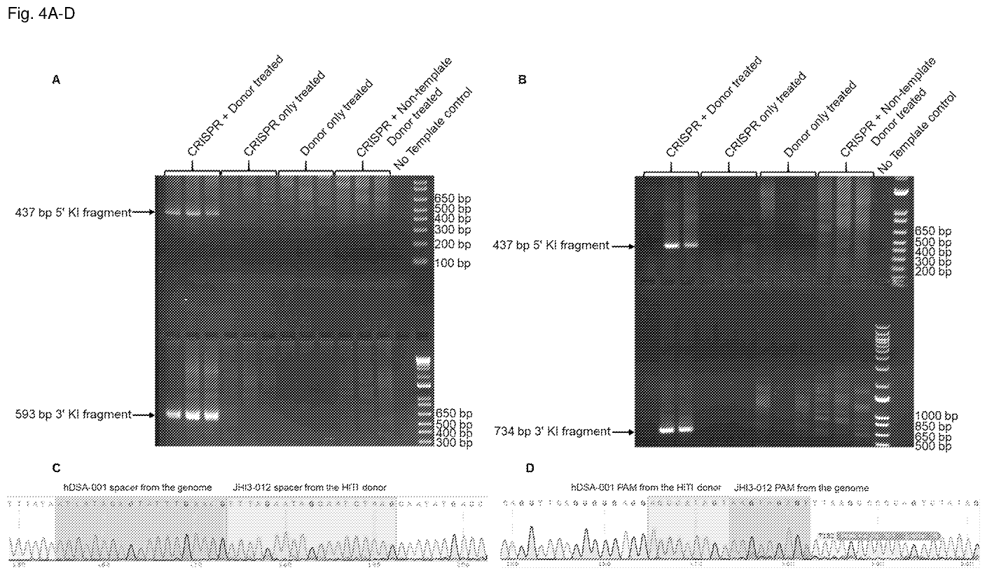

Gel images from genomic DNA PCR for the exon 2 replacement (small replacement of ˜1 kb) revealed that proper integration did occur at the expected locus when using knock-in specific primers (FIG. 4A). A similar result was noted for the replacement of exons 2 and 3 (medium replacement of ˜175 kb) using a similar method for PCR (FIG. 4B). For both experiments, the expected knock-in bands were not present in cells treated with CRISPR only or with donor only showing that these components alone are not sufficient for knock in. The cells treated with a combination of CRISPR and a non-template donor (an identical donor lacking the Cas9 cut sites) also did not show the expected knock-in bands, confirming that Cas9 cleavage of the donor is necessary for HITI knock in to occur. The expected band from the exon 2 and 3 replacement was extracted and sequenced, confirming a seamless integration at the 5 and 3 ends of the insertion (FIGS. 4C and 4D).

Optimization of HITI Plasmid Ratios for Small and Medium Sized Replacements

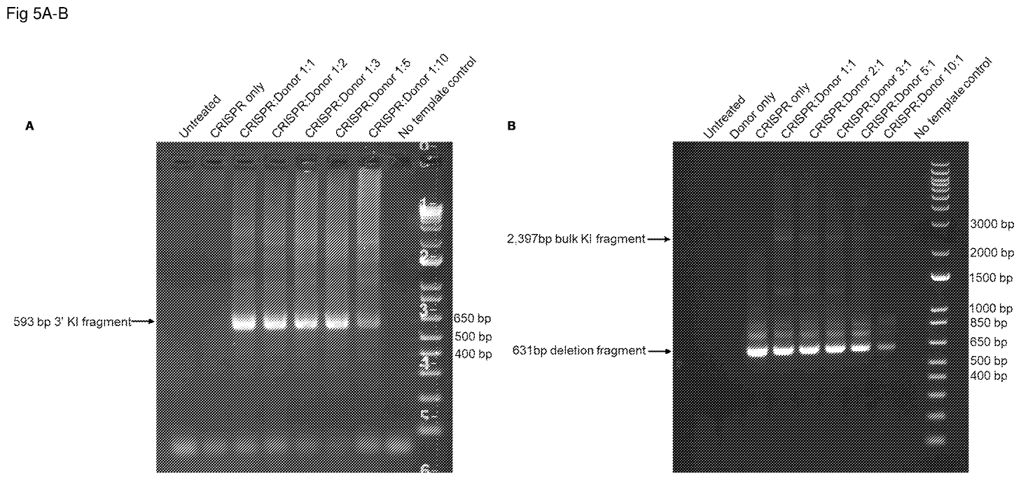

To ensure that the optimal ratios of Cas9 plasmid to Donor plasmid were being used to get the most deletion and integration events possible, variable amounts of Cas9:Donor plasmid were used to transfect HEK293 cells and subsequently screened using the PCR conditions described herein above in FIG. 4A-B. The experiment with decreasing Cas9:Donor ratios was completed with the small replacement and showed that the most integration occurred when there was a 1:1 ratio (FIG. 5A). As this was expected to be applicable regardless of the size of replacement, it was decided that the experiment with increasing Cas9:Donor would be conducted with the medium sized replacement (FIG. 5B). This PCR was also conducted with primers flanking the whole knock-in region instead of the knock-in specific primers to test whether detection of the entire knock-in locus was possible (FIG. 5B). The gel images indicated that the optimal ratio was 1:1 and showed that detection of the whole knock-in amplicon was possible, albeit at a lower efficiency than the knock-in specific primers to test whether detection of the entire knock-in locus was possible (FIG. 5B). The gel images indicated that the optimal ratio was 1:1 and showed that detection of the whole knock-in amplicon was possible, albeit at a lower efficiency than the knock-in specific primers (FIG. 5B). It was postulated that the limiting factor in this experimental set-up is the co-delivery of three plasmids, therefore, a dual-plasmid system was used for subsequent development of this potential therapeutic strategy. The dual-plasmid system comprises one plasmid comprising the knock-in donor sequence flanked on each side of the donor sequences by a genomic Cas9 cut site and two gRNAs; and a second plasmid comprising Cas9 enzyme or a functional fragment thereof capable of generating double-stranded DNA breaks at DNA loci determined by a gRNA spacer sequence.

The experiments carried out in this Example show that the HITI replacement of gene fragments is feasible within the DMD gene, both on a small (˜1 kb) scale and on a larger (˜175 kb) scale via the utilization of SaCas9, two gRNAs and a HITI donor fragment that contains the genomic Cas9 cut sites on either end and in the orientation as described above.

### Example 2

### HITI Replacement of DMD Exons 41-55

Using the basic methodology described in Example 1, a large (˜715 kb) HITI-based gene editing strategy was designed to enable the restoration of full-length dystrophin in a greater number of patients. To this end, bioinformatics analysis was done on the DMD gene to pick an efficient target for exonic replacement. The native locus that contains the introns is ˜715 kb. The synthetic “mega-exon” of e41-55 is ˜2.5 kb (i.e., 2478 bases) and includes only the exons without the introns. The synthetic mega-exon is flanked with synthetic or natural intronic splice sites for inclusion in the spliced transcript.

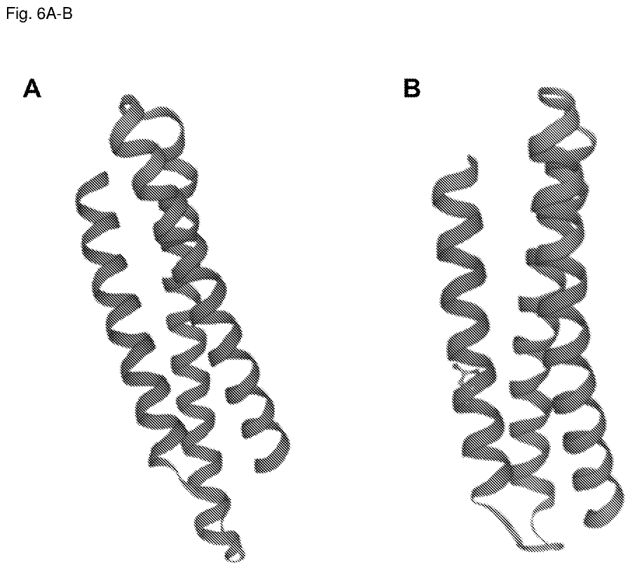

Exons 41-55 encompass two mutational hotspots and efficient replacement of this region could benefit ˜37% of DMD patients. Therefore, this region was the target chosen for experiments described herein in this example (FIG. 1C) (Flanigan et al., Hum Mutat 30, 1657-1666, doi:10.1002/humu.21114 (2009)). In the case that deletion, but not integration, occurs within the region, an open reading frame would be maintained creating a truncated, potentially therapeutic isoform of dystrophin, analogous to the synthetic, miniaturized isoforms of dystrophin, which lack non-essential domains and have been shown to improve symptoms in DMD animal models and is currently being tested in human trials (Bachrach et al., Proc Natl Acad Sci USA 101:3581-3586, doi:10.1073/pnas.0400373101 (2004); Le Guiner et al., Nat Commun 8, 16105, doi:10.1038/ncomms16105 (2017)). Though the excision of 41-55 would truncate two different spectrin-like repeats, SWISS-MODEL online homology-modelling server was used to predict the structure of the resulting hybrid spectrin-like repeat that is formed from the joining of exons 40 and 56. The predicted hybrid spectrin-like repeat modelled on a helical bundle structure similar to the endogenous spectrin-like repeat 22 based on global quality estimates (FIG. 6). Gao et al., Compr Physiol 5, 1223-1239, doi:10.1002/cphy.c140048 (2015); Waterhouse et al., Nucleic Acids Res 46, W296-W303, doi:10.1093/nar/gky427 (2018); Bienert et al., Nucleic Acids Res 45, D313-D319, doi:10.1093/nar/gkw1132 (2017); Guex et al., Electrophoresis 30 Suppl 1, S162-173, doi:10.1002/elps.200900140 (2009); Benkert et al., Bioinformatics 27, 343-350, doi:10.1093/bioinformatics/btq662 (2011); Bertoni et al., Sci Rep 7, 10480, doi:10.1038/s41598-017-09654-8 (2017)).

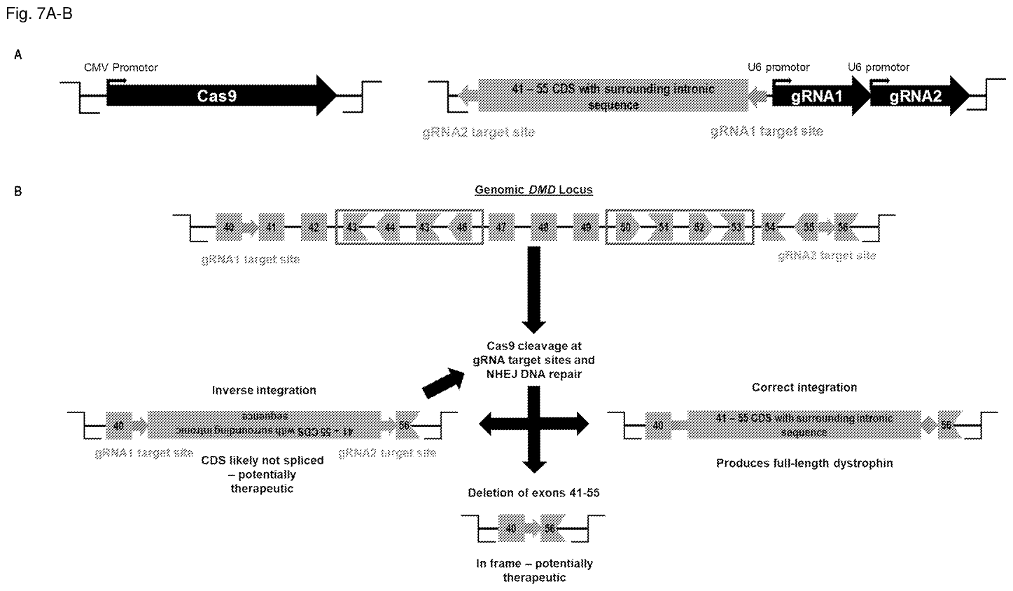

The HITI donor vector was designed such that the coding sequence (CDS) of exons 41-55 (˜2.5 kb) was placed between two regions of ˜100 bp of endogenous intronic sequence to include the 5′ and 3′ splice elements. Just past the intronic sequence on both sides were placed the genomic cut sites, put in the same orientation as described above, once again to reduce the incidence of inverse integration by reconstituting the Cas9 cut sites (FIG. 7). The two gRNAs were included with the HITI donor in one plasmid, allowing for a two-plasmid system wherein there was the HITI donor plasmid with two gRNAs and a plasmid containing the SaCas9 expression cassette.

Materials and Methods

Molecular Cloning

The plasmids for these experiments were constructed through a variety of cloning methods including inverse PCR, wherein primers are used to amplify an entire plasmid except a portion that is to be deleted. These linearized DNA fragments were then used with the In-Fusion cloning kit per the manufacturer's recommendations to create new plasmids.

Cell Culture and Treatments

Culturing of human embryonic kidney 293 (HEK293) cells was accomplished using similar methods as those described herein above in Example 1. Transfections were also accomplished using Lipofectamine LTX as described herein above in Example 1; however, the plasmids used were different for these experiments. The transfections in this Example also used a dual-plasmid transfection system as opposed to the triple plasmid transfection system utilized in Example 1.

Fluorescence Microscopy

Fluorescence microscopy was accomplished by imaging transfected HEK293 cells at room temperature on a Nikon Ti2-E inverted widefield system with a Hamamatsu Orca Flash 4.0 camera. The dimensions of analyzed images were 1022×1024 pixels and were scaled such that there were 1.63 microns/pixel. The fluorescence images were quantified using a custom analysis using the NIS Elements General Analysis 3 module. Cells were identified through automated detection of bright spots of any non-negligible signal intensity after background correction. The mean intensities of red and green signal were then measured and recorded for each bright spot. A threshold based on Otsu methodology was used to differentiate high and low signal in each channel, and spots were counted according to their expression category for each of the two fluorophores. Percent double-positive was calculated by dividing the number of cells with both green and red signal by the total number of cells with both red and green signal, green signal alone, and red signal alone and then multiplying the fraction by 100%.

Genomic DNA PCR Analysis

PCR of extracted HEK293 genomic DNA was accomplished using similar methods as those described in Example 1. Key differences include the primers used, and the cycling conditions. For the experiments conducted in this study, knock-in specific primers were used such that a forward primer was utilized upstream of the genomic Cas9 cut site and a reverse primer was utilized within the HITI knock in. The Tm's were once again calculated based on NEB's Q5 DNA polymerase 2× master-mix online suggestions and the extension time was calculated based on the amplicon length, once again utilizing 30 seconds for every kilobase that was to be amplified.

Results and Discussion

Identification of Lead gRNAs for Replacement of DMD Exons 41-55.

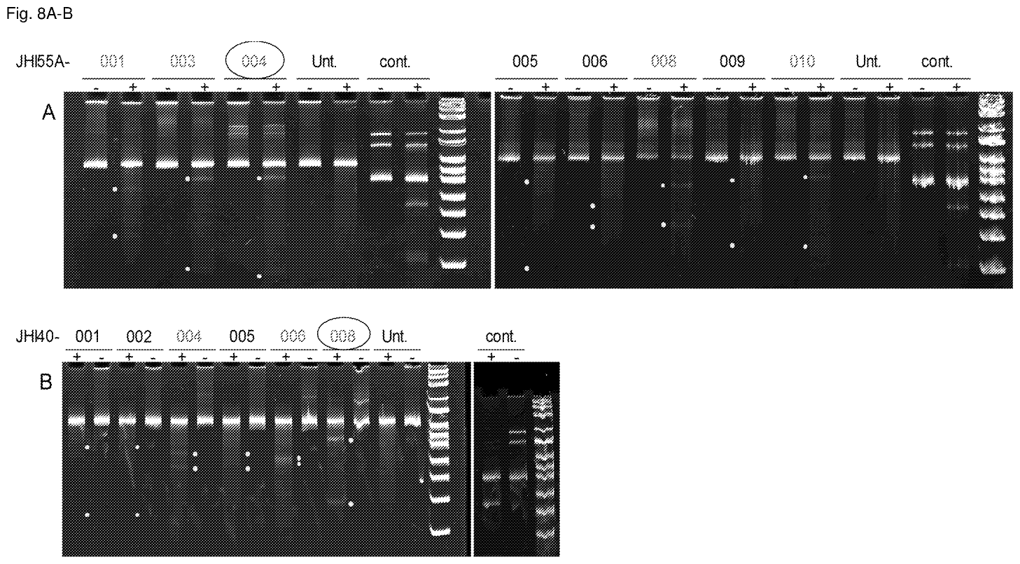

Guide RNAs (gRNAs) targeting upstream of exon 41 (JHI40 series) and downstream of exon 55 (JHI55 series) were designed as described in Example 1. Nine gRNAs that target intron 40 or intron 55 (FIG. 2) were designed by searching the intronic sequences >50 bp downstream of exon 40 and >50 but <1000 bp downstream of exon 55 for the 5′-NNGRRT-3′ (SEQ ID NO: 40) PAM sequence of Sa Cas9. These were subcloned into a plasmid backbone containing a Cas9 expression cassette and then tested for activity in HEK293 cells via lipid-mediated transfection. After 72 hours, a T7 endonuclease I (T7E1) assay (FIG. 8) was used to detect mutations in the unsorted population. From this initial screening, eight active gRNAs were identified that target within intron 40 or intron 55.

Sequences for these gRNAs are set out in Table 1. JHI40-001, JHI40-002, and JHI40-005 gRNAs were found to be inactive in a T7E1 assay (EnGen® Mutation Detection Kit; New England Biolabs); however, additional testing for activity is being determined.

JHI40 series gRNAs targeted within intron 40 as close to exon 40 as possible to include a larger patient cohort (including those with mutations within intron 40). For the JHI55A series gRNAs, an alternative DMD gene promoter exists near the 3′ end of intron 55 and drives expression of an important dystrophin isoform (Dp116) for Schwann cells (Matsuo et al., Genes (Basel) 8, doi:10.3390/genes8100251 (2017)). To avoid removing this alternative promoter, JHI55A gRNAs were designed at the 5 end of intron 55, near exon 55. These gRNAs were cloned into plasmids containing a SaCas9 expression cassette driven by the CMVP with the gRNA driven by a U6 promoter as with the experiments described in Example 1.

The plasmids described above were transfected into HEK293 cells to test the editing capacity of the gRNAs. It was revealed that there were five gRNAs capable of editing from the JHI55A series and three gRNAs were capable of editing from the JHI40 series (FIG. 8). The lead candidates chosen for HITI editing were JHI40-008 and JHI55A-004 (FIG. 8).

Co-Delivery Efficiency

Once the lead gRNAs were chosen, they were cloned into the HITI donor plasmid along with the appropriate Cas9 cut sites on both sides of the donor, while the SaCas9 expression cassette was alone in a separate plasmid. The donor sequence for replacement of exons 41-55 is set out in Table 2.

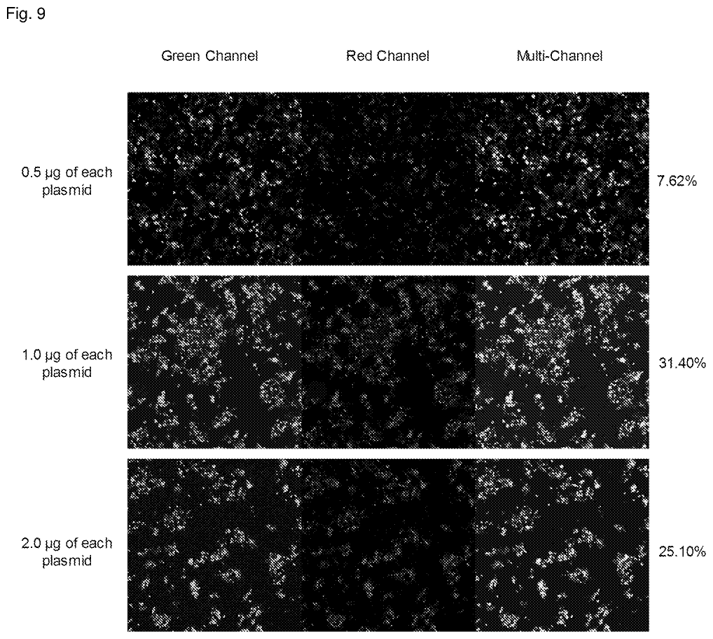

To optimize co-transfection efficiency, the HITI donor plasmid with gRNAs was tagged with a red fluorescence protein (RFP) and the SaCas9 plasmid was tagged with a green fluorescence protein (GFP) and these plasmids were used to co-transfect HEK293 cells using variable amounts of each plasmid at a 1:1 ratio (0.5 μg, 1.0 μg, or 2.0 μg of each plasmid). The cells were imaged using fluorescence microscopy to measure efficiency of co-transfection by the co-localization of RFP and GFP and viability by the estimated percent cell confluency (FIG. 9). Results indicated that the 1.0 μg treated cells had the best co-transfection efficiency with 31.40% double-positive cells; 2.0 μg treated cells had lower efficiency at 25.10% double-positive cells; and the 0.5 μg cells had the lowest efficiency with only 7.62% double-positive cells (FIG. 9).

Detection of HITI Knock in with the Dual-Plasmid System

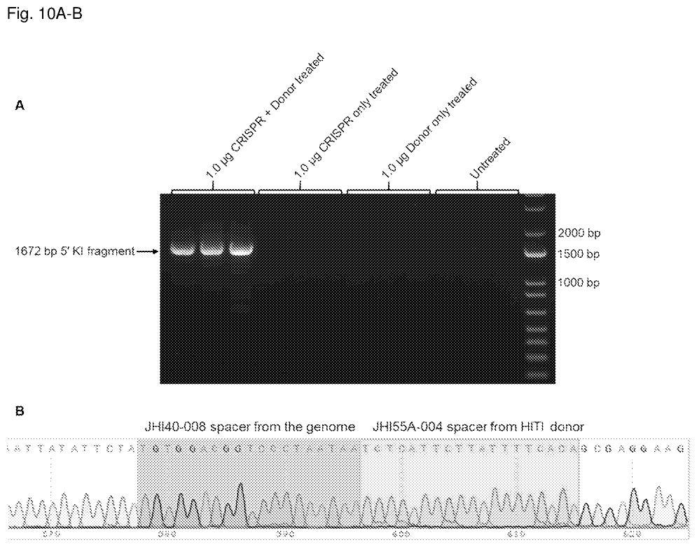

Experiments were then carried out to determine whether the dual-plasmid system resulted in proper HITI knock in. Using genomic DNA extracted from the 1.0 μg treated cells as described herein above, PCR was performed using knock-in specific primers. The results showed successful integration of the HITI donor in the CRISPR and Donor treated cells (FIG. 10A). The CRISPR only treatment and Donor only treatment did not have the expected knock-in band, once again showing that both components are necessary for HITI mediated knock in (FIG. 10A). The untreated cells also did not show the expected knock in band, confirming the lack of knock-in band within the endogenous genome (FIG. 10A). The knock-in band was sequenced, and the sequencing revealed seamless integration (FIG. 10B).

These experiments began by designing and confirming gRNAs that targeted an excision of exons 41 through 55, which were subsequently used in experiments for the replacement of those exons using HITI gene editing. The amount of DNA to be transfected was subsequently optimized in HEK293 cells with the new dual-plasmid HITI system to be 1.0 μg. The resulting genomic DNA was utilized in a PCR which showed the successful integration of the HITI donor into the genome. These experiments lay the groundwork for a therapy to restore full-length dystrophin while simultaneously reaching a diverse array of DMD-causing mutations occurring within exons 41-55.

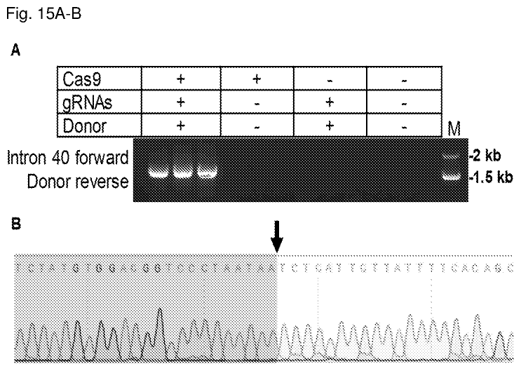

In another experiment, three plasmids were co-delivered using lipid-mediated transfection into HEK293 cells. Two of the plasmids encoded CMV promoter-driven SaCas9 and one of U6 promoter-driven JHI40-008 or JHI55A-004. The third plasmid encoded the DMD exon 41-55 CDS flanked by 100 bp of the native intronic sequences and bookended by the JHI40-008 and JHI55A-004 cut sites (similar to the donor AAV genome in FIG. 11). After 72 hours, PCR was performed using genomic DNA from the unsorted population. Robust amplicons (˜1.6 kb in size) corresponding to the replacement of this 715 kb region with the ˜2.5 kb exon 41-55 CDS encoded in the donor DNA plasmid (FIG. 15A) and was confirmed by sequencing of the amplicons (FIG. 15B).

This study establishes the ability of CRISPR/Cas9 used with the HITI methodology to replace large regions of genomic DNA, which has previously not been explored. By establishing this precedent for such large replacements, the groundwork is set for a DMD therapy which restores full-length dystrophin to a vast cohort of patients with diverse mutations. This study establishes a successful HITI approach to replace the natural DMD exon 41-55 locus (˜715 kb) with a single synthetic exon of ˜2.7 kb that includes the exon 41-55 coding sequence flanked by intronic elements required for splicing which would correct ˜37% of DMD patient mutations. Using plasmid transfection in human cells, this study shows that HITI-mediated replacement of DMD exons 41-55 with a synthetic coding sequence is feasible and warrants translational development to determine in vivo efficiency of gene correction and expression of the resultant transcript.

### Example 3

### HITI Replacement of DMD Exons 2-19

Using the basic methodology described in Examples 1 and 2, a large HITI-based gene editing strategy was designed to replace exons 2-19 of the DMD gene. For exon 2-19 replacement, all potential Sa Cas9 and Cj Cas9 PAM sites (5′-NNGRRT-3′ (SEQ ID NO: 163) and 5′-NNNNRYAC-3′ (SEQ ID NO: 164), respectively) within intron 1 and intron 19 were searched. The full 28 or 30 bp target site sequences of these PAMs were then collected and were aligned to find identical sequences in the mouse Dmd intron 1 and intron 19 to generate DSAi1, DCJi1, DSAi19, and DCJi19 gRNAs, as set out in Table 3. Thus, the gRNAs in Table 3 were designed to target the same intronic regions of intron 1 or intron 19 in both the mouse and human, thus enabling translation of therapy from mice to humans. These gRNAs were cloned and tested as described for JHI40 and JHI55A series gRNAs, described herein above in Example 2. The gRNA sequences that target human DMD introns 1 or 19, and the sequences the gRNAs were designed to target on DMD intron 1 or 19 are provided in Table 3. The donor sequence for exons 2-19 is provided along with other relevant sequences for HITI replacement of exons 2-19 in Table 4.

HEK293 cells (20,000 per well) were plated in a 96-well dish. After 24 hours, cells were treated with lipofection mixes prepared using Lipofectamine LTX and plasmids encoding CMV-driven Sa or Cj Cas9 fused through a T2A peptide to Egfp, as well as a U6 expression cassette for the indicated gRNA. After 72 hours, the cells were re-suspended in TE buffer and ˜10,000 cells were used directly in PCR reactions utilizing primers flanking the gRNA target sites. Amplicons from the PCR reactions were column-purified and sequenced by Sanger sequencing. The sequencing trace files were then analyzed using TIDE software to estimate the editing efficiency and outcomes based upon decomposition of sequence traces (Brinkman et al. Easy quantitative assessment of genome editing by sequence trace decomposition).

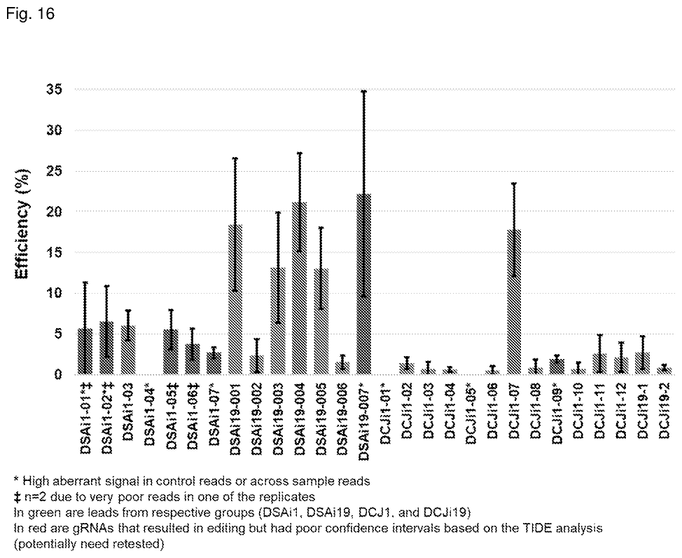

Most gRNAs resulted in editing efficiencies near background levels (<5%) and thus were not considered as leads. Several gRNAs exhibited robust editing (>15% editing efficiency) at the targeted loci of the DMD gene in HEK293 cells. Some sequencing traces resulted in high aberrant base calls throughout the trace in the control or at least one of the test samples (red bars), likely resulting from poor quality amplicons. The lead gRNA with the highest editing efficiency above 5% without poor quality reads was chosen from each series (green bars) resulting in DSAi1-3, DSAi19-4, and DCJi1-07 identified as leads from their respective series of intron 1- and intron 19-targeting gRNAs, respectively (FIG. 16).

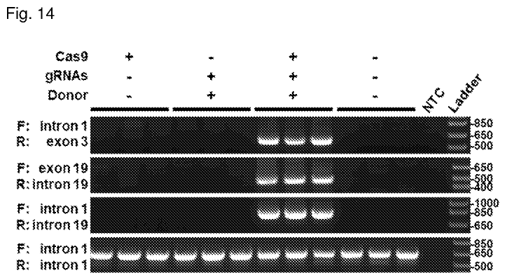

Having determined the active gRNAs, gRNAs designated DSAi1-03 and DSAi19-004 were chosen as target sites for HITI replacement of DMD exons 2-19. HEK293 cells (200,000 cells) were transfected with 1 ug each of two plasmids using Lipofectamine LTX according the manufacturer's suggestions. One plasmid encoded CMVP-driven Sa Cas9 and the other plasmid encoded U6-promoter driven gRNAs DSAi1-03 and DSAi19-004 as well as a HITI donor sequence encoding DMD exons 2-19 (SEQ ID NO: 155) flanked by synthetic splice sites and bookended by the DSAi1-03 and DSAi19-004 target sites. After 72 hours, genomic DNA was extracted and subjected to PCR reactions to detect the gene editing outcomes in the unsorted population (FIG. 14). Robust amplification of the specific knock-in junctions on the 5′ end (intron 1) and 3′ end (intron 19) were detected (FIG. 14). The exon 2-19 deletion-specific amplicon also was detected (FIG. 14).

This study establishes the ability of CRISPR/Cas9 used with the HITI methodology to replace the natural DMD exon 2-19 locus (˜700 kb) with a single synthetic exon of ˜2.5 kb that includes the exon 2-19 coding sequence flanked by intronic elements required for splicing which would correct ˜25% of DMD patient mutations. Using plasmid transfection in human cells, this study shows that HITI-mediated replacement of DMD exons 2-19 with a synthetic coding sequence is feasible and warrants translational development to determine in vivo efficiency of gene correction and expression of the resultant transcript.

### Example 4

### DMD HITI Editing for In Vivo Experiments

Using a mouse model of DMD containing a knock in of the human DMD gene lacking exon 45 on a mouse Dmd knock-out (mdx) background (huDMDdel45; (Young et al., J Neuromuscul Dis 4:139-145, doi:10.3233/JND-170218 (2017)), experiments are carried out to examine in vivo HITI gene editing. This mouse model is phenotypically identical to mdx mice and expresses no human or mouse dystrophin protein. Control mice are heterozygous huDMD mice which have an intact copy of the human DMD gene and also lack mouse dystrophin (Young et al., supra; Hoen et al., J Biol Chem 283: 5899-5907, doi:10.1074/jbc.M709410200 (2008)). The human DMD gene copy in these mice enables use of gRNAs that target human DMD introns which are not homologous to the corresponding mouse introns.

Numbers of mice and viral dosages described below were determined based on other similar published studies and expertise in translational studies in mice. (Wu et al., Cell Stem Cell 13: 659-662, doi:10.1016/j.stem.2013.10.016 (2013); Min et al., Sci Adv 5, eaav4324, doi:10.1126/sciadv.aav4324 (2019); Young et al., supra; Wein et al., Nat Med 20: 992-1000, doi:10.1038/nm.3628 (2014)).

Two rAAV serotype 9 viruses (rAAV9) encoding i) Cas9 alone (rAAV9-Cas9) and ii) gRNA expression cassettes along with a HITI donor fragment (rAAV9-gRNA-HITI) as shown in FIG. 11 are produced by Nationwide Children's Hospital Viral Vector Core. These two rAAV9s are injected together in huDMDdel45 mice (3-6 mice per treatment group) using up to 10¹² total viral particles for intramuscular (IM) injections into tibialis anterior (TA) muscles and up to 10¹⁴ viral particles for systemic injections. IM injections are carried out bilaterally in TA muscles and systemic injections are carried out via the tail vein. Mice are sacrificed at 2, 4, and 8 weeks after injection for muscle tissue harvest and analysis. For IM injections, only TA muscles are analyzed. For systemic injections, dystrophin re-expression is examined in the heart, TA, gastrocnemius, and diaphragm, which are standard for evaluation of DMD therapies. Gene repair efficiency is measured using previously published methods of quantifying dystrophin expression with end-point RT-PCR, western blot, and immunofluorescence microscopy of tissue cross sections (Wein et al., Nat Med 20: 992-1000, doi:10.1038/nm.3628 (2014)).

Optimal ratios of HITI gene editing components are determined to use during systemic injections. To begin optimizing the ratio of the rAAV9-Cas9 to rAAV9-gRNA-HITI, the required rAAV-gRNA-HITI dose for maximal accumulation in muscle tissue will be determined. rAAV9-gRNA-HITI is systemically injected via tail-vein in 12-week-old huDMDdel45 mice (n=3-6 per group) at three doses of 10¹², 10¹³, and 10¹⁴ viral particles. Mice are sacrificed 2 weeks post-injection for tissue harvest, and vector genome copy numbers are measured via qPCR with a standard curve method to determine the minimal amount of AAV that is required to result in maximal accumulation of the AAV in the analyzed tissue. DNA extracted from heart, TA, gastrocnemius, and diaphragm muscles is analyzed with test and control primer-probe sets against a unique region of the rAAV9-gRNA-HITI genome and a mouse genomic target, respectively. The rAAV9-gRNA-HITI at the measured optimal dose is held constant while titrating the rAAV9-Cas9 at doses of 10¹², 10¹³, and 10¹⁴ viral particles via systemic injection. For example, if 10¹³ is determined to be the minimum dose for maximal tissue accumulation, 10¹³ of donor AAV is mixed with various amounts of the Cas9 AAV virus, and both are injected together in mice to determine the optimal ratio of the two AAVs to result in maximal knock-in efficiency.

Mice are sacrificed at a time point between 2-8 weeks. Dystrophin restoration is measured in the heart, TA, gastrocnemius, and diaphragm with end-point RT-PCR, western blot, and immunofluorescence microscopy of tissue cross sections (Wein et al., Nat Med 20, 992-1000, doi:10.1038/nm.3628 (2014)).

A dose response of HITI-mediated DMD gene repair after systemic rAAV9 injections is measured. A dose response curve is prepared using data collected from huDMDdel45 after systemic injection of the two rAAV9s into the tail-vein of 12-week-old huDMDdel45 mice (n=3-6 per group) at three doses (10¹², 10¹³, and 10¹⁴) of total viral particles. The mice are sacrificed for tissue harvest at various time points, as discussed herein above, and analysis is performed on heart, TA, gastrocnemius, and diaphragm muscles tissues. Dystrophin restoration is measured in the heart, TA, gastrocnemius, and diaphragm by end-point RT-PCR, western blot, and immunofluorescence microscopy of tissue cross sections (Wein (2014), supra).

Full-length dystrophin expression is restored in some muscle fibers of huDMDdel45 mice treated with two rAAV9s encoding HITI gene editing components through replacement of the DMD exon 41-55 locus with a synthetic coding sequence provided by one of the rAAV9 genomes. These results indicate that the disclosure provides a gene therapy strategy for DMD which restores full-length dystrophin expression or functional dystrophin expression.

### Example 5

### DMD HITI Editing for Knock-In Coding Sequence for Exons 1-19

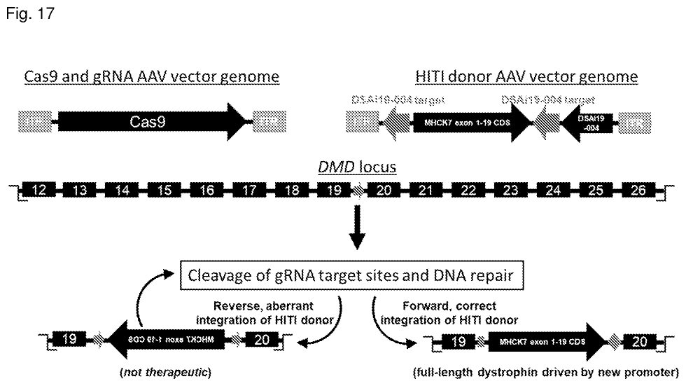

To correct DMD mutations at the 5′ end of the gene, an alternative HITI-mediated strategy is possible which does not rely on replacement of large segments of the DMD gene as described for using donor DNAs with nucleotide sequences set forth in SEQ ID NOs: 149, 152, 187, or 188. In this alternative approach, a donor DNA comprised of a promoter, a synthetic coding sequence of exons 1-19 (without the introns), and a splice donor sequence can be knocked-in within the native intron 19, as depicted in FIG. 17. Thus, the promoter of the knocked-in donor sequence will drive transcription of the synthetic exons 1-19 coding sequence and splice donor as well as the native DMD exons 20-79 of DMD. After splicing of the synthetic exons 1-19 coding sequence and natural exons 20-79, the outcome is full-length dystrophin expression in individuals with virtually any DMD mutation upstream of the intron 19 target site, including mutations within the promoter or 5′ UTR, as well as in any of exon 1 through intron 19.

To this end, a donor DNA sequence was designed to be used with the approach described herein and depicted in FIG. 17. The complete donor sequence comprises donor sequence for knock-in of an MHCK7 promoter followed by DMD Dp427m transcript 5′ untranslated region (UTR) as well as exons 1-19 of the DMD gene. The complete donor sequence thus comprises the DSAi19-004 target site sequence; the MHCK7 promoter sequence; the dp427m 5′ UTR, the DMD exons 1-19 coding sequence modified with a Kozak consensus sequence and an alanine amino acid insertion after the start codon; the downstream intronic fragment containing splice donor site; and a second copy of the DSAi19-004 target site sequence. Thus, the complete donor sequence contains 1) coding sequence of the exons, 2) splice donor intronic elements, and 3) Cas9 target sites on the ends.

As described herein above, the complete donor sequence (SEQ ID NO: 172) comprises the DSAi19-004 genomic target site (SEQ ID NO: 173), the MHCK7 promoter (SEQ ID NO: 174), the 5′ UTR of the dp427m transcript (SEQ ID NO: 175), modified DMD exon 1-19 coding sequence (i.e., Kozak consensus sequence) (SEQ ID NO: 176), a splice donor sequence from human hemoglobin subunit beta gene intron 1 (SEQ ID NO: 177), and a second copy of the DSAi19-004 genomic target site (SEQ ID NO: 178), all as set out in

TABLE 8
Table 8 below.
DNA sequences for DMD HITI Editing for Knock-In Coding Sequence for Exons 1-19.

| SEQ ID | |
|---|---|
| NO: | Sequence of the Complete Donor and Sequences of its Subparts |
| 172 | Complete Donor Sequence |
| | ATCCATTAATTTTATTACTTGTGTACAGGAATTCAAACaagcttgcatgtctaagctagacccttca |
| | gattaaaaataactgaggtaagggcctgggtaggggaggtggtgtgagacgctcctgtctctcctctatctgcccatcggcc |
| | ctttggggaggaggaatgtgcccaaggactaaaaaaaggccatggagccagaggggcgagggcaacagacctttcat |
| | gggcaaaccttggggccctgctgtctagcatgccccactacgggtctaggctgcccatgtaaggaggcaaggcctgggg |
| | acacccgagatgcctggttataattaacccagacatgtggctgcccccccccccccaacacctgctgcctctaaaaataac |
| | cctgtccctggtggatcccctgcatgcgaagatcttcgaacaaggctgtgggggactgagggcaggctgtaacaggcttg |
| | ggggccagggcttatacgtgcctgggactcccaaagtattactgttccatgttcccggcgaagggccagctgtcccccgcc |
| | agctagactcagcacttagtttaggaaccagtgagcaagtcagcccttggggcagcccatacaaggccatggggctggg |
| | caagctgcacgcctgggtccggggtgggcacggtgcccgggcaacgagctgaaagctcatctgctctcaggggcccct |
| | ccctggggacagcccctcctggctagtcacaccctgtaggctcctctatataacccaggggcacaggggctgccctcattc |
| | taccaccacctccacagcacagacagacactcaggagcagccagcggGAATTCATCAGTTACTGTGTT |
| | GACTCACTCAGTGTTGGGATCACTCACTTTCCCCCTACAGGACTCAGATCTGGGAG |
| | GCAATTACCTTCGGAGAAAAACGAATAGGAAAAACTGAAGTGTTACTTTTTTTAAAG |
| | CTGCTGAAGTTTGTTGGTTTCTCATTGTTTTTAAGCCTACTGGAGCAATAAAGTTTG |
| | AAGAACTTTTACCAGGTTTTTTTTATCGCTGCCTTGATATACACTTTTCAAAGCCACC |
| | ATGGCCCTTTGGTGGGAAGAAGTAGAGGACTGTTATGAAAGAGAAGATGTTCAAAA |
| | GAAAACATTCACAAAATGGGTAAATGCACAATTTTCTAAGTTTGGGAAGCAGCATAT |
| | TGAGAACCTCTTCAGTGACCTACAGGATGGGAGGCGCCTCCTAGACCTCCTCGAA |
| | GGCCTGACAGGGCAAAAACTGCCAAAAGAAAAAGGATCCACAAGAGTTCATGCCC |
| | TGAACAATGTCAACAAGGCACTGCGGGTTTTGCAGAACAATAATGTTGATTTAGTG |
| | AATATTGGAAGTACTGACATCGTAGATGGAAATCATAAACTGACTCTTGGTTTGATT |
| | TGGAATATAATCCTCCACTGGCAGGTCAAAAATGTAATGAAAAATATCATGGCTGGA |
| | TTGCAACAAACCAACAGTGAAAAGATTCTCCTGAGCTGGGTCCGACAATCAACTCG |
| | TAATTATCCACAGGTTAATGTAATCAACTTCACCACCAGCTGGTCTGATGGCCTGG |
| | CTTTGAATGCTCTCATCCATAGTCATAGGCCAGACCTATTTGACTGGAATAGTGTG |
| | GTTTGCCAGCAGTCAGCCACACAACGACTGGAACATGCATTCAACATCGCCAGATA |
| | TCAATTAGGCATAGAGAAACTACTCGATCCTGAAGATGTTGATACCACCTATCCAGA |
| | TAAGAAGTCCATCTTAATGTACATCACATCACTCTTCCAAGTTTTGCCTCAACAAGT |
| | GAGCATTGAAGCCATCCAGGAAGTGGAAATGTTGCCAAGGCCACCTAAAGTGACT |
| | AAAGAAGAACATTTTCAGTTACATCATCAAATGCACTATTCTCAACAGATCACGGTC |
| | AGTCTAGCACAGGGATATGAGAGAACTTCTTCCCCTAAGCCTCGATTCAAGAGCTA |
| | TGCCTACACACAGGCTGCTTATGTCACCACCTCTGACCCTACACGGAGCCCATTTC |
| | CTTCACAGCATTTGGAAGCTCCTGAAGACAAGTCATTTGGCAGTTCATTGATGGAG |
| | AGTGAAGTAAACCTGGACCGTTATCAAACAGCTTTAGAAGAAGTATTATCGTGGCTT |
| | CTTTCTGCTGAGGACACATTGCAAGCACAAGGAGAGATTTCTAATGATGTGGAAGT |
| | GGTGAAAGACCAGTTTCATACTCATGAGGGGTACATGATGGATTTGACAGCCCATC |
| | AGGGCCGGGTTGGTAATATTCTACAATTGGGAAGTAAGCTGATTGGAACAGGAAAA |
| | TTATCAGAAGATGAAGAAACTGAAGTACAAGAGCAGATGAATCTCCTAAATTCAAGA |
| | TGGGAATGCCTCAGGGTAGCTAGCATGGAAAAACAAAGCAATTTACATAGAGTTTT |
| | AATGGATCTCCAGAATCAGAAACTGAAAGAGTTGAATGACTGGCTAACAAAAACAG |
| | AAGAAAGAACAAGGAAAATGGAGGAAGAGCCTCTTGGACCTGATCTTGAAGACCTA |
| | AAACGCCAAGTACAACAACATAAGGTGCTTCAAGAAGATCTAGAACAAGAACAAGT |
| | CAGGGTCAATTCTCTCACTCACATGGTGGTGGTAGTTGATGAATCTAGTGGAGATC |
| | ACGCAACTGCTGCTTTGGAAGAACAACTTAAGGTATTGGGAGATCGATGGGCAAAC |
| | ATCTGTAGATGGACAGAAGACCGCTGGGTTCTTTTACAAGACATCCTTCTCAAATG |
| | GCAACGTCTTACTGAAGAACAGTGCCTTTTTAGTGCATGGCTTTCAGAAAAAGAAG |
| | ATGCAGTGAACAAGATTCACACAACTGGCTTTAAAGATCAAAATGAAATGTTATCAA |
| | GTCTTCAAAAACTGGCCGTTTTAAAAGCGGATCTAGAAAAGAAAAAGCAATCCATG |
| | GGCAAACTGTATTCACTCAAACAAGATCTTCTTTCAACACTGAAGAATAAGTCAGTG |
| | ACCCAGAAGACGGAAGCATGGCTGGATAACTTTGCCCGGTGTTGGGATAATTTAGT |
| | CCAAAAACTTGAAAAGAGTACAGCACAGATTTCACAGGCTGTCACCACCACTCAGC |
| | CATCACTAACACAGACAACTGTAATGGAAACAGTAACTACGGTGACCACAAGGGAA |
| | CAGATCCTGGTAAAGCATGCTCAAGAGGAACTTCCACCACCACCTCCCCAAAAGAA |
| | GAGGCAGATTACTGTGGATTCTGAAATTAGGAAAAGGTTGGATGTTGATATAACTG |
| | AACTTCACAGCTGGATTACTCGCTCAGAAGCTGTGTTGCAGAGTCCTGAATTTGCA |
| | ATCTTTCGGAAGGAAGGCAACTTCTCAGACTTAAAAGAAAAAGTCAATGCCATAGA |
| | GCGAGAAAAAGCTGAGAAGTTCAGAAAACTGCAAGATGCCAGCAGATCAGCTCAG |
| | GCCCTGGTGGAACAGATGGTGAATGGTAAGTATCAAGGTTACAAGACAGGTTTAAG |
| | GAATCCATTAATTTTATTACTTGTGTACAG |
| 173 | DSAi19-004 target site sequence |
| | ATCCATTAATTTTATTACTTGTGTACAG |
| 174 | MHCK7 promoter sequence |
| | AAACaagcttgcatgtctaagctagacccttcagattaaaaataactgaggtaagggcctgggtaggggaggtggtgtg |
| | agacgctcctgtctctcctctatctgcccatcggccctttggggaggaggaatgtgcccaaggactaaaaaaaggccatgg |
| | agccagaggggcgagggcaacagacctttcatgggcaaaccttggggccctgctgtctagcatgccccactacgggtct |
| | aggctgcccatgtaaggaggcaaggcctggggacacccgagatgcctggttataattaacccagacatgtggctgcccc |
| | cccccccccaacacctgctgcctctaaaaataaccctgtccctggtggatcccctgcatgcgaagatcttcgaacaaggct |
| | gtgggggactgagggcaggctgtaacaggcttgggggccagggcttatacgtgcctgggactcccaaagtattactgttcc |
| | atgttcccggcgaagggccagctgtcccccgccagctagactcagcacttagtttaggaaccagtgagcaagtcagccct |
| | tggggcagcccatacaaggccatggggctgggcaagctgcacgcctgggtccggggtgggcacggtgcccgggcaac |
| | gagctgaaagctcatctgctctcaggggcccctccctggggacagcccctcctggctagtcacaccctgtaggctcctctat |
| | ataacccaggggcacaggggctgccctcattctaccaccacctccacagcacagacagacactcaggagcagccagc |
| | g |
| 175 | Dp427m 5′ UTR sequence |
| | ATCAGTTACTGTGTTGACTCACTCAGTGTTGGGATCACTCACTTTCCCCCTACAGG |
| | ACTCAGATCTGGGAGGCAATTACCTTCGGAGAAAAACGAATAGGAAAAACTGAAGT |
| | GTTACTTTTTTTAAAGCTGCTGAAGTTTGTTGGTTTCTCATTGTTTTTAAGCCTACTG |
| | GAGCAATAAAGTTTGAAGAACTTTTACCAGGTTTTTTTTATCGCTGCCTTGATATACA |
| | CTTTTCAAA |
| 176 | Kozak consensus sequence-modified DMD exons 1 through 19 coding sequence |
| | GCCACCATGGCCCTTTGGTGGGAAGAAGTAGAGGACTGTTATGAAAGAGAAGATG |
| | TTCAAAAGAAAACATTCACAAAATGGGTAAATGCACAATTTTCTAAGTTTGGGAAGC |
| | AGCATATTGAGAACCTCTTCAGTGACCTACAGGATGGGAGGCGCCTCCTAGACCT |
| | CCTCGAAGGCCTGACAGGGCAAAAACTGCCAAAAGAAAAAGGATCCACAAGAGTT |
| | CATGCCCTGAACAATGTCAACAAGGCACTGCGGGTTTTGCAGAACAATAATGTTGA |
| | TTTAGTGAATATTGGAAGTACTGACATCGTAGATGGAAATCATAAACTGACTCTTGG |
| | TTTGATTTGGAATATAATCCTCCACTGGCAGGTCAAAAATGTAATGAAAAATATCAT |
| | GGCTGGATTGCAACAAACCAACAGTGAAAAGATTCTCCTGAGCTGGGTCCGACAAT |
| | CAACTCGTAATTATCCACAGGTTAATGTAATCAACTTCACCACCAGCTGGTCTGATG |
| | GCCTGGCTTTGAATGCTCTCATCCATAGTCATAGGCCAGACCTATTTGACTGGAAT |
| | AGTGTGGTTTGCCAGCAGTCAGCCACACAACGACTGGAACATGCATTCAACATCGC |
| | CAGATATCAATTAGGCATAGAGAAACTACTCGATCCTGAAGATGTTGATACCACCTA |
| | TCCAGATAAGAAGTCCATCTTAATGTACATCACATCACTCTTCCAAGTTTTGCCTCA |
| | ACAAGTGAGCATTGAAGCCATCCAGGAAGTGGAAATGTTGCCAAGGCCACCTAAA |
| | GTGACTAAAGAAGAACATTTTCAGTTACATCATCAAATGCACTATTCTCAACAGATC |
| | ACGGTCAGTCTAGCACAGGGATATGAGAGAACTTCTTCCCCTAAGCCTCGATTCAA |
| | GAGCTATGCCTACACACAGGCTGCTTATGTCACCACCTCTGACCCTACACGGAGC |
| | CCATTTCCTTCACAGCATTTGGAAGCTCCTGAAGACAAGTCATTTGGCAGTTCATTG |
| | ATGGAGAGTGAAGTAAACCTGGACCGTTATCAAACAGCTTTAGAAGAAGTATTATC |
| | GTGGCTTCTTTCTGCTGAGGACACATTGCAAGCACAAGGAGAGATTTCTAATGATG |
| | TGGAAGTGGTGAAAGACCAGTTTCATACTCATGAGGGGTACATGATGGATTTGACA |
| | GCCCATCAGGGCCGGGTTGGTAATATTCTACAATTGGGAAGTAAGCTGATTGGAAC |
| | AGGAAAATTATCAGAAGATGAAGAAACTGAAGTACAAGAGCAGATGAATCTCCTAA |
| | ATTCAAGATGGGAATGCCTCAGGGTAGCTAGCATGGAAAAACAAAGCAATTTACAT |
| | AGAGTTTTAATGGATCTCCAGAATCAGAAACTGAAAGAGTTGAATGACTGGCTAAC |
| | AAAAACAGAAGAAAGAACAAGGAAAATGGAGGAAGAGCCTCTTGGACCTGATCTTG |
| | AAGACCTAAAACGCCAAGTACAACAACATAAGGTGCTTCAAGAAGATCTAGAACAA |
| | GAACAAGTCAGGGTCAATTCTCTCACTCACATGGTGGTGGTAGTTGATGAATCTAG |
| | TGGAGATCACGCAACTGCTGCTTTGGAAGAACAACTTAAGGTATTGGGAGATCGAT |
| | GGGCAAACATCTGTAGATGGACAGAAGACCGCTGGGTTCTTTTACAAGACATCCTT |
| | CTCAAATGGCAACGTCTTACTGAAGAACAGTGCCTTTTTAGTGCATGGCTTTCAGAA |
| | AAAGAAGATGCAGTGAACAAGATTCACACAACTGGCTTTAAAGATCAAAATGAAATG |
| | TTATCAAGTCTTCAAAAACTGGCCGTTTTAAAAGCGGATCTAGAAAAGAAAAAGCAA |
| | TCCATGGGCAAACTGTATTCACTCAAACAAGATCTTCTTTCAACACTGAAGAATAAG |
| | TCAGTGACCCAGAAGACGGAAGCATGGCTGGATAACTTTGCCCGGTGTTGGGATA |
| | ATTTAGTCCAAAAACTTGAAAAGAGTACAGCACAGATTTCACAGGCTGTCACCACC |
| | ACTCAGCCATCACTAACACAGACAACTGTAATGGAAACAGTAACTACGGTGACCAC |
| | AAGGGAACAGATCCTGGTAAAGCATGCTCAAGAGGAACTTCCACCACCACCTCCC |
| | CAAAAGAAGAGGCAGATTACTGTGGATTCTGAAATTAGGAAAAGGTTGGATGTTGA |
| | TATAACTGAACTTCACAGCTGGATTACTCGCTCAGAAGCTGTGTTGCAGAGTCCTG |
| | AATTTGCAATCTTTCGGAAGGAAGGCAACTTCTCAGACTTAAAAGAAAAAGTCAATG |
| | CCATAGAGCGAGAAAAAGCTGAGAAGTTCAGAAAACTGCAAGATGCCAGCAGATC |
| | AGCTCAGGCCCTGGTGGAACAGATGGTGAATG |
| 177 | Splice donor sequence from human hemoglobin subunit beta gene intron 1 |
| | GTAAGTATCAAGGTTACAAGACAGGTTTAAGGA |
| 178 | Second copy of DSAi19-004 target site sequence |
| | ATCCATTAATTTTATTACTTGTGTACAG |

The DSAi19-004 target sites in the donor DNA are reverse complements of the native target site in DMD intron 19 such that inverse knock-in will reconstitute the target sites and enable re-cleavage by Cas9 to remove the inverted knocked in and potentially drive the desired knock-in orientation (FIG. 17). The MHCK7 promoter was chosen for its strong muscle-specific expression and an alanine amino acid insertion was added after the start codon to install a Kozak consensus sequence at the start codon to drive efficient translation. This approach differs from the other two described herein as no genomic DNA deletions occur to result in the desired outcome. Instead, the donor DNA is knocked-in within intron 19 and includes its own promoter to drive expression of full-length dystrophin.

Using neonatal dystrophic mice carrying an exon 2 duplication mutation, experiments are carried out to examine in vivo HITI gene editing to knock-in coding sequence form DMD. Two rAAV serotype 9 viruses (rAAV9) encoding i) MHCK7-driven Cas9 (rAAV9-Cas9) and ii) a DSAi19-004 gRNA expression cassette along with the HITI donor fragment comprising SEQ ID NO: 172 (rAAV9-gRNA-HITI) as shown in Table 8 and FIG. 17 are produced by Nationwide Children's Hospital Viral Vector Core. These two rAAV9s are injected together in dup2 neonatal mice (3-6 mice per treatment group) using up to 10¹² total viral particles for intramuscular (IM) injections into tibialis anterior (TA) muscles and up to 10¹⁴ viral particles for systemic injections. IM injections are carried out bilaterally in TA muscles and systemic injections are carried out via the tail vein or intraperitoneally. Mice are sacrificed at 4 weeks after injection for muscle tissue harvest and analysis. For IM injections, only TA muscles are analyzed. For systemic injections, dystrophin re-expression is examined in the heart, TA, gastrocnemius, and diaphragm, which are standard for evaluation of DMD therapies. Gene repair efficiency is measured using previously published methods of quantifying dystrophin expression with digital PCR, quantitative PCR, end-point RT-PCR, western blot, and immunofluorescence microscopy of tissue cross sections (Wein et al., Nat Med 20: 992-1000, doi:10.1038/nm.3628 (2014)).

Full-length dystrophin expression is restored in some muscle fibers of dup2 mice treated with two rAAV9s encoding HITI gene editing components to knock in an MHCK7-promoter driven DMD exons 1-19 coding sequence provided by one of the rAAV9 genomes. These results indicate that the disclosure provides a gene therapy strategy for DMD which restores full-length dystrophin expression or functional dystrophin expression.

### Example 6

### Modifying Cas9 for Nuclear Localization

Based on preliminary in vitro studies, it has been identified that Cas9 does not fully localized to the nucleus in HEK293 cells despite an N-terminal NLS of simian virus 40 large T antigen and a C-terminal nucleoplasmin nuclear localization sequence (NLS). Thus, efficiency, in some aspects, may be improved by improving nuclear localization.

To improve the efficiency of Cas9 cleavage, an NLS was engineered to be fused to the Cas9 encoding sequence based on previous work with DNA polymerase lambda (PolL). The modular 36 amino acid NLS was designed to be fused to Cas9 on its N-terminus. This NLS module was determined to drive robust nuclear localization when fused to other proteins and, therefore, is fused to Cas9 to improve Cas9 cleavage.

Table 9 provides the DNA sequence encoding the nuclear localization sequence (SEQ ID NO: 179), the amino acid sequence of the nuclear localization sequence (SEQ ID NO: 180), the DNA sequences for S. aureus Cas9 and C. jejuni Cas9 comprising the nuclear localization sequence (SEQ ID NOs: 181 and 183, respectively), and the amino acid sequences for S. aureus Cas9 and C. jejuni Cas9 comprising the nuclear localization sequence (SEQ ID NOs: 182 and 184, respectively).

TABLE 9
DNA and amino acid sequences for nuclear localization of Cas9.

| SEQ ID NO: | Sequence Descriptor and Sequence |
|---|---|
| 179 | hPoIL-NLS_DNAseq |
| | ATGGCCGATCCCAGGGGTATCTTGAAGGCATTTCCCAAGCGGCAGA |
| | AAATTCATGCTGATGCATCATCAAAAGTACTTGCAAAGATTCCTAGGA |
| | GGGAAGAGGGAGAAGAA |
| 180 | hPoIL-NLS_AAseq |
| | MADPRGILKAFPKRQKIHADASSKVLAKIPRREEGEE |
| 181 | SaCas9-hPoIL-NLS_DNAseq |
| | ATGGCCGATCCCAGGGGTATCTTGAAGGCATTTCCCAAGCGGCAGA |
| | AAATTCATGCTGATGCATCATCAAAAGTACTTGCAAAGATTCCTAGGA |
| | GGGAAGAGGGAGAAGAAAAGCGGAACTACATCctgggcctggacatcggcatc |
| | accagcgtgggctacggcatcatcgactacgagacacgggacgtgatcgatgccggcgtgcggctg |
| | ttcaaagaggccaacgtggaaaacaacgagggcaggcggagcaagagaggcgccagaaggct |
| | gaagcggcggaggcggcatagaatccagagagtgaagaagctgctgttcgactacaacctgctga |
| | ccgaccacagcgagctgagcggcatcaacccctacgaggccagagtgaagggcctgagccaga |
| | agctgagcgaggaagagttctctgccgccctgctgcacctggccaagagaagaggcgtgcacaac |
| | gtgaacgaggtggaagaggacaccggcaacgagctgtccaccaaagagcagatcagccggaac |
| | agcaaggccctggaagagaaatacgtggccgaactgcagctggaacggctgaagaaagacggc |
| | gaagtgcggggcagcatcaacagattcaagaccagcgactacgtgaaagaagccaaacagctgc |
| | tgaaggtgcagaaggcctaccaccagctggaccagagcttcatcgacacctacatcgacctgctgg |
| | aaacccggcggacctactatgagggacctggcgagggcagccccttcggctggaaggacatcaaa |
| | gaatggtacgagatgctgatgggccactgcacctacttccccgaggaactgcggagcgtgaagtacg |
| | cctacaacgccgacctgtacaacgccctgaacgacctgaacaatctcgtgatcaccagggacgaga |
| | acgagaagctggaatattacgagaagttccagatcatcgagaacgtgttcaagcagaagaagaag |
| | cccaccctgaagcagatcgccaaagaaatcctcgtgaacgaagaggatattaagggctacagagt |
| | gaccagcaccggcaagcccgagttcaccaacctgaaggtgtaccacgacatcaaggacattaccg |
| | cccggaaagagattattgagaacgccgagctgctggatcagattgccaagatcctgaccatctacca |
| | gagcagcgaggacatccaggaagaactgaccaatctgaactccgagctgacccaggaagagatc |
| | gagcagatctctaatctgaagggctataccggcacccacaacctgagcctgaaggccatcaacctg |
| | atcctggacgagctgtggcacaccaacgacaaccagatcgctatcttcaaccggctgaagctggtgc |
| | ccaagaaggtggacctgtcccagcagaaagagatccccaccaccctggtggacgacttcatcctga |
| | gccccgtcgtgaagagaagcttcatccagagcatcaaagtgatcaacgccatcatcaagaagtacg |
| | gcctgcccaacgacatcattatcgagctggcccgcgagaagaactccaaggacgcccagaaaatg |
| | atcaacgagatgcagaagcggaaccggcagaccaacgagcggatcgaggaaatcatccggacc |
| | accggcaaagagaacgccaagtacctgatcgagaagatcaagctgcacgacatgcaggaaggc |
| | aagtgcctgtacagcctggaagccatccctctggaagatctgctgaacaaccccttcaactatgaggt |
| | ggaccacatcatccccagaagcgtgtccttcgacaacagcttcaacaacaaggtgctcgtgaagca |
| | ggaagaaaacagcaagaagggcaaccggaccccattccagtacctgagcagcagcgacagcaa |
| | gatcagctacgaaaccttcaagaagcacatcctgaatctggccaagggcaagggcagaatcagca |
| | agaccaagaaagagtatctgctggaagaacgggacatcaacaggttctccgtgcagaaagacttca |
| | tcaaccggaacctggtggataccagatacgccaccagaggcctgatgaacctgctgcggagctactt |
| | cagagtgaacaacctggacgtgaaagtgaagtccatcaatggcggcttcaccagctttctgcggcgg |
| | aagtggaagtttaagaaagagcggaacaaggggtacaagcaccacgccgaggacgccctgatca |
| | ttgccaacgccgatttcatcttcaaagagtggaagaaactggacaaggccaaaaaagtgatggaaa |
| | accagatgttcgaggaaaagcaggccgagagcatgcccgagatcgaaaccgagcaggagtaca |
| | aagagatcttcatcaccccccaccagatcaagcacattaaggacttcaaggactacaagtacagcc |
| | accgggtggacaagaagcctaatagagagctgattaacgacaccctgtactccacccggaaggac |
| | gacaagggcaacaccctgatcgtgaacaatctgaacggcctgtacgacaaggacaatgacaagct |
| | gaaaaagctgatcaacaagagccccgaaaagctgctgatgtaccaccacgacccccagacctacc |
| | agaaactgaagctgattatggaacagtacggcgacgagaagaatcccctgtacaagtactacgagg |
| | aaaccgggaactacctgaccaagtactccaaaaaggacaacggccccgtgatcaagaagattaag |
| | tattacggcaacaaactgaacgcccatctggacatcaccgacgactaccccaacagcagaaacaa |
| | ggtcgtgaagctgtccctgaagccctacagattcgacgtgtacctggacaatggcgtgtacaagttcgt |
| | gaccgtgaagaatctggatgtgatcaaaaaagaaaactactacgaagtgaatagcaagtgctatga |
| | ggaagctaagaagctgaagaagatcagcaaccaggccgagtttatcgcctccttctacaacaacgat |
| | ctgatcaagatcaacggcgagctgtatagagtgatcggcgtgaacaacgacctgctgaaccggatc |
| | gaagtgaacatgatcgacatcacctaccgcgagtacctggaaaacatgaacgacaagaggccccc |
| | caggatcattaagacaatcgcctccaagacccagagcattaagaagtacagcacagacattctggg |
| | caacctgtatgaagtgaaatctaagaagcaccctcagatcatcaaaaagggcaaaaggccggcgg |
| | ccacgaaaaaggccggccaggcaaaaaagaaaaagTAA |
| 182 | SaCas9-hPoIL-NLS_AAseq |
| | MADPRGILKAFPKROKIHADASSKVLAKIPRREEGEEKRNYILGLDIGITS |
| | VGYGIIDYETRDVIDAGVRLFKEANVENNEGRRSKRGARRLKRRRRHRI |
| | QRVKKLLFDYNLLTDHSELSGINPYEARVKGLSQKLSEEEFSAALLHLAK |
| | RRGVHNVNEVEEDTGNELSTKEQISRNSKALEEKYVAELQLERLKKDGE |
| | VRGSINRFKTSDYVKEAKQLLKVQKAYHQLDQSFIDTYIDLLETRRTYYE |
| | GPGEGSPFGWKDIKEWYEMLMGHCTYFPEELRSVKYAYNADLYNALND |
| | LNNLVITRDENEKLEYYEKFQIIENVFKQKKKPTLKQIAKEILVNEEDIKGY |
| | RVTSTGKPEFTNLKVYHDIKDITARKEIIENAELLDQIAKILTIYQSSEDIQE |
| | ELTNLNSELTQEEIEQISNLKGYTGTHNLSLKAINLILDELWHTNDNQIAIF |
| | NRLKLVPKKVDLSQQKEIPTTLVDDFILSPVVKRSFIQSIKVINAIIKKYGLP |
| | NDIIIELAREKNSKDAQKMINEMQKRNRQTNERIEEIIRTTGKENAKYLIEKI |
| | KLHDMQEGKCLYSLEAIPLEDLLNNPFNYEVDHIIPRSVSFDNSFNNKVL |
| | VKQEENSKKGNRTPFQYLSSSDSKISYETFKKHILNLAKGKGRISKTKKE |
| | YLLEERDINRFSVQKDFINRNLVDTRYATRGLMNLLRSYFRVNNLDVKVK |
| | SINGGFTSFLRRKWKFKKERNKGYKHHAEDALIIANADFIFKEWKKLDKA |
| | KKVMENQMFEEKQAESMPEIETEQEYKEIFITPHQIKHIKDFKDYKYSHR |
| | VDKKPNRELINDTLYSTRKDDKGNTLIVNNLNGLYDKDNDKLKKLINKSP |
| | EKLLMYHHDPQTYQKLKLIMEQYGDEKNPLYKYYEETGNYLTKYSKKDN |
| | GPVIKKIKYYGNKLNAHLDITDDYPNSRNKVVKLSLKPYRFDVYLDNGVY |
| | KFVTVKNLDVIKKENYYEVNSKCYEEAKKLKKISNQAEFIASFYNNDLIKIN |
| | GELYRVIGVNNDLLNRIEVNMIDITYREYLENMNDKRPPRIIKTIASKTQSI |
| | KKYSTDILGNLYEVKSKKHPQIIKKGKRPAATKKAGQAKKKK |
| 183 | CjCas9-hPoIL-NLS_DNAseq |
| | ATGGCCGATCCCAGGGGTATCTTGAAGGCATTTCCCAAGCGGCAGA |
| | AAATTCATGCTGATGCATCATCAAAAGTACTTGCAAAGATTCCTAGGA |
| | GGGAAGAGGGAGAAGAAGCTCGCATACTCGCTTTTGATATTGGAATT |
| | TCATCCATAGGATGGGCATTTTCAGAAAATGATGAACTTAAAGATTGT |
| | GGAGTCAGGATTTTCACAAAAGTAGAGAATCCCAAAACAGGGGAAAG |
| | CCTTGCTCTCCCAAGGAGACTGGCGCGATCCGCAAGGAAACGACTT |
| | GCTAGGCGCAAAGCAAGGTTGAATCATCTTAAACATCTCATTGCTAAT |
| | GAATTTAAACTCAATTATGAAGATTACCAAAGTTTTGATGAATCTTTGG |
| | CTAAAGCGTATAAAGGTAGTCTCATTTCCCCATATGAACTCCGTTTTC |
| | GCGCATTGAATGAACTTCTCTCTAAACAAGATTTTGCTCGTGTCATTC |
| | TTCACATTGCAAAACGTCGCGGTTATGATGATATTAAGAATTCAGATG |
| | ATAAGGAAAAGGGAGCGATTCTCAAAGCTATTAAACAAAATGAGGAG |
| | AAATTGGCTAACTATCAATCTGTCGGAGAATATCTCTATAAGGAATAT |
| | TTCCAAAAGTTTAAGGAAAATTCCAAGGAATTTACAAATGTGCGAAAT |
| | AAGAAGGAGTCCTATGAAAGGTGCATTGCTCAATCCTTTCTCAAAGAC |
| | GAACTCAAACTCATCTTTAAGAAACAAAGGGAATTTGGGTTTAGTTTT |
| | AGTAAGAAGTTTGAAGAGGAAGTATTGTCAGTGGCTTTCTATAAACGG |
| | GCTCTCAAGGACTTTTCTCATCTGGTCGGAAATTGTTCTTTCTTTACG |
| | GATGAAAAGCGGGCACCGAAGAATTCACCACTCGCGTTTATGTTTGT |
| | CGCACTCACTCGCATTATTAATCTCCTCAATAACCTTAAGAATACAGA |
| | AGGAATTCTTTATACAAAAGATGATCTCAATGCGCTGCTTAATGAAGT |
| | TTTGAAGAATGGAACTCTTACTTATAAACAAACAAAGAAGTTGCTTGG |
| | GTTGTCAGATGATTATGAATTCAAAGGAGAGAAAGGTACTTATTTTAT |
| | CGAGTTTAAGAAATATAAAGAGTTTATTAAAGCACTCGGAGAACATAA |
| | TCTCTCCCAAGACGACCTTAATGAAATTGCAAAAGATATTACACTCAT |
| | TAAAGATGAAATAAAACTGAAGAAAGCACTTGCAAAATATGATCTGAA |
| | TCAAAATCAAATCGATTCACTTTCTAAATTGGAGTTTAAAGACCATTTG |
| | AATATTTCTTTCAAAGCACTTAAATTGGTCACACCACTCATGCTTGAG |
| | GGGAAGAAATACGATGAAGCCTGTAATGAGCTTAATTTGAAAGTCGC |
| | TATTAATGAAGATAAGAAGGATTTTCTTCCAGCTTTTAATGAAACCTAT |
| | TATAAAGATGAGGTTACGAATCCGGTTGTCTTGCGAGCAATTAAGGA |
| | ATATAGGAAAGTACTCAACGCTTTGCTCAAGAAGTATGGTAAAGTACA |
| | TAAAATTAATATTGAACTTGCCCGCGAGGTCGGTAAGAATCATTCACA |
| | ACGGGCTAAAATTGAAAAGGAGCAAAATGAAAATTATAAAGCGAAGA |
| | AAGACGCAGAACTCGAGTGTGAAAAGTTGGGCCTCAAAATTAATTCC |
| | AAGAATATACTCAAGCTTCGGCTGTTTAAGGAACAAAAGGAGTTTTGT |
| | GCATATAGTGGAGAGAAAATCAAAATCTCCGATCTTCAAGACGAAAA |
| | GATGCTGGAAATTGACCATATTTATCCATATTCTAGGTCTTTTGATGAT |
| | AGTTATATGAATAAAGTCCTTGTATTTACAAAACAAAACCAGGAGAAA |
| | CTTAACCAAACTCCCTTTGAGGCTTTTGGGAATGATTCCGCAAAATGG |
| | CAAAAGATTGAAGTATTGGCTAAGAATCTCCCGACCAAGAAACAGAA |
| | ACGAATTTTGGATAAGAACTATAAAGATAAAGAGCAGAAGAATTTTAA |
| | AGATAGAAATCTCAATGATACTCGATACATTGCTCGCCTTGTCTTGAA |
| | TTATACCAAAGACTATTTGGACTTTCTCCCCCTCTCAGATGATGAAAA |
| | TACCAAATTGAATGACACTCAAAAGGGATCAAAAGTCCATGTTGAGG |
| | CCAAAAGTGGGATGCTCACTTCCGCACTCCGCCATACGTGGGGATTT |
| | TCCGCAAAAGACAGGAATAATCACCTGCATCATGCTATAGATGCTGTT |
| | ATAATAGCATATGCAAATAATTCCATTGTCAAAGCCTTTTCTGATTTTA |
| | AGAAGGAACAGGAAAGTAATTCTGCAGAATTGTATGCTAAGAAGATTT |
| | CCGAACTCGATTATAAGAATAAAAGAAAATTCTTTGAACCATTTAGTG |
| | GGTTTCGGCAAAAGGTCTTGGACAAAATTGATGAAATATTTGTCAGCA |
| | AACCAGAAAGGAAGAAACCATCCGGAGCGCTTCATGAAGAGACTTTT |
| | CGGAAGGAAGAGGAATTTTATCAAAGCTATGGCGGAAAAGAGGGAGT |
| | TCTTAAAGCGTTGGAGCTCGGTAAAATACGGAAGGTCAATGGTAAAA |
| | TAGTTAAGAACGGGGATATGTTTAGGGTTGATATATTTAAACATAAGA |
| | AAACAAATAAATTTTATGCTGTTCCCATTTATACTATGGACTTTGCATT |
| | GAAAGTCTTGCCGAATAAAGCGGTCGCTAGGTCCAAGAAAGGAGAG |
| | ATTAAAGACTGGATATTGATGGATGAAAACTACGAATTTTGCTTTTCCT |
| | TGTATAAAGATAGCCTGATTTTGATACAAACCAAAGATATGCAGGAAC |
| | CAGAATTTGTTTATTATAATGCGTTTACAAGTAGTACTGTCAGCCTTAT |
| | TGTCTCCAAACATGACAATAAATTTGAAACCCTCAGTAAGAATCAGAA |
| | AATTTTGTTTAAGAATGCGAATGAGAAAGAGGTTATTGCAAAATCCAT |
| | TGGAATTCAAAATTTGAAGGTATTCGAGAAGTATATTGTCAGCGCGCT |
| | CGGAGAGGTTACTAAAGCTGAATTCCGCCAACGCGAAGATTTCAAGA |
| | AAAAAAGGCCGGCGGCCACGAAAAAGGCCGGCCAGGCAAAAAAGAA |
| | AAAGTGA |
| 184 | CjCas9-hPoIL-NLS_AAseq |
| | MADPRGILKAFPKRQKIHADASSKVLAKIPRREEGEEARILAFDIGISSIGW |
| | AFSENDELKDCGVRIFTKVENPKTGESLALPRRLARSARKRLARRKARL |
| | NHLKHLIANEFKLNYEDYQSFDESLAKAYKGSLISPYELRFRALNELLSKQ |
| | DFARVILHIAKRRGYDDIKNSDDKEKGAILKAIKQNEEKLANYQSVGEYLY |
| | KEYFQKFKENSKEFTNVRNKKESYERCIAQSFLKDELKLIFKKQREFGFS |
| | FSKKFEEEVLSVAFYKRALKDFSHLVGNCSFFTDEKRAPKNSPLAFMFV |
| | ALTRIINLLNNLKNTEGILYTKDDLNALLNEVLKNGTLTYKQTKKLLGLSDD |
| | YEFKGEKGTYFIEFKKYKEFIKALGEHNLSQDDLNEIAKDITLIKDEIKLKKA |
| | LAKYDLNQNQIDSLSKLEFKDHLNISFKALKLVTPLMLEGKKYDEACNEL |
| | NLKVAINEDKKDFLPAFNETYYKDEVTNPVVLRAIKEYRKVLNALLKKYG |
| | KVHKINIELAREVGKNHSQRAKIEKEQNENYKAKKDAELECEKLGLKINS |
| | KNILKLRLFKEQKEFCAYSGEKIKISDLQDEKMLEIDHIYPYSRSFDDSYM |
| | NKVLVFTKQNQEKLNQTPFEAFGNDSAKWQKIEVLAKNLPTKKQKRILD |
| | KNYKDKEQKNFKDRNLNDTRYIARLVLNYTKDYLDFLPLSDDENTKLNDT |
| | QKGSKVHVEAKSGMLTSALRHTWGFSAKDRNNHLHHAIDAVIIAYANNSI |
| | VKAFSDFKKEQESNSAELYAKKISELDYKNKRKFFEPFSGFRQKVLDKID |
| | EIFVSKPERKKPSGALHEETFRKEEEFYQSYGGKEGVLKALELGKIRKVN |
| | GKIVKNGDMFRVDIFKHKKTNKFYAVPIYTMDFALKVLPNKAVARSKKGE |
| | IKDWILMDENYEFCFSLYKDSLILIQTKDMQEPEFVYYNAFTSSTVSLIVSK |
| | HDNKFETLSKNQKILFKNANEKEVIAKSIGIQNLKVFEKYIVSALGEVTKAE |
| | FRQREDFKKKRPAATKKAGQAKKKK |

Thus, the Cas9 expression cassette is modified to improve in vivo expression and nuclear localization. Gene editing and dystrophin expression are measured after injection of the modified system in neonatal mice and the results are compared to those obtained in neonatal mice without the modified system (i.e., without the modified Cas9).

The modified system improves the efficiency of Cas9 cleavage and increases the expression of dystrophin. Muscle and heart tissues are analyzed for dystrophin expression using immunofluorescence imaging and western blotting with dystrophin-specific antibodies. DNA extracted from the tissues is analyzed by quantitative PCR assay to measure gene editing efficiency. The expected outcome is higher gene editing efficiency and restoration of dystrophin in dystrophic mice using the HITI system as described herein coupled with the modified Cas9.

### Example 7

### HITI Exon 41-55s Knock-In in Patient-Derived Cells with Exon 45 Deletion

Materials and Methods

Molecular Cloning and AAV Production

AAV plasmids were produced using a commercially available backbone (pAAV-mcs from Cell Biolabs, Inc). An MHCK7-promoter followed by Sa Cas9 were ligated upstream of the human growth hormone polyadenylation signal using EcoRI and XbaI restriction sites. This plasmid was used to produce AAV1-SaCas9. The HITI donor and gRNA AAV plasmid was cloned using the same pAAV-mcs backbone and ligating the HITI donor sequence (SEQID NO: 149) followed by a U6-promoter driven JHI40-008 gRNA cassette and a U6-promoter driven JHI55A-004 gRNA cassette at the XbaI restriction site. This plasmid was used to produce AAV1-HITIe41-55-gRNA. AAV1s were produced by the Nationwide Children's Hospital Viral Vector Core.

Cell Culture and Treatments

Fibroblast cells from a patient harboring exon 45 deletion (del45) were modified with doxycycline-inducible myoblast determination protein 1 (MyoD) at the Nationwide Children's Hospital Cell Line core as previously described [See Chaouch S, et al. Human gene therapy, 20:784-790 (2009)]. Cells were cultured in FM complete medium (Dulbecco's modified Eagle medium high glucose supplemented with 20% fetal bovine serum and 1% 100× antifungal/antimicrobial) in 10 cm² dishes until they were ˜80-90% confluent. Cells were then dissociated from the dishes using 0.025% trypsin-EDTA and counted with a hemacytometer. Cells were plated in each well of a 12-well dish (50,000 cells/well) and allowed to grow to 80% confluence. Cells were then washed with PBS and switched to Myoblast Medium (PromoCell Skeletal Muscle Cell Growth Medium supplemented with 8 ug/mL doxycycline). After three days, cells were switched to Myotube Medium (Skeletal Muscle Differentiation Medium supplemented 8 ug/mL doxycycline) and treated with a 1:1 ratio of AAV1-SaCas9 and AAV1-HITIe41-55-gRNA at a total dose of 4×10⁶ viruses per cell. Culture medium was replaced every 2-3 days and cells were maintained at 37° C. with 100% humidity and 5% CO₂. Cells were harvested after 14 days in Myotube Medium.

RNA Purification

Cells were lysed and homogenized in Trizol reagent according to the manufacturer's suggested protocol. After isolation of the aqueous phase following addition of chloroform, RNA was precipitated by addition of a 1:10 volume ratio of 3M sodium acetate and 1:3 volume ratio of ethanol. Pellets were washed with 70% ethanol and dissolved in water. The samples were treated with 1U of Thermo Scientific™ DNase I, RNase-free (1 U/μL) for 30 min at 37° C. RNA was purified using Zymo RNA Clean & Concentrator kit.

RT-PCR Analysis of DMD Transcripts

RNA (1 μg) was used to generate cDNA with Thermo Scientific™ RevertAid First Strand cDNA Synthesis Kit. The cDNA (90 ng RNA equivalent) was using in 15 μL PCR reactions with primers annealing to DMD exon 43 (5′-AGCTTGATTTCCAATGGGAAAAAGTTAACAA-3′ (SEQ ID NO: 185) and exon 46 (5′-ATCTGCTTCCTCCAACCATAAAAC-3′ (SEQ ID NO: 186) with Q5 Hot Start High-Fidelity 2× master mix. Thermal cycling was performed according to the manufacturer's recommendations. PCR products were analyzed with a 1% agarose-TAE gel stained with ethidium bromide.

Results and Discussion

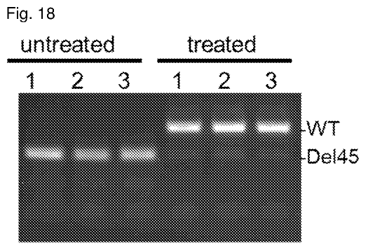

In untreated del45 patient samples, the RT-PCR amplicon size corresponded to the expected size lacking exon 45 (FIG. 18). Treatment with AAV1 encoding the HITI system or replacement of exons 41-55 resulted in robust correction of DMD transcripts to the wild-type size (FIG. 18). Thus, the HITI system, as disclosed herein, efficiently replaced the defective exon 41-55 locus in del45 patient cells with a mega-exon encoding exons 41-55 that was spliced into mature DMD transcripts and resulted in robust restoration of full-length dystrophin.

This study establishes the ability of CRISPR/Cas9 used with the HITI methodology to replace a large region of genomic DNA in a patient-derived cell line. This data further supports the products and methods of the disclosure by demonstrating that the mega-exon encoding DMD exons 41-55 is spliced into mature DMD transcripts. This data further warrants translational development to explore in vivo efficiency of gene correction and expression of full-length dystrophin in vivo.

### Example 8

### Alternative HITI Replacement of DMD Exons 41-55

Most exons in the human genome are <200 bp in length (Sakharkar et al. In Silico Biology 4, 387-393, (2004)). Thus, exon size may influence splicing efficiency. To potentially improve exon recognition and splicing of the DMDe41-55 donor DNA knock-in, an alternative donor DNA sequence was designed. This donor sequence, i.e., the sequence set forth in SEQ ID NO: 187, is used like the donor sequence set forth in SEQ ID NO: 149 (>Complete_DMDe41-55_donor_with_TTN_Introns), described herein above. This donor sequence, i.e., the sequence set forth in SEQ ID NO: 187, contains the DMD exons 41-55 coding sequence divided into individual exons ranging from 190 bp to 378 bp in length and separated by small introns ranging from 86 bp to 142 bp in length from the human titin gene (TTN) transcript isoform N2-B. More specifically, the native intron 40 and 55 splice sites (i.e., SEQ ID NOs: 151 and 153) used in SEQ ID NO: 149 are replaced in SEQ ID NO: 187 (>Complete_DMDe41-55_donor_with_TTN_Introns) by strong branch point, poly-pyrimidine track, and splice acceptor sequences from the human immunoglobulin heavy chain gene intron 1 (>Ig_HC_intron_1_SA_fragment) and the strong splice donor sequence from human β-globin intron 1 (>β-globin_intron_1_SD_fragment). SEQ ID NO: 188 includes only the DMD exon 41-55 coding sequence and introns, while SEQ ID NO: 187 includes the gRNA target sites (similarly to SEQ ID NOs: 149 and 152). The donor sequences (SEQ ID NOs: 187 and 188) and their target sites are set out in Table 10 below.

TABLE 10
Additional DMD donor sequences for TTN DMD exons 41-55 and their
target sites.

| SEQ | Sequence | |
|---|---|---|
| ID NO: | descriptor | Sequence |
| 187 | Complete_ | ATTCAGCCATCTCATTCTTATTTTCACATCTTGCGTTTCTG |
| | DMDe41- | ATAGGCACCTATtggtCTTACTGACATCCACTTTGCCTTTCT |
| | 55_donor_with_ | CTcCaCAGGAAATTGATCGGGAATTGCAGAAGAAGAAAGA |
| | TTN_Introns | GGAGCTGAATGCAGTGCGTAGGCAAGCTGAGGGCTTGTC |
| | | TGAGGATGGGGCCGCAATGGCAGTGGAGCCAACTCAGA |
| | | TCCAGCTCAGCAAGCGCTGGCGGGAAATTGAGAGCAAAT |
| | | TTGCTCAGTTTCGAAGACTCAACTTTGCACAAATTCACAC |
| | | TGTCCGTGAAGAAACGATGATGGTGATGACTGAAGACAT |
| | | GCCTTTGGAAATTTCTTATGTGCCTTCTACTTATTTGACTG |
| | | AAATCACTCATGTCTCACAAGCCCTATTAGAAGTGGAACA |
| | | ACTTCTCAATGCTCCTGACCTCTGTGCTAAGGACTTTGAA |
| | | GATCTCTTTAAGCAAGAGGAGTCTCTGAAGGTATAAAATC |
| | | TTACCTTTTATTCAAATTATAAGTTTTGCGTATGTGTAAAG |
| | | CCAAATAACACACCAAAACACATAAAAGCAAAGCATCGTT |
| | | GGGTTGTCTAAAGCATTATGTTACTTCATCCCTGACCAAT |
| | | ACAGAATATAAAAGATAGTCTACAACAAAGCTCAGGTCGG |
| | | ATTGACATTATTCATAGCAAGAAGACAGCAGCATTGCAAA |
| | | GTGCAACGCCTGTGGAAAGGGTGAAGCTACAGGAAGCTC |
| | | TCTCCCAGCTTGATTTCCAATGGGAAAAAGTTAACAAAAT |
| | | GTACAAGGACCGACAAGGGCGATTTGACAGATCTGTTGA |
| | | GAAATGGCGGCGTTTTCATTATGATATAAAGATATTTAATC |
| | | AGTGGCTAACAGAAGCTGAACAGTTTCTCAGAAAGACACA |
| | | AATTCCTGAGAATTGGGAACATGCTAAATACAAATGGTAT |
| | | CTTAAGGTATGGGGCTTTTAGAATTTGGGGAGGGGTCTC |
| | | AACTTTATTTCACTTCCCTGTGCATTCTGAAAAGCCTCATT |
| | | CTTAATGTCTGATTTTCAGGAACTCCAGGATGGCATTGGG |
| | | CAGCGGCAAACTGTTGTCAGAACATTGAATGCAACTGGG |
| | | GAAGAAATAATTCAGCAATCCTCAAAAACAGATGCCAGTA |
| | | TTCTACAGGAAAAATTGGGAAGCCTGAATCTGCGGTGGC |
| | | AGGAGGTCTGCAAACAGCTGTCAGACAGAAAAAAGAGGC |
| | | TAGAAGAACAAAAGAATATCTTGTCAGAATTTCAAAGAGA |
| | | TTTAAATGAATTTGTTTTATGGTTGGAGGAAGCAGATAACA |
| | | TTGCTAGTATCCCACTTGAACCTGGAAAAGAGCAGCAACT |
| | | AAAAGAAAAGCTTGAGCAAGTCAAGGTAAATGTAACCAAG |
| | | TATAACCAGATAGCCAGTTTCTGAATCATGGGAGTGGGG |
| | | AGTAATAAAATATTTTGCAACCTTTTACTCTTTAATAAACTT |
| | | TAATTTTCACATTCTTCTAATTTTATGCTAAATGTCTTTTAC |
| | | AGTTACTGGTGGAAGAGTTGCCCCTGCGCCAGGGAATTC |
| | | TCAAACAATTAAATGAAACTGGAGGACCCGTGCTTGTAAG |
| | | TGCTCCCATAAGCCCAGAAGAGCAAGATAAACTTGAAAAT |
| | | AAGCTCAAGCAGACAAATCTCCAGTGGATAAAGGTTTCCA |
| | | GAGCTTTACCTGAGAAACAAGGAGAAATTGAAGCTCAAAT |
| | | AAAAGACCTTGGGCAGCTTGAAAAAAAGCTTGAAGACCTT |
| | | GAAGAGCAGTTAAATCATCTGCTGCTGTGGTTATCTCCTA |
| | | TTAGGAATCAGTTGGAAATTTATAACCAACCAAACCAAGA |
| | | AGGACCATTTGACGTTAAGGTGAGTTGCTCAACAATGTAA |
| | | AATTTACCCTATCTGAATCTGCAGTTTATTAGTTCAGTCAT |
| | | GCTAACAAAACTGTATCATTTCAGGAAACTGAAATAGCAG |
| | | TTCAAGCTAAACAACCGGATGTGGAAGAGATTTTGTCTAA |
| | | AGGGCAGCATTTGTACAAGGAAAAACCAGCCACTCAGCC |
| | | AGTGAAGAGGAAGTTAGAAGATCTGAGCTCTGAGTGGAA |
| | | GGCGGTAAACCGTTTACTTCAAGAGCTGAGGGCAAAGCA |
| | | GCCTGACCTAGCTCCTGGACTGACCACTATTGGAGCCTG |
| | | TAAGTCAAGATTAGCTAATTATATAGGAGAGGGGTTGCTT |
| | | GGTTGTGTAGGGTGAAAAAAGGCATAAAATATCTTGATGA |
| | | TTTGTAGGAATAACTATATAAATGATGTTCTTTCTTTCCTT |
| | | CTAACCCTCACTCCAAACAGCTCCTACTCAGACTGTTACT |
| | | CTGGTGACACAACCTGTGGTTACTAAGGAAACTGCCATCT |
| | | CCAAACTAGAAATGCCATCTTCCTTGATGTTGGAGGTACC |
| | | TGCTCTGGCAGATTTCAACCGGGCTTGGACAGAACTTAC |
| | | CGACTGGCTTTCTCTGCTTGATCAAGTTATAAAATCACAG |
| | | AGGGTGATGGTGGGTGACCTTGAGGATATCAACGAGATG |
| | | ATCATCAAGCAGAAGGCAACAATGCAGGATTTGGAACAG |
| | | AGGCGTCCCCAGTTGGAAGAACTCATTACCGCTGCCCAA |
| | | AATTTGAAAAACAAGACCAGCAATCAAGAGGCTAGAACAA |
| | | TCATTACGGATCGAAGTATGCTCTACTTGTCAGCCACGTT |
| | | TTTGTATTTTCTCTGCAAGACTTCCTGATACACCCCTGCAT |
| | | TGATCAAGGGTCATCAATGGAAACGTATTCTGACTTCATC |
| | | CACTGTCCACTTCTTTCAGTTGAAAGAATTCAGAATCAGT |
| | | GGGATGAAGTACAAGAACACCTTCAGAACCGGAGGCAAC |
| | | AGTTGAATGAAATGTTAAAGGATTCAACACAATGGCTGGA |
| | | AGCTAAGGAAGAAGCTGAGCAGGTCTTAGGACAGGCCAG |
| | | AGCCAAGCTTGAGTCATGGAAGGAGGGTCCCTATACAGT |
| | | AGATGCAATCCAAAAGAAAATCACAGAAACCAAGCAGTTG |
| | | GCCAAAGACCTCCGCCAGTGGCAGACAAATGTAGATGTG |
| | | GCAAATGACTTGGCCCTGAAACTTCTCCGGGATTATTCTG |
| | | CAGATGATACCAGAAAAGTCCACATGATAACAGAGAATAT |
| | | CAATGCCTCTTGGAGAAGCATTCATAAAAGGTAAATAGTT |
| | | TTATCAAATAGTCCACCCCAAAATCATTTTTTTTGCCTTTA |
| | | GTTTTATATTTCTTCTTTAAAGTGCTTCAATTAATAAGTTCT |
| | | TTCTTTTTTTTCTTGATAGGGTGAGTGAGCGAGAGGCTGC |
| | | TTTGGAAGAAACTCATAGATTACTGCAACAGTTCCCCCTG |
| | | GACCTGGAAAAGTTTCTTGCCTGGCTTACAGAAGCTGAAA |
| | | CAACTGCCAATGTCCTACAGGATGCTACCCGTAAGGAAA |
| | | GGCTCCTAGAAGACTCCAAGGGAGTAAAAGAGCTGATGA |
| | | AACAATGGCAAGTAAGTATCAAGGTTACAAGACAGGTTTA |
| | | AggaGGCCAATAGAAACTGGGCTTGTCGAGACAGAgAAgA |
| | | TACTCATTATTTATTAGGGACCGTCCACA |
| 188 | DMD exons 41- | TCTTGCGTTTCTGATAGGCACCTATtggtCTTACTGACATCC |
| | 55 coding | ACTTTGCCTTTCTCTcCaCAGGAAATTGATCGGGAATTGCA |
| | sequence with | GAAGAAGAAAGAGGAGCTGAATGCAGTGCGTAGGCAAGC |
| | introns | TGAGGGCTTGTCTGAGGATGGGGCCGCAATGGCAGTGG |
| | | AGCCAACTCAGATCCAGCTCAGCAAGCGCTGGCGGGAAA |
| | | TTGAGAGCAAATTTGCTCAGTTTCGAAGACTCAACTTTGC |
| | | ACAAATTCACACTGTCCGTGAAGAAACGATGATGGTGATG |
| | | ACTGAAGACATGCCTTTGGAAATTTCTTATGTGCCTTCTA |
| | | CTTATTTGACTGAAATCACTCATGTCTCACAAGCCCTATTA |
| | | GAAGTGGAACAACTTCTCAATGCTCCTGACCTCTGTGCTA |
| | | AGGACTTTGAAGATCTCTTTAAGCAAGAGGAGTCTCTGAA |
| | | GGTATAAAATCTTACCTTTTATTCAAATTATAAGTTTTGCG |
| | | TATGTGTAAAGCCAAATAACACACCAAAACACATAAAAGC |
| | | AAAGCATCGTTGGGTTGTCTAAAGCATTATGTTACTTCAT |
| | | CCCTGACCAATACAGAATATAAAAGATAGTCTACAACAAA |
| | | GCTCAGGTCGGATTGACATTATTCATAGCAAGAAGACAGC |
| | | AGCATTGCAAAGTGCAACGCCTGTGGAAAGGGTGAAGCT |
| | | ACAGGAAGCTCTCTCCCAGCTTGATTTCCAATGGGAAAAA |
| | | GTTAACAAAATGTACAAGGACCGACAAGGGCGATTTGAC |
| | | AGATCTGTTGAGAAATGGCGGCGTTTTCATTATGATATAA |
| | | AGATATTTAATCAGTGGCTAACAGAAGCTGAACAGTTTCT |
| | | CAGAAAGACACAAATTCCTGAGAATTGGGAACATGCTAAA |
| | | TACAAATGGTATCTTAAGGTATGGGGCTTTTAGAATTTGG |
| | | GGAGGGGTCTCAACTTTATTTCACTTCCCTGTGCATTCTG |
| | | AAAAGCCTCATTCTTAATGTCTGATTTTCAGGAACTCCAG |
| | | GATGGCATTGGGCAGCGGCAAACTGTTGTCAGAACATTG |
| | | AATGCAACTGGGGAAGAAATAATTCAGCAATCCTCAAAAA |
| | | CAGATGCCAGTATTCTACAGGAAAAATTGGGAAGCCTGAA |
| | | TCTGCGGTGGCAGGAGGTCTGCAAACAGCTGTCAGACAG |
| | | AAAAAAGAGGCTAGAAGAACAAAAGAATATCTTGTCAGAA |
| | | TTTCAAAGAGATTTAAATGAATTTGTTTTATGGTTGGAGGA |
| | | AGCAGATAACATTGCTAGTATCCCACTTGAACCTGGAAAA |
| | | GAGCAGCAACTAAAAGAAAAGCTTGAGCAAGTCAAGGTA |
| | | AATGTAACCAAGTATAACCAGATAGCCAGTTTCTGAATCA |
| | | TGGGAGTGGGGAGTAATAAAATATTTTGCAACCTTTTACT |
| | | CTTTAATAAACTTTAATTTTCACATTCTTCTAATTTTATGCT |
| | | AAATGTCTTTTACAGTTACTGGTGGAAGAGTTGCCCCTGC |
| | | GCCAGGGAATTCTCAAACAATTAAATGAAACTGGAGGACC |
| | | CGTGCTTGTAAGTGCTCCCATAAGCCCAGAAGAGCAAGA |
| | | TAAACTTGAAAATAAGCTCAAGCAGACAAATCTCCAGTGG |
| | | ATAAAGGTTTCCAGAGCTTTACCTGAGAAACAAGGAGAAA |
| | | TTGAAGCTCAAATAAAAGACCTTGGGCAGCTTGAAAAAAA |
| | | GCTTGAAGACCTTGAAGAGCAGTTAAATCATCTGCTGCTG |
| | | TGGTTATCTCCTATTAGGAATCAGTTGGAAATTTATAACCA |
| | | ACCAAACCAAGAAGGACCATTTGACGTTAAGGTGAGTTG |
| | | CTCAACAATGTAAAATTTACCCTATCTGAATCTGCAGTTTA |
| | | TTAGTTCAGTCATGCTAACAAAACTGTATCATTTCAGGAAA |
| | | CTGAAATAGCAGTTCAAGCTAAACAACCGGATGTGGAAG |
| | | AGATTTTGTCTAAAGGGCAGCATTTGTACAAGGAAAAACC |
| | | AGCCACTCAGCCAGTGAAGAGGAAGTTAGAAGATCTGAG |
| | | CTCTGAGTGGAAGGCGGTAAACCGTTTACTTCAAGAGCT |
| | | GAGGGCAAAGCAGCCTGACCTAGCTCCTGGACTGACCAC |
| | | TATTGGAGCCTGTAAGTCAAGATTAGCTAATTATATAGGA |
| | | GAGGGGTTGCTTGGTTGTGTAGGGTGAAAAAAGGCATAA |
| | | AATATCTTGATGATTTGTAGGAATAACTATATAAATGATGT |
| | | TCTTTCTTTCCTTCTAACCCTCACTCCAAACAGCTCCTACT |
| | | CAGACTGTTACTCTGGTGACACAACCTGTGGTTACTAAGG |
| | | AAACTGCCATCTCCAAACTAGAAATGCCATCTTCCTTGAT |
| | | GTTGGAGGTACCTGCTCTGGCAGATTTCAACCGGGCTTG |
| | | GACAGAACTTACCGACTGGCTTTCTCTGCTTGATCAAGTT |
| | | ATAAAATCACAGAGGGTGATGGTGGGTGACCTTGAGGAT |
| | | ATCAACGAGATGATCATCAAGCAGAAGGCAACAATGCAG |
| | | GATTTGGAACAGAGGCGTCCCCAGTTGGAAGAACTCATT |
| | | ACCGCTGCCCAAAATTTGAAAAACAAGACCAGCAATCAAG |
| | | AGGCTAGAACAATCATTACGGATCGAAGTATGCTCTACTT |
| | | GTCAGCCACGTTTTTGTATTTTCTCTGCAAGACTTCCTGAT |
| | | ACACCCCTGCATTGATCAAGGGTCATCAATGGAAACGTAT |
| | | TCTGACTTCATCCACTGTCCACTTCTTTCAGTTGAAAGAAT |
| | | TCAGAATCAGTGGGATGAAGTACAAGAACACCTTCAGAAC |
| | | CGGAGGCAACAGTTGAATGAAATGTTAAAGGATTCAACAC |
| | | AATGGCTGGAAGCTAAGGAAGAAGCTGAGCAGGTCTTAG |
| | | GACAGGCCAGAGCCAAGCTTGAGTCATGGAAGGAGGGT |
| | | CCCTATACAGTAGATGCAATCCAAAAGAAAATCACAGAAA |
| | | CCAAGCAGTTGGCCAAAGACCTCCGCCAGTGGCAGACAA |
| | | ATGTAGATGTGGCAAATGACTTGGCCCTGAAACTTCTCCG |
| | | GGATTATTCTGCAGATGATACCAGAAAAGTCCACATGATA |
| | | ACAGAGAATATCAATGCCTCTTGGAGAAGCATTCATAAAA |
| | | GGTAAATAGTTTTATCAAATAGTCCACCCCAAAATCATTTT |
| | | TTTTGCCTTTAGTTTTATATTTCTTCTTTAAAGTGCTTCAAT |
| | | TAATAAGTTCTTTCTTTTTTTTCTTGATAGGGTGAGTGAGC |
| | | GAGAGGCTGCTTTGGAAGAAACTCATAGATTACTGCAACA |
| | | GTTCCCCCTGGACCTGGAAAAGTTTCTTGCCTGGCTTACA |
| | | GAAGCTGAAACAACTGCCAATGTCCTACAGGATGCTACC |
| | | CGTAAGGAAAGGCTCCTAGAAGACTCCAAGGGAGTAAAA |
| | | GAGCTGATGAAACAATGGCAAGTAAGTATCAAGGTTACAA |
| | | GACAGGTTTAAggaGGCCAATAGAAACTGGGCTTGTCGAG |
| | | ACAGAgAAgAT |
| 150 | JHI55A- | ATTCAGCCATCTCATTCTTATTTTCACA |
| | 004_target_site | |
| 154 | JHI40-008 target | ACTCATTATTTATTAGGGACCGTCCACA |
| | site | |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A nucleic acid encoding a Duchenne muscular dystrophy (DMD) gene-targeting guide RNA (gRNA) comprising:
(a) the nucleotide sequence set forth in any one of SEQ ID NOs: 1-37 or a variant thereof comprising at least or about 80% identity to the nucleotide sequence set forth in any one of SEQ ID NOs: 1-37; or
(b) a nucleotide sequence that specifically hybridizes to a target nucleic acid encoding DMD comprising the nucleotide sequence set forth in any one of SEQ ID NOs: 112-148.

2. A nucleic acid comprising a donor DNA sequence encoding knock-in donor sequence of the DMD gene comprising the nucleotide sequence set forth in SEQ ID NO: 149, 152, 155 158, 172, 176, 187, or 188, or a variant thereof comprising at least or about 80% identity to the nucleotide sequence set forth in SEQ ID NO: 149, 152, 155, 158, 172, 176, 187, or 188.

3. The nucleic acid of claim 1 or 2 further comprising a promoter sequence.

4. The nucleic acid of claim 3, wherein the promoter is any of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

5. The nucleic acid of claim 3 or 4, wherein the promoter is a U6 promoter.

6. A composition comprising the nucleic acid of any one of claims 1-5.

7. A vector comprising the nucleic acid of any one of claims 1-5.

8. The vector of claim 7, wherein the vector is an adeno-associated virus.

9. The adeno-associated virus of claim 8, wherein the virus lacks rep and cap genes.

10. The adeno-associated virus of claim 8 or 9, wherein the virus is a recombinant AAV (rAAV) or a self-complementary AAV (scAAV).

11. The adeno-associated virus of any one of claims 8-10, wherein the virus is AAV1, AAV2, AAV3, AAV4, AAV5, AAV6, AAV7, AAV8, AAV9, AAV10, AAV11, AAV12, AAV13, AAVanc80, or AAVrh.74.

12. The adeno-associated virus of any one of claims 8-11, wherein the virus is rAAV9.

13. A composition comprising the adeno-associated virus of any one of claims 8-12 and a pharmaceutically acceptable carrier.

14. A method for replacing one or more missing, duplicated, aberrant, or aberrantly-spliced exons or missing or aberrant introns in the DMD gene in a cell, the method comprising transfecting the cell with:
a) i) a nucleic acid encoding a first DMD-targeting guide RNA (gRNA) targeting intron 1 and a nucleic acid encoding a second DMD-targeting gRNA targeting intron 19; or
ii) a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19;
b) a nucleic acid comprising a donor DNA sequence encoding knock-in donor sequence of exons 2-19 the DMD gene flanked on each side of the donor sequences by a genomic Cas9 cut site; and
c) a nucleic acid encoding a Cas9 enzyme or a functional fragment thereof.

15. A method for replacing one or more missing, duplicated, aberrant, or aberrantly-spliced exons or missing or aberrant introns in the DMD gene in a cell, the method comprising transfecting the cell with a vector comprising:
a) i) a nucleic acid encoding a first DMD-targeting guide RNA (gRNA) targeting intron 1 and a nucleic acid encoding a second DMD-targeting gRNA targeting intron 19; or
ii) a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19;
b) a nucleic acid comprising a donor DNA sequence encoding knock-in donor sequence of exons 2-19 the DMD gene flanked on each side of the donor sequences by a genomic Cas9 cut site; and
c) a nucleic acid encoding a Cas9 enzyme or a functional fragment thereof.

16. The method of claim 15 or 16, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

17. The method of any one of claims 14-16, wherein the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28.

18. The method of any one of claims 14-16, wherein the nucleic acid encodes a gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139.

19. The method of any one of claims 14-16, wherein the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37.

20. The method of any one of claims 14-16, wherein the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148.

21. The method of any one of claims 14-20, wherein the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158.

22. A method for replacing one or more missing, duplicated, aberrant, or aberrantly-spliced exons or missing or aberrant introns in the DMD gene in a cell, the method comprising transfecting the cell with:
a) i) a nucleic acid encoding a first DMD-targeting guide RNA (gRNA) targeting intron 40 and a nucleic acid encoding a second DMD-targeting gRNA targeting intron 55; or
ii) a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55;
b) a nucleic acid comprising a donor DNA sequence encoding knock-in donor sequence of exons 41-55 of the DMD gene flanked on each side of the donor sequences by a genomic Cas9 cut site; and
c) a nucleic acid encoding a Cas9 enzyme or a functional fragment thereof.

23. A method for replacing one or more missing, duplicated, aberrant, or aberrantly-spliced exons or missing or aberrant introns in the DMD gene in a cell, the method comprising transfecting the cell with a vector comprising:
a) i) a nucleic acid encoding a first DMD-targeting guide RNA (gRNA) targeting intron 40 and a nucleic acid encoding a second DMD-targeting gRNA targeting intron 55; or
ii) a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55;
b) a nucleic acid comprising a donor DNA sequence encoding knock-in donor sequence of exons 41-55 of the DMD gene flanked on each side of the donor sequences by a genomic Cas9 cut site; and
c) a nucleic acid encoding a Cas9 enzyme or a functional fragment thereof.

24. The method of claim 22 or 23, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

25. The method of any one of claims 22-24, wherein the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6.

26. The method of any one of claims 22-24, wherein the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117.

27. The method of any one of claims 22-26, wherein the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9.

28. The method of any one of claims 22-26, wherein the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120.

29. The method of any one of claims 22-28, wherein the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188.

30. The method of any one of claims 22-29, wherein expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

31. The method of any one of claims 14-30, wherein the cell is a human cell.

32. The method of claim 31, wherein the human cell is in a human subject.

33. The method of claim 32, wherein the human subject suffers from a muscular dystrophy.

34. A method of treating a subject suffering from one or more missing, duplicated, aberrant, or aberrantly-spliced exons or missing or aberrant introns in the DMD gene in a cell, the method comprising administering to the subject an effective amount of:
a) i) a nucleic acid encoding a first DMD-targeting guide RNA (gRNA) targeting intron 1 and a nucleic acid encoding a second DMD-targeting gRNA targeting intron 19; or
ii) a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19;
b) a nucleic acid comprising a donor DNA sequence encoding knock-in donor sequence of exons 2-19 the DMD gene flanked on each side of the donor sequences by a genomic Cas9 cut site; and
c) a nucleic acid encoding a Cas9 enzyme or a functional fragment thereof.

35. A method of treating a subject suffering from one or more missing, duplicated, aberrant, or aberrantly-spliced exons or missing or aberrant introns in the DMD gene in a cell, the method comprising administering to the subject an effective amount of a vector comprising:
a) i) a nucleic acid encoding a first DMD-targeting guide RNA (gRNA) targeting intron 1 and a nucleic acid encoding a second DMD-targeting gRNA targeting intron 19; or
ii) a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19;
b) a nucleic acid comprising a donor DNA sequence encoding knock-in donor sequence of exons 2-19 the DMD gene flanked on each side of the donor sequences by a genomic Cas9 cut site; and
c) a nucleic acid encoding a Cas9 enzyme or a functional fragment thereof.

36. The method of claim 34 or 35, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

37. The method of any one of claims 34-36, wherein the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28.

38. The method of any one of claims 34-36, wherein the nucleic acid encoding a first DMD-targeting gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139.

39. The method of any one of claims 34-38, wherein the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37.

40. The method of any one of claims 34-38, wherein the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148.

41. The method of any one of claims 34-40, wherein the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158.

42. A method of treating a subject suffering from one or more missing, duplicated, aberrant, or aberrantly-spliced exons or missing or aberrant introns in the DMD gene in a cell, the method comprising administering to the subject an effective amount of:
a) i) a nucleic acid encoding a first DMD-targeting guide RNA (gRNA) targeting intron 40 and a nucleic acid encoding a second DMD-targeting gRNA targeting intron 55; or
ii) a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55;
b) a nucleic acid comprising a donor DNA sequence encoding knock-in donor sequence of exons 41-55 the DMD gene flanked on each side of the donor sequences by a genomic Cas9 cut site; and
c) a nucleic acid encoding a Cas9 enzyme or a functional fragment thereof.

43. A method of treating a subject suffering from one or more missing, duplicated, aberrant, or aberrantly-spliced exons or missing or aberrant introns in the DMD gene in a cell, the method comprising administering to the subject an effective amount of a vector comprising:
a) i) a nucleic acid encoding a first DMD-targeting guide RNA (gRNA) targeting intron 40 and a nucleic acid encoding a second DMD-targeting gRNA targeting intron 55; or
ii) a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55;
b) a nucleic acid comprising a donor DNA sequence encoding knock-in donor sequence of exons 41-55 the DMD gene flanked on each side of the donor sequences by a genomic Cas9 cut site; and
c) a nucleic acid encoding a Cas9 enzyme or a functional fragment thereof.

44. The method of claim 42 or 43, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

45. The method of any one of claims 42-44, wherein the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6.

46. The method of any one of claims 42-44, wherein the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117.

47. The method of any one of claims 42-46, wherein the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9.

48. The method of any one of claims 42-46, wherein the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120.

49. The method of any one of claims 42-48, wherein the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188.

50. The method of any one of claims 42-49, wherein expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

51. The method of any one of claims 34-50, wherein the subject is a human subject.

52. The method of claim 51, wherein the human subject suffers from a muscular dystrophy.

53. The method of claim 52, wherein the muscular dystrophy is Duchene Muscular Dystrophy (DMD) or Becker Muscular Dystrophy (BMD).

54. A recombinant gene editing complex comprising:
a) i) a nucleic acid encoding a first DMD-targeting guide RNA (gRNA) targeting intron 1 and a nucleic acid encoding a second DMD-targeting gRNA targeting intron 19; or
ii) a nucleic acid encoding a first DMD-targeting gRNA that specifically hybridizes to a target nucleotide sequence in intron 1 and a nucleic acid encoding a second DMD-targeting gRNA that specifically hybridizes to a target nucleotide sequence in intron 19;
b) a nucleic acid comprising a donor DNA sequence encoding knock-in donor sequence of exons 2-19 the DMD gene flanked on each side of the donor sequences by a genomic Cas9 cut site; and
c) a nucleic acid encoding a Cas9 enzyme or a functional fragment thereof, wherein binding of the complex to the target nucleic acid sequence results in increased DMD gene expression.

55. The gene editing complex of claim 54, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

56. The gene editing complex of claim 54 or 55, wherein the nucleic acid encoding a first DMD-targeting gRNA targeting intron 1 comprises the nucleotide sequence set forth in any one of SEQ ID NOs: 10-28 or a variant thereof comprising at least or about 80% identity to the nucleotide sequence set forth in any one of SEQ ID NOs: 10-28.

57. The gene editing complex of claim 54 or 55, wherein the nucleic acid encoding a first DMD-targeting gRNA that specifically hybridizes to a target nucleotide sequence in intron 1 comprising the nucleotide sequence set forth in any one of SEQ ID NOs: 121-139 or a variant thereof comprising at least or about 80% identity to the nucleotide sequence set forth in any one of SEQ ID NOs: 121-139.

58. The gene editing complex of any one of claims 54-57, wherein the nucleic acid encoding a first DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37.

59. The gene editing complex of any one of claims 54-57, wherein the nucleic acid encoding a first DMD-targeting gRNA that specifically hybridizes to a target nucleotide sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148.

60. The gene editing complex of any one of claims 54-59, wherein the nucleic acid encoding the knock-in donor sequence of exons 2-19 comprises the nucleotide sequence set forth in SEQ ID NO: 155 or 158 or a variant thereof comprising at least or about 80% identity to the nucleotide sequence set forth in SEQ ID NO: 155 or 158.

61. The gene editing complex of any one of claims 54-60, wherein the nucleic acid encoding the gRNA or the nucleic acid encoding the Cas9 enzyme further comprises a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

62. The gene editing complex of any one of claims 54-61, wherein the one or more nucleic acids are in a vector.

63. The gene editing complex of claim 62, wherein the vector is AAV.

64. A recombinant gene editing complex comprising:
a) i) a nucleic acid encoding a first DMD-targeting guide RNA (gRNA) targeting intron 40 and a nucleic acid encoding a second DMD-targeting gRNA targeting intron 55; or
ii) a nucleic acid encoding a first DMD-targeting gRNA that specifically hybridizes to a target nucleotide sequence in intron 40 and a nucleic acid encoding a second DMD-targeting gRNA that specifically hybridizes to a target nucleotide sequence in intron 55;
b) a nucleic acid comprising a donor DNA sequence encoding knock-in donor sequence of exons 41-55 of the DMD gene flanked on each side of the donor sequences by a genomic Cas9 cut site; and
c) a nucleic acid encoding a Cas9 enzyme or a functional fragment thereof, wherein binding of the complex to the target nucleic acid sequence results in increased DMD gene expression.

65. The gene editing complex of claim 64, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

66. The gene editing complex of claim 64 or 65, wherein the nucleic acid encoding a first DMD-targeting gRNA targeting intron 40 comprises the nucleotide sequence set forth in any one of SEQ ID NOs: 1-6 or a variant thereof comprising at least or about 80% identity to the nucleotide sequence set forth in any one of SEQ ID NOs: 1-6.

67. The gene editing complex of claim 64 or 65, wherein the nucleic acid encoding a first DMD-targeting gRNA that specifically hybridizes to a target nucleotide sequence in intron 40 comprising the nucleotide sequence set forth in any one of SEQ ID NOs: 112-117 or a variant thereof comprising at least or about 80% identity to the nucleotide sequence set forth in any one of SEQ ID NOs: 112-117.

68. The gene editing complex of any one of claims 64-67, wherein the nucleic acid encoding a first DMD-targeting gRNA targeting intron 55 comprises the nucleotide sequence set forth in any one of SEQ ID NOs: 7-9 or a variant thereof comprising at least or about 80% identity to the nucleotide sequence set forth in any one of SEQ ID NOs: 7-9.

69. The gene editing complex of any one of claims 64-67, wherein the nucleic acid encoding a first DMD-targeting gRNA that specifically hybridizes to a target nucleotide sequence in intron 55 comprising the nucleotide sequence set forth in any one of SEQ ID NOs: 118-120 or a variant thereof comprising at least or about 80% identity to the nucleotide sequence set forth in any one of SEQ ID NOs: 118-120.

70. The gene editing complex of any one of claims 64-69, wherein the nucleic acid encoding the knock-in donor sequence of exons 41-55 comprises the nucleotide sequence set forth in SEQ ID NO: 149, 152, 187, or 188 or a variant thereof comprising at least or about 80% identity to the nucleotide sequence set forth in SEQ ID NO: 149, 152, 187, or 188.

71. The gene editing complex of any one of claims 64-70, wherein the nucleic acid encoding the gRNA or the nucleic acid encoding the Cas9 enzyme further comprises a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

72. The gene editing complex of any one of claims 64-71, wherein the one or more nucleic acids are in a vector.

73. The gene editing complex of claim 72, wherein the vector is AAV.

74. A method of increasing expression of the DMD gene or increasing the expression of a functional dystrophin in a cell, wherein the method comprises contacting the cell with a nucleic acid comprising:
a) i) a nucleic acid encoding a DMD-targeting guide RNA (gRNA) targeting intron 19; or
ii) a nucleic acid encoding a DMD-targeting gRNA that specifically hybridizes to a target nucleotide sequence in intron 19;
b) a nucleic acid comprising a donor DNA sequence encoding knock-in donor sequence of exons 1-19 the DMD gene flanked on each side of the donor sequences by a genomic Cas9 cut site; and
c) a nucleic acid encoding a Cas9 enzyme or a functional fragment thereof.

75. A method of treating a subject suffering from one or more missing, duplicated, aberrant, or aberrantly-spliced exons or missing or aberrant introns in the DMD gene in a cell, the method comprising administering to the subject an effective amount of:
a) i) a nucleic acid encoding a DMD-targeting guide RNA (gRNA) targeting intron 19; or
ii) a nucleic acid encoding a DMD-targeting gRNA that specifically hybridizes to a target nucleotide sequence in intron 19;
b) a nucleic acid comprising a donor DNA sequence encoding knock-in donor sequence of exons 1-19 the DMD gene flanked on each side of the donor sequences by a genomic Cas9 cut site; and
c) a nucleic acid encoding a Cas9 enzyme or a functional fragment thereof.

76. The method of claim 74 or 75, wherein the Cas9 enzyme is encoded by the nucleotide sequence set out in SEQ ID NO: 161, 162, 181, or 183, a variant thereof comprising at least about 80% identity to the sequence set out in SEQ ID NO: 161, 162, 181, or 183, or a functional fragment thereof.

77. The method of any one of claims 74-76, wherein the nucleic acid encoding the DMD-targeting gRNA targeting intron 19 comprises the nucleotide sequence set forth in any one of SEQ ID NOs: 29-37 or a variant thereof comprising at least or about 80% identity to the nucleotide sequence set forth in any one of SEQ ID NOs: 29-37.

78. The method of any one of claims 74-76, wherein the nucleic acid encoding the DMD-targeting gRNA comprises a nucleotide sequence that specifically hybridizes to the target sequence in intron 19 comprising the nucleotide sequence set forth in any one of SEQ ID NOs: 140-148 or a variant thereof comprising at least or about 80% identity to the nucleotide sequence set forth in any one of SEQ ID NOs: 140-148.

79. The method of any one of claims 74-78, wherein the nucleic acid encoding the knock-in donor sequence of exons 1-19 comprises a nucleotide sequence selected from the group consisting of:
(a) the nucleotide sequence set forth in SEQ ID NO: 173 or 178 or a variant thereof comprising at least or about 80% identity to the nucleotide sequence set forth in SEQ ID NO: 173 or 178;
(b) the nucleotide sequence set forth in SEQ ID NO: 174 or a variant thereof comprising at least or about 80% identity to the nucleotide sequence set forth in SEQ ID NO: 174;
(c) the nucleotide sequence set forth in SEQ ID NO: 175 or a variant thereof comprising at least or about 80% identity to the nucleotide sequence set forth in SEQ ID NO: 175;
(d) the nucleotide sequence set forth in SEQ ID NO: 176 or a variant thereof comprising at least or about 80% identity to the nucleotide sequence set forth in SEQ ID NO: 176; and
(e) the nucleotide sequence set forth in SEQ ID NO: 177 or a variant thereof comprising at least or about 80% identity to the nucleotide sequence set forth in SEQ ID NO: 177.

80. The method of any one of claims 74-79, wherein the nucleic acid encoding the knock-in donor sequence of exons 1-19 comprises the nucleotide sequence set forth in SEQ ID NO: 172 or a variant thereof comprising at least or about 80% identity to the nucleotide sequence set forth in SEQ ID NO: 172.

81. The method of any one of claims 74-80, wherein expression of the nucleic acid encoding the gRNA or expression of the nucleic acid encoding the Cas9 enzyme is under the control of a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter.

82. The method of any one of claims 74-81, wherein the nucleic acid is in a vector.

83. The method of claim 82, wherein the vector is AAV.

84. The method of any one of claims 74-83, wherein the subject is a human subject.

85. The method of claim 84, wherein the human subject suffers from a muscular dystrophy.

86. The method of claim 85, wherein the muscular dystrophy is Duchene Muscular Dystrophy (DMD) or Becker Muscular Dystrophy (BMD).

87. A nucleic acid encoding a Cas enzyme comprising at its 5′ end a polynucleotide encoding a nuclear localization signal comprising a nucleotide sequence comprising:
(a) a nucleotide sequence comprising at least or about 70% identity to the nucleotide sequence set out in SEQ ID NO: 179; or
(b) a nucleotide sequence comprising at least or about 70% identity to a nucleotide sequence encoding the amino acid sequence set out in SEQ ID NO: 180.

88. A nucleic acid encoding a CRISPR-associated (Cas) enzyme comprising at its 5′ end a polynucleotide encoding a nuclear localization signal comprising a nucleotide sequence comprising:
(a) a nucleotide sequence comprising the nucleotide sequence set out in SEQ ID NO: 179 or a variant thereof comprising at least or about 70% identity to the nucleotide sequence set out in SEQ ID NO: 179; or
(b) a nucleotide sequence encoding the amino acid sequence set out in SEQ ID NO: 180 or a variant thereof comprising at least or about 70% identity to amino acid sequence set out in SEQ ID NO: 180.

89. The nucleic acid of claim 88, wherein the Cas enzyme is Cas9 or Cas13.

90. The nucleic acid of claim 89, wherein the Cas enzyme is Cas9.


SEQUENCE LISTING (188 sequences)

SEQ ID NO: 1 - RNA, 104 nt, Staphylococcus aureus
guauucauucaacauuacgucaaguuuuaguacucuggaaacagaaucuacuaaaacaaggcaaaaugccguguuuaucucgucaacuuguuggcgagauuuuu

SEQ ID NO: 2 - RNA, 103 nt, Staphylococcus aureus
guguuauucaauugacguaaugguuuuaguacucuggaaacagaaucuacuaaaacaaggcaaaaugccguguuuaucucgucaacuuguuggcgagauuuuu

SEQ ID NO: 3 - RNA, 104 nt, Staphylococcus aureus
gcugaugaaaugaaugggcuaacguuuuaguacucuggaaacagaaucuacuaaaacaaggcaaaaugccguguuuaucucgucaacuuguuggcgagauuuuu

SEQ ID NO: 4 - DNA, 104 bp, Staphylococcus aureus
gugugugaagaugcucugaugaaguuuuaguacucuggaaacagaaucuacuaaaacaaggcaaaaugccguguuuaucucgucaacuuguuggcgagauuuuu

SEQ ID NO: 5 - RNA, 104 nt, Staphylococcus aureus
gacaauaugcaaauaaaucuauaguuuuaguacucuggaaacagaaucuacuaaaacaaggcaaaaugccguguuuaucucgucaacuuguuggcgagauuuuu

SEQ ID NO: 6 - RNA, 104 nt, Staphylococcus aureus
guguggacggucccuaauaaauaguuuuaguacucuggaaacagaaucuacuaaaacaaggcaaaaugccguguuuaucucgucaacuuguuggcgagauuuuu

SEQ ID NO: 7 - RNA, 104 nt, Staphylococcus aureus
guuucuaagacgaggguguuaagguuuuaguacucuggaaacagaaucuacuaaaacaaggcaaaaugccguguuuaucucgucaacuuguuggcgagauuuuu

SEQ ID NO: 8 - RNA, 104 nt, Staphylococcus aureus
gacuuugcucagagaaauaacuuguuuuaguacucuggaaacagaaucuacuaaaacaaggcaaaaugccguguuuaucucgucaacuuguuggcgagauuuuu

SEQ ID NO: 9 - DNA, 104 bp, Staphylococcus aureus
gugugaaaauaagaaugagauggguuuuaguacucuggaaacagaaucuacuaaaacaaggcaaaaugccguguuuaucucgucaacuuguuggcgagauuuuu

SEQ ID NO: 10 - RNA, 105 nt, Staphylococcus aureus
gaaaaaucauccuuuagagaauaguuauaguacucuggaaacagaaucuacuauaacaaggcaaaaugccguguuuaucucgucaacuuguuggcgagauuuuuu

SEQ ID NO: 11 - RNA, 105 nt, Staphylococcus aureus
guaauaugaaaaaucauccuuuaguuauaguacucuggaaacagaaucuacuauaacaaggcaaaaugccguguuuaucucgucaacuuguuggcgagauuuuuu

SEQ ID NO: 12 - RNA, 105 nt, Staphylococcus aureus
guuagaacggaauguccauuccaguuauaguacucuggaaacagaaucuacuauaacaaggcaaaaugccguguuuaucucgucaacuuguuggcgagauuuuuu

SEQ ID NO: 13 - RNA, 105 nt, Staphylococcus aureus
gcuaggaucuaguuuucguaaauguuauaguacucuggaaacagaaucuacuauaacaaggcaaaaugccguguuuaucucgucaacuuguuggcgagauuuuuu

SEQ ID NO: 14 - RNA, 104 nt, Staphylococcus aureus
gcuuuaagcuuuucucaaugaaguuauaguacucuggaaacagaaucuacuauaacaaggcaaaaugccguguuuaucucgucaacuuguuggcgagauuuuuu

SEQ ID NO: 15 - RNA, 105 nt, Staphylococcus aureus
gcuuuucucaaugaacugaguaaguuauaguacucuggaaacagaaucuacuauaacaaggcaaaaugccguguuuaucucgucaacuuguuggcgagauuuuuu

SEQ ID NO: 16 - RNA, 105 nt, Staphylococcus aureus
guacuuuucucuuacacauucuaguuauaguacucuggaaacagaaucuacuauaacaaggcaaaaugccguguuuaucucgucaacuuguuggcgagauuuuuu

SEQ ID NO: 17 - RNA, 103 nt, Campylobacter jejuni
gaaaaucaucucuaauuugaucaguuauagucccugaaaagggacuauaauaaagaguuugcgggacucugcgggguuacaauccccuaaaaccgcuuuuuuu

SEQ ID NO: 18 - RNA, 103 nt, Campylobacter jejuni
guaauaugaaaaaucauccuuuaguuauagucccugaaaagggacuauaauaaagaguuugcgggacucugcgggguuacaauccccuaaaaccgcuuuuuuu

SEQ ID NO: 19 - RNA, 103 nt, Campylobacter jejuni
guaccccauagaauguguaagagguuauagucccugaaaagggacuauaauaaagaguuugcgggacucugcgggguuacaauccccuaaaaccgcuuuuuuu

SEQ ID NO: 20 - RNA, 103 nt, Campylobacter jejuni
gcucauuccuggcacucaucuuuguuauagucccugaaaagggacuauaauaaagaguuugcgggacucugcgggguuacaauccccuaaaaccgcuuuuuuu

SEQ ID NO: 21 - RNA, 103 nt, Campylobacter jejuni
gaucucuaauuugaucaauauguguuauagucccugaaaagggacuauaauaaagaguuugcgggacucugcgggguuacaauccccuaaaaccgcuuuuuuu

SEQ ID NO: 22 - RNA, 102 nt, Campylobacter jejuni
gauuucccuguugguacuuuucguuauagucccugaaaagggacuauaauaaagaguuugcgggacucugcgggguuacaauccccuaaaaccgcuuuuuuu

SEQ ID NO: 23 - RNA, 103 nt, Campylobacter jejuni
gucagcuucacagacagcauuuuguuauagucccugaaaagggacuauaauaaagaguuugcgggacucugcgggguuacaauccccuaaaaccgcuuuuuuu

SEQ ID NO: 24 - RNA, 103 nt, Campylobacter jejuni
gaaaccuggagguagagucauagguuauagucccugaaaagggacuauaauaaagaguuugcgggacucugcgggguuacaauccccuaaaaccgcuuuuuuu

SEQ ID NO: 25 - DNA, 103 bp, Campylobacter jejuni
guuugacaaaguucuaauuaacuguuauagucccugaaaagggacuauaauaaagaguuugcgggacucugcgggguuacaauccccuaaaaccgcuuuuuuu

SEQ ID NO: 26 - RNA, 103 nt, Campylobacter jejuni
gcauuucaaauucugcggaggcuguuauagucccugaaaagggacuauaauaaagaguuugcgggacucugcgggguuacaauccccuaaaaccgcuuuuuuu

SEQ ID NO: 27 - RNA, 102 nt, Campylobacter jejuni
guuuuacacugaaggcucauuuguuauagucccugaaaagggacuauaauaaagaguuugcgggacucugcgggguuacaauccccuaaaaccgcuuuuuuu

SEQ ID NO: 28 - RNA, 102 nt, Campylobacter jejuni
gaguacaggaaaaagcugaauaguuauagucccugaaaagggacuauaauaaagaguuugcgggacucugcgggguuacaauccccuaaaaccgcuuuuuuu

SEQ ID NO: 29 - DNA, 105 bp, Staphylococcus aureus
guucaaguaaugauccauuuccuguuauaguacucuggaaacagaaucuacuauaacaaggcaaaaugccguguuuaucucgucaacuuguuggcgagauuuuuu

SEQ ID NO: 30 - RNA, 105 nt, Staphylococcus aureus
gacagacuauuucagggguuguuguuauaguacucuggaaacagaaucuacuauaacaaggcaaaaugccguguuuaucucgucaacuuguuggcgagauuuuuu

SEQ ID NO: 31 - RNA, 105 nt, Staphylococcus aureus
guuguuuagaauaugagaugugaguuauaguacucuggaaacagaaucuacuauaacaaggcaaaaugccguguuuaucucgucaacuuguuggcgagauuuuuu

SEQ ID NO: 32 - RNA, 105 nt, Staphylococcus aureus
gcuguacacaaguaauaaaauuaguuauaguacucuggaaacagaaucuacuauaacaaggcaaaaugccguguuuaucucgucaacuuguuggcgagauuuuuu

SEQ ID NO: 33 - RNA, 104 nt, Staphylococcus aureus
gggguuguuuagaauaugagauguuauaguacucuggaaacagaaucuacuauaacaaggcaaaaugccguguuuaucucgucaacuuguuggcgagauuuuuu

SEQ ID NO: 34 - RNA, 105 nt, Staphylococcus aureus
gcacauagguucauauuuaucagguuauaguacucuggaaacagaaucuacuauaacaaggcaaaaugccguguuuaucucgucaacuuguuggcgagauuuuuu

SEQ ID NO: 35 - DNA, 105 bp, Staphylococcus aureus
guaaacaaccccugaaauagucuguuauaguacucuggaaacagaaucuacuauaacaaggcaaaaugccguguuuaucucgucaacuuguuggcgagauuuuuu

SEQ ID NO: 36 - RNA, 102 nt, Campylobacter jejuni
guacacaaguaauaaaauuaauguuauagucccugaaaagggacuauaauaaagaguuugcgggacucugcgggguuacaauccccuaaaaccgcuuuuuuu

SEQ ID NO: 37 - RNA, 103 nt, Campylobacter jejuni
gugaaauagucugugaauaaauaguuauagucccugaaaagggacuauaauaaagaguuugcgggacucugcgggguuacaauccccuaaaaccgcuuuuuuu

SEQ ID NO: 38 - RNA, 23 nt, Staphylococcus aureus
guauucauucaacauuacgucaa

SEQ ID NO: 39 - RNA, 22 nt, Staphylococcus aureus
guguuauucaauugacguaaug

SEQ ID NO: 40 - RNA, 23 nt, Staphylococcus aureus
gcugaugaaaugaaugggcuaac

SEQ ID NO: 41 - RNA, 23 nt, Staphylococcus aureus
gugugugaagaugcucugaugaa

SEQ ID NO: 42 - RNA, 23 nt, Staphylococcus aureus
gacaauaugcaaauaaaucuaua

SEQ ID NO: 43 - RNA, 23 nt, Staphylococcus aureus
guguggacggucccuaauaaaua

SEQ ID NO: 44 - RNA, 23 nt, Staphylococcus aureus
guuucuaagacgaggguguuaag

SEQ ID NO: 45 - RNA, 23 nt, Staphylococcus aureus
gacuuugcucagagaaauaacuu

SEQ ID NO: 46 - RNA, 23 nt, Staphylococcus aureus
gugugaaaauaagaaugagaugg

SEQ ID NO: 47 - RNA, 23 nt, Staphylococcus aureus
gaaaaaucauccuuuagagaaua

SEQ ID NO: 48 - RNA, 23 nt, Staphylococcus aureus
guaauaugaaaaaucauccuuua

SEQ ID NO: 49 - RNA, 23 nt, Staphylococcus aureus
guuagaacggaauguccauucca

SEQ ID NO: 50 - RNA, 23 nt, Staphylococcus aureus
gcuaggaucuaguuuucguaaau

SEQ ID NO: 51 - RNA, 22 nt, Staphylococcus aureus
gcuuuaagcuuuucucaaugaa

SEQ ID NO: 52 - DNA, 23 bp, Staphylococcus aureus
gcuuuucucaaugaacugaguaa

SEQ ID NO: 53 - RNA, 23 nt, Staphylococcus aureus
guacuuuucucuuacacauucua

SEQ ID NO: 54 - RNA, 23 nt, Campylobacter jejuni
gaaaaucaucucuaauuugauca

SEQ ID NO: 55 - RNA, 23 nt, Campylobacter jejuni
guaauaugaaaaaucauccuuua

SEQ ID NO: 56 - RNA, 23 nt, Campylobacter jejuni
guaccccauagaauguguaagag

SEQ ID NO: 57 - DNA, 23 bp, Campylobacter jejuni
gcucauuccuggcacucaucuuu

SEQ ID NO: 58 - RNA, 23 nt, Campylobacter jejuni
gaucucuaauuugaucaauaugu

SEQ ID NO: 59 - RNA, 22 nt, Campylobacter jejuni
gauuucccuguugguacuuuuc

SEQ ID NO: 60 - RNA, 23 nt, Campylobacter jejuni
gucagcuucacagacagcauuuu

SEQ ID NO: 61 - RNA, 23 nt, Campylobacter jejuni
gaaaccuggagguagagucauag

SEQ ID NO: 62 - RNA, 23 nt, Campylobacter jejuni
guuugacaaaguucuaauuaacu

SEQ ID NO: 63 - RNA, 23 nt, Campylobacter jejuni
gcauuucaaauucugcggaggcu

SEQ ID NO: 64 - RNA, 22 nt, Campylobacter jejuni
guuuuacacugaaggcucauuu

SEQ ID NO: 65 - RNA, 22 nt, Campylobacter jejuni
gaguacaggaaaaagcugaaua

SEQ ID NO: 66 - RNA, 23 nt, Staphylococcus aureus
guucaaguaaugauccauuuccu

SEQ ID NO: 67 - RNA, 23 nt, Staphylococcus aureus
gacagacuauuucagggguuguu

SEQ ID NO: 68 - RNA, 23 nt, Staphylococcus aureus
guuguuuagaauaugagauguga

SEQ ID NO: 69 - RNA, 23 nt, Staphylococcus aureus
gcuguacacaaguaauaaaauua

SEQ ID NO: 70 - DNA, 22 bp, Staphylococcus aureus
gggguuguuuagaauaugagau

SEQ ID NO: 71 - RNA, 23 nt, Staphylococcus aureus
gcacauagguucauauuuaucag

SEQ ID NO: 72 - RNA, 23 nt, Staphylococcus aureus
guaaacaaccccugaaauagucu

SEQ ID NO: 73 - RNA, 22 nt, Campylobacter jejuni
guacacaaguaauaaaauuaau

SEQ ID NO: 74 - RNA, 23 nt, Campylobacter jejuni
gugaaauagucugugaauaaaua

SEQ ID NO: 75 - RNA, 81 nt, Staphylococcus aureus
guuuuaguacucuggaaacagaaucuacuaaaacaaggcaaaaugccguguuuaucucgucaacuuguuggcgagauuuuu

SEQ ID NO: 76 - RNA, 81 nt, Staphylococcus aureus; identical to SEQ ID NO: 75

SEQ ID NO: 77 - RNA, 81 nt, Staphylococcus aureus; identical to SEQ ID NO: 75

SEQ ID NO: 78 - RNA, 81 nt, Staphylococcus aureus; identical to SEQ ID NO: 75

SEQ ID NO: 79 - RNA, 81 nt, Staphylococcus aureus; identical to SEQ ID NO: 75

SEQ ID NO: 80 - RNA, 81 nt, Staphylococcus aureus; identical to SEQ ID NO: 75

SEQ ID NO: 81 - RNA, 81 nt, Staphylococcus aureus; identical to SEQ ID NO: 75

SEQ ID NO: 82 - RNA, 81 nt, Staphylococcus aureus; identical to SEQ ID NO: 75

SEQ ID NO: 83 - RNA, 81 nt, Staphylococcus aureus; identical to SEQ ID NO: 75

SEQ ID NO: 84 - DNA, 82 bp, Staphylococcus aureus
guuauaguacucuggaaacagaaucuacuauaacaaggcaaaaugccguguuuaucucgucaacuuguuggcgagauuuuuu

SEQ ID NO: 85 - RNA, 82 nt, Staphylococcus aureus
guuauaguacucuggaaacagaaucuacuauaacaaggcaaaaugccguguuuaucucgucaacuuguuggcgagauuuuuu

SEQ ID NO: 86 - RNA, 82 nt, Staphylococcus aureus; identical to SEQ ID NO: 85

SEQ ID NO: 87 - RNA, 82 nt, Staphylococcus aureus; identical to SEQ ID NO: 85

SEQ ID NO: 88 - RNA, 82 nt, Staphylococcus aureus; identical to SEQ ID NO: 85

SEQ ID NO: 89 - RNA, 82 nt, Staphylococcus aureus; identical to SEQ ID NO: 85

SEQ ID NO: 90 - RNA, 82 nt, Staphylococcus aureus; identical to SEQ ID NO: 85

SEQ ID NO: 91 - DNA, 80 bp, Campylobacter jejuni
guuauagucccugaaaagggacuauaauaaagaguuugcgggacucugcgggguuacaauccccuaaaaccgcuuuuuuu

SEQ ID NO: 92 - RNA, 80 nt, Campylobacter jejuni
guuauagucccugaaaagggacuauaauaaagaguuugcgggacucugcgggguuacaauccccuaaaaccgcuuuuuuu

SEQ ID NO: 93 - RNA, 80 nt, Campylobacter jejuni; identical to SEQ ID NO: 92

SEQ ID NO: 94 - RNA, 80 nt, Campylobacter jejuni; identical to SEQ ID NO: 92

SEQ ID NO: 95 - RNA, 80 nt, Campylobacter jejuni; identical to SEQ ID NO: 92

SEQ ID NO: 96 - RNA, 80 nt, Campylobacter jejuni; identical to SEQ ID NO: 92

SEQ ID NO: 97 - RNA, 80 nt, Campylobacter jejuni; identical to SEQ ID NO: 92

SEQ ID NO: 98 - RNA, 80 nt, Campylobacter jejuni; identical to SEQ ID NO: 92

SEQ ID NO: 99 - RNA, 80 nt, Campylobacter jejuni; identical to SEQ ID NO: 92

SEQ ID NO: 100 - RNA, 80 nt, Campylobacter jejuni; identical to SEQ ID NO: 92

SEQ ID NO: 101 - RNA, 80 nt, Campylobacter jejuni; identical to SEQ ID NO: 92

SEQ ID NO: 102 - RNA, 80 nt, Campylobacter jejuni; identical to SEQ ID NO: 92

SEQ ID NO: 103 - RNA, 82 nt, Staphylococcus aureus; identical to SEQ ID NO: 85

SEQ ID NO: 104 - RNA, 82 nt, Staphylococcus aureus; identical to SEQ ID NO: 85

SEQ ID NO: 105 - DNA, 82 bp, Staphylococcus aureus; identical to SEQ ID NO: 84

SEQ ID NO: 106 - RNA, 82 nt, Staphylococcus aureus; identical to SEQ ID NO: 85

SEQ ID NO: 107 - RNA, 82 nt, Staphylococcus aureus; identical to SEQ ID NO: 85

SEQ ID NO: 108 - RNA, 82 nt, Staphylococcus aureus; identical to SEQ ID NO: 85

SEQ ID NO: 109 - RNA, 82 nt, Staphylococcus aureus; identical to SEQ ID NO: 85

SEQ ID NO: 110 - RNA, 80 nt, Campylobacter jejuni; identical to SEQ ID NO: 92

SEQ ID NO: 111 - RNA, 80 nt, Campylobacter jejuni; identical to SEQ ID NO: 92

SEQ ID NO: 112 - DNA, 28 bp, Staphylococcus aureus
attcaattgacgtaatgttgaatgaata

SEQ ID NO: 113 - DNA, 28 bp, Staphylococcus aureus
gtgttattcaattgacgtaatgttgaat

SEQ ID NO: 114 - DNA, 28 bp, Staphylococcus aureus
actcatgttagcccattcatttcatcag

SEQ ID NO: 115 - DNA, 28 bp, Staphylococcus aureus
attcatttcatcagagcatcttcacaca

SEQ ID NO: 116 - DNA, 28 bp, Staphylococcus aureus
actctctatagatttatttgcatattgt

SEQ ID NO: 117 - DNA, 28 bp, Staphylococcus aureus
tgtggacggtccctaataaataatgagt

SEQ ID NO: 118 - DNA, 28 bp, Staphylococcus aureus
attccacttaacaccctcgtcttagaaa

SEQ ID NO: 119 - DNA, 28 bp, Staphylococcus aureus
actttgctcagagaaataacttagggat

SEQ ID NO: 120 - DNA, 28 bp, Staphylococcus aureus
tgtgaaaataagaatgagatggctgaat

SEQ ID NO: 121 - DNA, 28 bp, Staphylococcus aureus
aaaaatcatcctttagagaatacagaat

SEQ ID NO: 122 - DNA, 28 bp, Staphylococcus aureus
taatatgaaaaatcatcctttagagaat

SEQ ID NO: 123 - DNA, 28 bp, Staphylococcus aureus
ttagaacggaatgtccattccagagagt

SEQ ID NO: 124 - DNA, 28 bp, Staphylococcus aureus
ctaggatctagttttcgtaaattagagt

SEQ ID NO: 125 - DNA, 28 bp, Staphylococcus aureus
actcagttcattgagaaaagcttaaagc

SEQ ID NO: 126 - DNA, 28 bp, Staphylococcus aureus
attccattactcagttcattgagaaaag

SEQ ID NO: 127 - DNA, 28 bp, Staphylococcus aureus
accccatagaatgtgtaagagaaaagta

SEQ ID NO: 128 - DNA, 30 bp, Campylobacter jejuni
aaaatcatctctaatttgatcaatatgtac

SEQ ID NO: 129 - DNA, 30 bp, Campylobacter jejuni
taatatgaaaaatcatcctttagagaatac

SEQ ID NO: 130 - DNA, 30 bp, Campylobacter jejuni
taccccatagaatgtgtaagagaaaagtac

SEQ ID NO: 131 - DNA, 30 bp, Campylobacter jejuni
ctcattcctggcactcatctttatttgcac

SEQ ID NO: 132 - DNA, 30 bp, Campylobacter jejuni
atctctaatttgatcaatatgtacttacac

SEQ ID NO: 133 - DNA, 30 bp, Campylobacter jejuni
gtgtaagagaaaagtaccaacagggaaatc

SEQ ID NO: 134 - DNA, 30 bp, Campylobacter jejuni
gtgtttgcaaaatgctgtctgtgaagctga

SEQ ID NO: 135 - DNA, 30 bp, Campylobacter jejuni
gtgcttggctatgactctacctccaggttt

SEQ ID NO: 136 - DNA, 30 bp, Campylobacter jejuni
gtacaaccagttaattagaactttgtcaaa

SEQ ID NO: 137 - DNA, 30 bp, Campylobacter jejuni
gtgtagccagcctccgcagaatttgaaatg

SEQ ID NO: 138 - DNA, 30 bp, Campylobacter jejuni
gtataatgaaatgagccttcagtgtaaaac

SEQ ID NO: 139 - DNA, 30 bp, Campylobacter jejuni
gtataacgtattcagctttttcctgtactc

SEQ ID NO: 140 - DNA, 28 bp, Staphylococcus aureus
ttcaagtaatgatccatttcctctgggt

SEQ ID NO: 141 - DNA, 28 bp, Staphylococcus aureus
acagactatttcaggggttgtttagaat

SEQ ID NO: 142 - DNA, 28 bp, Staphylococcus aureus
ttgtttagaatatgagatgtgaatggat

SEQ ID NO: 143 - DNA, 28 bp, Staphylococcus aureus
ctgtacacaagtaataaaattaatggat

SEQ ID NO: 144 - DNA, 28 bp, Staphylococcus aureus
ggggttgtttagaatatgagatgtgaat

SEQ ID NO: 145 - DNA, 28 bp, Staphylococcus aureus
cacataggttcatatttatcagctgaat

SEQ ID NO: 146 - DNA, 28 bp, Staphylococcus aureus
attcacagactatttcaggggttgttta

SEQ ID NO: 147 - DNA, 30 bp, Staphylococcus aureus
gtacacaagtaataaaattaatggatacac

SEQ ID NO: 148 - DNA, 30 bp, Staphylococcus aureus
gtgcaatatatttattcacagactatttca

SEQ ID NO: 149 - DNA, 2802 bp, Artificial Seuqence
  Synthetic Polynucleotide
attcagccatctcattcttattttcacagcgaggaagcggaagagcgccgcggccgcacaacagcctttgaaattttgagagaagtatttgctgcttgcaagtcggttgatgtggttagctaactgccctgggccctgtattggttttgctcaataggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagcactcttgtggatccaattgaacaattctcagcatttgtacttgtaactgacaagccagggacaaaacaaaatagtgcggccgcggcgcgccgacgaaagggcctcgtgatacgcactcattatttattagggaccgtccaca

SEQ ID NO: 150 - DNA, 28 bp, Artificial Seuqence
  Synthetic Polynucleotide
attcagccatctcattcttattttcaca

SEQ ID NO: 151 - DNA, 100 bp, Artificial Seuqence
  Synthetic Polynucleotide
acaacagcctttgaaattttgagagaagtatttgctgcttgcaagtcggttgatgtggttagctaactgccctgggccctgtattggttttgctcaatag

SEQ ID NO: 152 - DNA, 2478 bp, Artificial Seuqence
  Synthetic Polynucleotide
gaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaa

SEQ ID NO: 153 - DNA, 100 bp, Artificial Seuqence
  Synthetic Polynucleotide
gtaagtcaggcatttccgctttagcactcttgtggatccaattgaacaattctcagcatttgtacttgtaactgacaagccagggacaaaacaaaatagt

SEQ ID NO: 154 - DNA, 28 bp, Artificial Seuqence
  Synthetic Polynucleotide
actcattatttattagggaccgtccaca

SEQ ID NO: 155 - DNA, 2538 bp, Artificial Seuqence
  Synthetic Polynucleotide
atccattaattttattacttgtgtacagggccaatagaaactgggcttgtcgagacagagaagattcttgcgtttctgataggcacctattggtcttactgacatccactttgcctttctctccacagatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatggtaagtatcaaggttacaagacaggtttaaggaactctctggaatggacattccgttctaa

SEQ ID NO: 156 - DNA, 28 bp, Artificial Seuqence
  Synthetic Polynucleotide
atccattaattttattacttgtgtacag

SEQ ID NO: 157 - DNA, 100 bp, Artificial Seuqence
  Synthetic Polynucleotide
ggccaatagaaactgggcttgtcgagacagagaagattcttgcgtttctgataggcacctattggtcttactgacatccactttgcctttctctccacag

SEQ ID NO: 158 - DNA, 2349 bp, Artificial Seuqence
  Synthetic Polynucleotide
atgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatg

SEQ ID NO: 159 - DNA, 33 bp, Artificial Seuqence
  Synthetic Polynucleotide
gtaagtatcaaggttacaagacaggtttaagga

SEQ ID NO: 160 - DNA, 28 bp, Artificial Seuqence
  Synthetic Polynucleotide
actctctggaatggacattccgttctaa

SEQ ID NO: 161 - DNA, 3258 bp, Staphylococcus aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 162 - DNA, 3030 bp, Campylobacter jejuni
atggccccaaagaagaagcggaaggtcggtgctcgcatactcgcttttgatattggaatttcatccataggatgggcattttcagaaaatgatgaacttaaagattgtggagtcaggattttcacaaaagtagagaatcccaaaacaggggaaagccttgctctcccaaggagactggcgcgatccgcaaggaaacgacttgctaggcgcaaagcaaggttgaatcatcttaaacatctcattgctaatgaatttaaactcaattatgaagattaccaaagttttgatgaatctttggctaaagcgtataaaggtagtctcatttccccatatgaactccgttttcgcgcattgaatgaacttctctctaaacaagattttgctcgtgtcattcttcacattgcaaaacgtcgcggttatgatgatattaagaattcagatgataaggaaaagggagcgattctcaaagctattaaacaaaatgaggagaaattggctaactatcaatctgtcggagaatatctctataaggaatatttccaaaagtttaaggaaaattccaaggaatttacaaatgtgcgaaataagaaggagtcctatgaaaggtgcattgctcaatcctttctcaaagacgaactcaaactcatctttaagaaacaaagggaatttgggtttagttttagtaagaagtttgaagaggaagtattgtcagtggctttctataaacgggctctcaaggacttttctcatctggtcggaaattgttctttctttacggatgaaaagcgggcaccgaagaattcaccactcgcgtttatgtttgtcgcactcactcgcattattaatctcctcaataaccttaagaatacagaaggaattctttatacaaaagatgatctcaatgcgctgcttaatgaagttttgaagaatggaactcttacttataaacaaacaaagaagttgcttgggttgtcagatgattatgaattcaaaggagagaaaggtacttattttatcgagtttaagaaatataaagagtttattaaagcactcggagaacataatctctcccaagacgaccttaatgaaattgcaaaagatattacactcattaaagatgaaataaaactgaagaaagcacttgcaaaatatgatctgaatcaaaatcaaatcgattcactttctaaattggagtttaaagaccatttgaatatttctttcaaagcacttaaattggtcacaccactcatgcttgaggggaagaaatacgatgaagcctgtaatgagcttaatttgaaagtcgctattaatgaagataagaaggattttcttccagcttttaatgaaacctattataaagatgaggttacgaatccggttgtcttgcgagcaattaaggaatataggaaagtactcaacgctttgctcaagaagtatggtaaagtacataaaattaatattgaacttgcccgcgaggtcggtaagaatcattcacaacgggctaaaattgaaaaggagcaaaatgaaaattataaagcgaagaaagacgcagaactcgagtgtgaaaagttgggcctcaaaattaattccaagaatatactcaagcttcggctgtttaaggaacaaaaggagttttgtgcatatagtggagagaaaatcaaaatctccgatcttcaagacgaaaagatgctggaaattgaccatatttatccatattctaggtcttttgatgatagttatatgaataaagtccttgtatttacaaaacaaaaccaggagaaacttaaccaaactccctttgaggcttttgggaatgattccgcaaaatggcaaaagattgaagtattggctaagaatctcccgaccaagaaacagaaacgaattttggataagaactataaagataaagagcagaagaattttaaagatagaaatctcaatgatactcgatacattgctcgccttgtcttgaattataccaaagactatttggactttctccccctctcagatgatgaaaataccaaattgaatgacactcaaaagggatcaaaagtccatgttgaggccaaaagtgggatgctcacttccgcactccgccatacgtggggattttccgcaaaagacaggaataatcacctgcatcatgctatagatgctgttataatagcatatgcaaataattccattgtcaaagccttttctgattttaagaaggaacaggaaagtaattctgcagaattgtatgctaagaagatttccgaactcgattataagaataaaagaaaattctttgaaccatttagtgggtttcggcaaaaggtcttggacaaaattgatgaaatatttgtcagcaaaccagaaaggaagaaaccatccggagcgcttcatgaagagacttttcggaaggaagaggaattttatcaaagctatggcggaaaagagggagttcttaaagcgttggagctcggtaaaatacggaaggtcaatggtaaaatagttaagaacggggatatgtttagggttgatatatttaaacataagaaaacaaataaattttatgctgttcccatttatactatggactttgcattgaaagtcttgccgaataaagcggtcgctaggtccaagaaaggagagattaaagactggatattgatggatgaaaactacgaattttgcttttccttgtataaagatagcctgattttgatacaaaccaaagatatgcaggaaccagaatttgtttattataatgcgtttacaagtagtactgtcagccttattgtctccaaacatgacaataaatttgaaaccctcagtaagaatcagaaaattttgtttaagaatgcgaatgagaaagaggttattgcaaaatccattggaattcaaaatttgaaggtattcgagaagtatattgtcagcgcgctcggagaggttactaaagctgaattccgccaacgcgaagatttcaagaaaaaaaggccggcggccacgaaaaaggccggccaggcaaaaaagaaaaagtga

SEQ ID NO: 163 - protein, 6 aa, Staphylococcus aureus
NNGRRT

SEQ ID NO: 164 - protein, 8 aa, Staphylococcus aureus
NNNNRYAC

SEQ ID NO: 165 - RNA, 104 nt, Artificial Seuqence
  Synthetic Polynucleotide
gaucauacaguauuugaacgacuguuuuaguacucuggaaacagaaucuacuaaaacaaggcaaaaugccguguuuaucucgucaacuuguuggcgagauuuuu

SEQ ID NO: 166 - RNA, 104 nt, Artificial Seuqence
  Synthetic Polynucleotide
gcacccagcagaagaagauaugaguuuuaguacucuggaaacagaaucuacuaaaacaaggcaaaaugccguguuuaucucgucaacuuguuggcgagauuuuu

SEQ ID NO: 167 - RNA, 104 nt, Artificial Seuqence
  Synthetic Polynucleotide
gcuuagauugcuauucuaaaaagguuuuaguacucuggaaacagaaucuacuaaaacaaggcaaaaugccguguuuaucucgucaacuuguuggcgagauuuuu

SEQ ID NO: 168 - DNA, 28 bp, Artificial Seuqence
  Synthetic Polynucleotide
atcatacagtatttgaacgactatgggt

SEQ ID NO: 169 - RNA, 28 nt, Artificial Seuqence
  Synthetic Polynucleotide
cacccagcagaagaagauaugagggaau

SEQ ID NO: 170 - DNA, 28 bp, Artificial Seuqence
  Synthetic Polynucleotide
cttagattgctattctaaaaagtagagt

SEQ ID NO: 171 - DNA, 1794 bp, Artificial Seuqence
  Synthetic Polynucleotide
attccctcatatcttcttctgctgggtgcaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataagagatctggatccctcgaggctagcgcggccgcgtttaaacagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggacccatagtcgttcaaatactgtatgat

SEQ ID NO: 172 - DNA, 3525 bp, Artificial Sequence
  Synthetic
atccattaattttattacttgtgtacaggaattcaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcgggaattcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaagccaccatggccctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatggtaagtatcaaggttacaagacaggtttaaggaatccattaattttattacttgtgtacag

SEQ ID NO: 173 - DNA, 28 bp, Artificial Sequence
  Synthetic
atccattaattttattacttgtgtacag

SEQ ID NO: 174 - DNA, 797 bp, Artificial Sequence
  Synthetic
aaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcg

SEQ ID NO: 175 - DNA, 237 bp, Artificial Sequence
  Synthetic
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaa

SEQ ID NO: 176 - DNA, 2389 bp, Artificial Sequence
  Synthetic
gccaccatggccctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatg

SEQ ID NO: 177 - DNA, 33 bp, Artificial Sequence; identical to SEQ ID NO: 159
  Synthetic

SEQ ID NO: 178 - DNA, 28 bp, Artificial Sequence
  Synthetic
atccattaattttattacttgtgtacag

SEQ ID NO: 179 - DNA, 111 bp, Artificial Sequence
  Synthetic
atggccgatcccaggggtatcttgaaggcatttcccaagcggcagaaaattcatgctgatgcatcatcaaaagtacttgcaaagattcctaggagggaagagggagaagaa

SEQ ID NO: 180 - protein, 37 aa, Artificial Sequence
  Synthetic
MADPRGILKAFPKRQKIHADASSKVLAKIPRREEGEE

SEQ ID NO: 181 - DNA, 3318 bp, Staphylococcus aureus
atggccgatcccaggggtatcttgaaggcatttcccaagcggcagaaaattcatgctgatgcatcatcaaaagtacttgcaaagattcctaggagggaagagggagaagaaaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 182 - protein, 1105 aa, Staphylococcus aureus
MADPRGILKAFPKRQKIHADASSKVLAKIPRREEGEEKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 183 - DNA, 3111 bp, Campylobacter jejuni
atggccgatcccaggggtatcttgaaggcatttcccaagcggcagaaaattcatgctgatgcatcatcaaaagtacttgcaaagattcctaggagggaagagggagaagaagctcgcatactcgcttttgatattggaatttcatccataggatgggcattttcagaaaatgatgaacttaaagattgtggagtcaggattttcacaaaagtagagaatcccaaaacaggggaaagccttgctctcccaaggagactggcgcgatccgcaaggaaacgacttgctaggcgcaaagcaaggttgaatcatcttaaacatctcattgctaatgaatttaaactcaattatgaagattaccaaagttttgatgaatctttggctaaagcgtataaaggtagtctcatttccccatatgaactccgttttcgcgcattgaatgaacttctctctaaacaagattttgctcgtgtcattcttcacattgcaaaacgtcgcggttatgatgatattaagaattcagatgataaggaaaagggagcgattctcaaagctattaaacaaaatgaggagaaattggctaactatcaatctgtcggagaatatctctataaggaatatttccaaaagtttaaggaaaattccaaggaatttacaaatgtgcgaaataagaaggagtcctatgaaaggtgcattgctcaatcctttctcaaagacgaactcaaactcatctttaagaaacaaagggaatttgggtttagttttagtaagaagtttgaagaggaagtattgtcagtggctttctataaacgggctctcaaggacttttctcatctggtcggaaattgttctttctttacggatgaaaagcgggcaccgaagaattcaccactcgcgtttatgtttgtcgcactcactcgcattattaatctcctcaataaccttaagaatacagaaggaattctttatacaaaagatgatctcaatgcgctgcttaatgaagttttgaagaatggaactcttacttataaacaaacaaagaagttgcttgggttgtcagatgattatgaattcaaaggagagaaaggtacttattttatcgagtttaagaaatataaagagtttattaaagcactcggagaacataatctctcccaagacgaccttaatgaaattgcaaaagatattacactcattaaagatgaaataaaactgaagaaagcacttgcaaaatatgatctgaatcaaaatcaaatcgattcactttctaaattggagtttaaagaccatttgaatatttctttcaaagcacttaaattggtcacaccactcatgcttgaggggaagaaatacgatgaagcctgtaatgagcttaatttgaaagtcgctattaatgaagataagaaggattttcttccagcttttaatgaaacctattataaagatgaggttacgaatccggttgtcttgcgagcaattaaggaatataggaaagtactcaacgctttgctcaagaagtatggtaaagtacataaaattaatattgaacttgcccgcgaggtcggtaagaatcattcacaacgggctaaaattgaaaaggagcaaaatgaaaattataaagcgaagaaagacgcagaactcgagtgtgaaaagttgggcctcaaaattaattccaagaatatactcaagcttcggctgtttaaggaacaaaaggagttttgtgcatatagtggagagaaaatcaaaatctccgatcttcaagacgaaaagatgctggaaattgaccatatttatccatattctaggtcttttgatgatagttatatgaataaagtccttgtatttacaaaacaaaaccaggagaaacttaaccaaactccctttgaggcttttgggaatgattccgcaaaatggcaaaagattgaagtattggctaagaatctcccgaccaagaaacagaaacgaattttggataagaactataaagataaagagcagaagaattttaaagatagaaatctcaatgatactcgatacattgctcgccttgtcttgaattataccaaagactatttggactttctccccctctcagatgatgaaaataccaaattgaatgacactcaaaagggatcaaaagtccatgttgaggccaaaagtgggatgctcacttccgcactccgccatacgtggggattttccgcaaaagacaggaataatcacctgcatcatgctatagatgctgttataatagcatatgcaaataattccattgtcaaagccttttctgattttaagaaggaacaggaaagtaattctgcagaattgtatgctaagaagatttccgaactcgattataagaataaaagaaaattctttgaaccatttagtgggtttcggcaaaaggtcttggacaaaattgatgaaatatttgtcagcaaaccagaaaggaagaaaccatccggagcgcttcatgaagagacttttcggaaggaagaggaattttatcaaagctatggcggaaaagagggagttcttaaagcgttggagctcggtaaaatacggaaggtcaatggtaaaatagttaagaacggggatatgtttagggttgatatatttaaacataagaaaacaaataaattttatgctgttcccatttatactatggactttgcattgaaagtcttgccgaataaagcggtcgctaggtccaagaaaggagagattaaagactggatattgatggatgaaaactacgaattttgcttttccttgtataaagatagcctgattttgatacaaaccaaagatatgcaggaaccagaatttgtttattataatgcgtttacaagtagtactgtcagccttattgtctccaaacatgacaataaatttgaaaccctcagtaagaatcagaaaattttgtttaagaatgcgaatgagaaagaggttattgcaaaatccattggaattcaaaatttgaaggtattcgagaagtatattgtcagcgcgctcggagaggttactaaagctgaattccgccaacgcgaagatttcaagaaaaaaaggccggcggccacgaaaaaggccggccaggcaaaaaagaaaaagtga

SEQ ID NO: 184 - protein, 1036 aa, Campylobacter jejuni
MADPRGILKAFPKRQKIHADASSKVLAKIPRREEGEEARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKKRPAATKKAGQAKKKK

SEQ ID NO: 185 - DNA, 31 bp, Artificial Sequence
  synthetic
agcttgatttccaatgggaaaaagttaacaa

SEQ ID NO: 186 - DNA, 24 bp, Artificial Sequence
  Synthetic
atctgcttcctccaaccataaaac

SEQ ID NO: 187 - DNA, 3500 bp, Artificial Sequence
  Synthetic
attcagccatctcattcttattttcacatcttgcgtttctgataggcacctattggtcttactgacatccactttgcctttctctccacaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaaggtataaaatcttaccttttattcaaattataagttttgcgtatgtgtaaagccaaataacacaccaaaacacataaaagcaaagcatcgttgggttgtctaaagcattatgttacttcatccctgaccaatacagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtatggggcttttagaatttggggaggggtctcaactttatttcacttccctgtgcattctgaaaagcctcattcttaatgtctgattttcaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaaggtaaatgtaaccaagtataaccagatagccagtttctgaatcatgggagtggggagtaataaaatattttgcaaccttttactctttaataaactttaattttcacattcttctaattttatgctaaatgtcttttacagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggtgagttgctcaacaatgtaaaatttaccctatctgaatctgcagtttattagttcagtcatgctaacaaaactgtatcatttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctgtaagtcaagattagctaattatataggagaggggttgcttggttgtgtagggtgaaaaaaggcataaaatatcttgatgatttgtaggaataactatataaatgatgttctttctttccttctaaccctcactccaaacagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaagtatgctctacttgtcagccacgtttttgtattttctctgcaagacttcctgatacacccctgcattgatcaagggtcatcaatggaaacgtattctgacttcatccactgtccacttctttcagttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaaggtaaatagttttatcaaatagtccaccccaaaatcattttttttgcctttagttttatatttcttctttaaagtgcttcaattaataagttctttcttttttttcttgatagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtatcaaggttacaagacaggtttaaggaggccaatagaaactgggcttgtcgagacagagaagatactcattatttattagggaccgtccaca

SEQ ID NO: 188 - DNA, 3444 bp, Artificial Sequence
  Synthetic
tcttgcgtttctgataggcacctattggtcttactgacatccactttgcctttctctccacaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaaggtataaaatcttaccttttattcaaattataagttttgcgtatgtgtaaagccaaataacacaccaaaacacataaaagcaaagcatcgttgggttgtctaaagcattatgttacttcatccctgaccaatacagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtatggggcttttagaatttggggaggggtctcaactttatttcacttccctgtgcattctgaaaagcctcattcttaatgtctgattttcaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaaggtaaatgtaaccaagtataaccagatagccagtttctgaatcatgggagtggggagtaataaaatattttgcaaccttttactctttaataaactttaattttcacattcttctaattttatgctaaatgtcttttacagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggtgagttgctcaacaatgtaaaatttaccctatctgaatctgcagtttattagttcagtcatgctaacaaaactgtatcatttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctgtaagtcaagattagctaattatataggagaggggttgcttggttgtgtagggtgaaaaaaggcataaaatatcttgatgatttgtaggaataactatataaatgatgttctttctttccttctaaccctcactccaaacagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaagtatgctctacttgtcagccacgtttttgtattttctctgcaagacttcctgatacacccctgcattgatcaagggtcatcaatggaaacgtattctgacttcatccactgtccacttctttcagttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaaggtaaatagttttatcaaatagtccaccccaaaatcattttttttgcctttagttttatatttcttctttaaagtgcttcaattaataagttctttcttttttttcttgatagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtatcaaggttacaagacaggtttaaggaggccaatagaaactgggcttgtcgagacagagaagat